(19) United States
(12) Patent Application Publication
(10) Pub. No.: US 2004/0131582 A1
(43) Pub. Date: Jul. 8, 2004

(54) Novel dendritic polymers and their biomedical uses

(76) Inventors: Mark W. Grinstaff, Durnham, NC (US); Michael A. Carnahan, Durham, NC (US); Meredith T. Morgan, Durnham, NC (US); Kimberly A. Smeds, Springfield, VA (US); William C. Ray III, Durham, NC (US); Beth Walsh, Durham, NC (US)

Correspondence Address: NIXON & VANDERHYE, PC, 1100 N GLEBE ROAD, 8TH FLOOR, ARLINGTON, VA, 22201-4714, US

(21) Appl. No.: 10/468,902

(22) PCT Filed: Feb. 26, 2002
(86) PCT No.: PCT/US02/05638

(51) Int. Cl. A61K 48/00; C08G 63/48; C08G 63/91

(52) U.S. Cl. 424/78.3; 525/54.2

(57) ABSTRACT

Novel dendritic polymers are employed to clinically seal or repair wounds and treat traumatized or degenerative tissue. Novel crosslinkable biopolymers such as dendritic macromolecules are used in vitro, in vivo and in situ to treat ophthalmological, orthopaedic, cardiovascular, plastic surgery, pulmonary or urinary wounds or injuries. The crosslinkable dendritic macromolecules can be fabricated into cell scaffold/gel/matrix of specified shapes and sizes using one-photon and multi-photon spectroscopic techniques. The crosslinked polymers can be seeded with cells and used to repair or replace organs, tissues or bones. Alternatively, the polymers and cells can be mixed and injected into the in vivo site and crosslinked in situ for organ, tissue or bone repair or replacement.

### CROSS-REFERENCE TO RELATED APPLICATION

This application is based on, and claims priority benefits from, U.S. Provisional Application Serial No. 60/270,881 filed on Feb. 26, 2001, the entire content of which is expressly incorporated hereinto by reference.

### FIELD OF THE INVENTION

The present invention relates to clinical treatments, such as sealing or repairing wounds and the treatment of other traumatized or degenerative tissue. In particularly preferred forms, the present invention is specifically embodied in the use of novel crosslinkable biopolymers, such as dendritic macromolecules and their in vitro, in vivo, and in situ uses. Such uses include ophthalmological, orthopaedic, cardiovascular, pulmonary, or urinary wounds and injuries. These biomaterials/polymers are likely to be an effective sealant/glue for other surgical procedures where the site of the wound is not easily accessible or when sutureless surgery is desirable. Crosslinkable dendritic macromolecules can be fabricated into cell scaffold/gel/matrix of specified shapes and sizes using one-photo and multi-photon spectroscopic techniques. The polymers, after being crosslinked, can be seeded with cells and then used to repair or replace organs, tissue, or bones. Alternatively, the polymers and cells can be mixed and then injected into the in vivo site and crosslinked in situ for organ, tissue, or bone repair or replacement. The crosslinked polymers provide a three dimensional templates for new cell growth. This method can be used for a variety of reconstructive procedures, including custom molding of cell implants to reconstruct three dimensional tissue defects. Crosslinkable and non-crosslinkable biodendritic macromolecules can be used as drug delivery vehicles or carriers for pharmaceutical and medical imaging contrast agents.

### BACKGROUND AND SUMMARY OF THE INVENTION

A. Dendritic Macromolecules

Dendritic polymers are globular monodispersed polymers composed of repeated branching units emitting from a central core. (U.S. Pat. No. 5,714,166; U.S. Pat. No. 4,289,872; U.S. Pat. No. 4,435,548; U.S. Pat. No. 5,041,516; U.S. Pat. No. 5,362,843; U.S. Pat. No. 5,154,853; U.S. Pat. No. 5,739,256; U.S. Pat. No. 5,602,226; U.S. Pat. No. 5,514,764; Bosman, A. W.; Janssen, H. M.; Meijer, E. W. Chem. Rev. 1999, 99, 1665-1688. Fischer, M.; Vogtle, F. Angew. Chem. Int. Ed. 1999, 38, 884-905. Zeng, F.; Zimmerman, S. C. Chem. Rev. 1997, 97, 1681-1712. Tomalia, D. A.; Naylor, A. M.; Goddard, W. A. Angew. Chem. Int Ed. Engl. 1990, 29, 138.) These macromolecules are synthesized using either a divergent (from core to surface) (Buhleier, W.; Wehner, F. V.; Vogtle, F. Synthesis 1987, 155-158. Tomalia, D. A.; Baker, H.; Dewald, J.; Hall, M.; Kallos, G.; Martin, S.; Roeck, J.; Ryder, J.; Smith, P. Polymer Journal 1985, 17, 117-132. Tomalia, D. A.; Baker, H.; Dewald, J.; Hall, M.; Kallos, G.; Martin, S.; Roeck, J.; Ryder, J.; Smith, P. Macromolecules 1986, 19, 2466. Newkome, G. R.; Yao, Z.; Baker, G. R.; Gupta, V. K. J. Org. Chem. 1985, 50, 2003.)¹ or a convergent (from surface to core) (Hawker, C. J.; Frechet, J. M. J. J. Am. Chem. Soc. 1990, 112, 7638-7647) approach This research area has undergone tremendous growth in the last decade since the early work of Tomalia and Newkome. Compared to linear polymers, dendrimers are highly ordered, possess high surface area to volume ratios, and exhibit numerous end groups for functionalization. Consequently, dendrimers display several favorable physical properties for both industrial and biomedical applications including: small polydispersity indexes (PDI), low viscosities, high solubility and miscibility, and excellent adhesive properties. The majority of dendrimers investigated for biomedical/biotechnology applications (e.g., MRI, gene delivery, and cancer treatment) are derivatives of aromatic polyether or aliphatic amides and thus are not ideal for in vivo uses. (Service, R. F. Science 1995, 267, 458-459. Lindhorst, T. K.; Kieburg, C. Angew. Chem. Int. Ed. 1996, 35, 1953-1956. Ashton, P. R.; Boyd, S. E.; Brown, C. L.; Yayaraman, N.; Stoddart, J. F. Angew. Chem. Int. Ed. 1997, 1997, 732-735. Wiener, E. C.; Brechbeil, M. W.; Brothers, H.; Magin, R. L.; Gansow, O. A.; Tomalia, D. A.; Lauterbur, P. C. Magn. Reson. Med. 1994, 31, 1-8. Wiener, E. C.; Auteri, F. P.; Chen, J. W.; Brechbeil, M. W.; Gansow, O. A.; Schneider, D. S.; Beldford, R. L.; Clarkson, R. B.; Lauterbur, P. C. J. Am. Chem. Soc. 1996, 118, 7774-7782. Toth, E.; Pubanz, D.; Vauthey, S.; Helm, L.; Merbach, A. E. Chem. Eur. J. 1996, 2, 1607-1615. Adam, G. A.; Neuerburg, J.; Spuntrup, E.; Muhl;er, A.; Scherer, K.; Gunther, R. W. J. Magn. Reson. Imag. 1994, 4, 462-466. Bourne, M. W.; Margerun, L.; Hylton, N.; Campion, B.; Lai, J. J.; Dereugin, N.; Higgins, C. B. J. Magn. Reson. Imag. 1996, 6, 305-310. Miller, A. D. Angew. Chem. Int Ed. 1998, 37, 1768-1785. Kukowska-Latallo, J. F.; Bielinska, A. U.; Johnson, J.; Spinder, R.; Tomalia, D. A.; Baker, J. R. Proc. Natl. Aced. Sci. 1996, 93, 4897-4902. Hawthorne, M. F. Angew. Chem. Int. Ed. 1993, 32, 950-984. Qualmann, B.; Kessels M. M.; Musiol H.; Sierralta W. D.; Jungblut P. W.; L., M. Angew. Chem. Int. Ed. 1996, 35, 909-911). Biodendrimers are a novel class of dendritic macromolecules composed entirely of building blocks known to be biocompatible or degradable to natural metabolites in vivo. This patent describes the synthesis, characterization, and use of novel dendrimers and dendritic macromolecules called “biodendrimers or biodendritic macromolecules” composed of such biocompatible or natural metabolite monomers such as but not limited to glycerol, lactic acid, glycolic acid, succinic acid, ribose, adipic acid, malic acid, glucose, citric acid, etc. ¹Each cited patent and publication cited above and hereinafter is expressly incorporated into the subject application as if set forth fully therein.

The present invention is generally in the area of the synthesis and fabrication of dendritic polymers and copolymers of polyesters, polyethers, polyether-esters, and polyamino acids or combinations thereof. For example, poly(glycolic acid), poly(lactic acid), and their copolymers are synthetic polyesters that have been approved by the FDA for certain uses, and have been used successfully as sutures, drug delivery carriers, and tissue engineering scaffold for organ failure or tissue loss (Gilding and Reed, Polymer, 20:1459 (1979); Mooney et al., Cell Transpl., 2:203 (1994); and Lewis, D. H. in Biodegradable Polymers as Drug Delivery Systems, Chasin, M., and Langer, R., Eds., Marcel Dekker, New York, 1990). In tissue engineering applications, isolated cells or cell clusters are attached onto or embedded in a synthetic biodegradable polymer scaffold and this polymer-cell scaffold is next implanted into recipients (Langer and Vacanti, Science, 260:920 (1993). A large number of cell types have been used including cartilage cells (Freed et al., Bio/Technology, 12:689 (1994)). Like the novel biodendrimers described in this invention, the advantages include their degradability in the physiological environment to yield naturally occurring metabolic products and the ability to control their rate of degradation by varying the ratio of lactic acid. In the dendritic structures the degradation can be controlled by both the type of monomer used and the generation number.

A further embodiment of this invention is to attach biological recognition units for cell recognition to the end groups or within the dendrimer structure. For example the tripeptide arginine-glycine-aspartic (RGD), can be added to the structure for cell binding. Barrera et al. described the synthesis of a poly(lactic acid) (pLAL) containing a low concentration of N-epsilon.-carbobenzoxy-L-lysine units. The polymers were chemically modified through reaction of the lysine units to introduce arginine-glycine-aspartic acid peptide sequences or other growth factors to improve polymer-cell interactions (Barrera et al., J. Am. Chem. Soc., 115:11010 (1993); U.S. Pat. No. 5,399,665 to Bartera et al.). The greatest limitation in the copolymers developed by Barrera et al. is that only a limited number of lysine units can be incorporated into the backbone. In many tissue engineering applications, the concentration of biologically active molecules attached to the linear polymer is too low to produce the desired interactions between the polymer and the body. Consequently, there is a need for the development of optimal materials for use as scaffolds to support cell growth and tissue development in tissue engineering applications. In addition, there is a need for methods for introducing functionalities such as polyamino acids, peptides, carbohydrates into polyesters, polyether-esters, polycarbonates, etc. in order to improve the biocompatibility and other properties of the polymers. Furthermore there is a need for the development of polyester, polyether ester, polyester-amines, etc materials which include a sufficient concentration of derivatizable groups to permit the chemical modification of the polymer for different biomedical applications.

It is therefore an object of the invention to provide dendritic polymers and copolymers of polyesters and polyamino acids, polyethers, polyurethanes, polycarbonates, polyamino alcohols which can be chemically modified for different biomedical applications such as tissue engineering applications, wound management, contrast agents vehicles, drug delivery vechiles, etc. It is a further object of the invention to provide dendritic polymers and copolymers of polyesters and polyamino acids with improved properties such as biodegradability, biocompatibility, mechanical strength. It is still another object of the invention to provide dendritic polymers that can be derivatized to include functionalities such as peptide sequences or growth factors to improve the interaction of the polymer with cells, tissues, or bone.

The cellular response to conventional linear polymers including adhesion, growth, and/or differentiation of cells cannot be controlled or modified through changes in the polymer's structure, because these polymers do not possess functional groups, other than end groups, that permit chemical modification to change their properties, thereby limiting the applications of these polymers. Consequently the novel polymers described herein are substantially different.

B. Gels

The invention is generally in the area of using dendritic polymeric gels and gel-cell compositions in medical treatments. Gels are 3D polymeric materials which exhibit the ability to swell in water and to retain a fraction of water within the structure without dissolving. The physical properties exhibited by gels such as water content, sensitivity to environmental conditions (e.g., pH, temperature, solvent, stress), soft, adhesivity, and rubbery consistency are favorable for biomedical and biotechnological applications. Indeed, gels may be used as coatings (e.g. biosensors, catheters, and sutures), as “homogeneous” materials (e.g. contact lenses, burn dressings, and dentures), and as devices (e.g. artificial organs and drug delivery systems) (Peppas, N. A. Hydrogel in Medicine and Pharmacy, Vol I and II 1987. Wichterle, O.; Lim, D. Nature 1960, 185, 117-118. Ottenbrite, R. M.; Huang, S. J.; Park, K. Hydrogels and Biodegradable polymers for Bioapplications 1994; Vol. 627, pp 268).

Gel matrices for the entrapment of cells as artificial organs have been explored for more than fifteen years, and microencapsulation is a promising approach for a number of disease states including Parkinson's disease (L-dopamine cells), liver disease (hepatocyte cells), and diabetes (islets of Langerhans). In the past, for example, islets of Langerhans (the insulin producing cells of the pancreas) have beeb encapsulated in an ionically crosslinked alginate (a natural hydrogel) microcapsule with a poly-L-lysine coating, and successfully reduced blood sugar levels in diabetic mice following transplantation.

C. Macroporous Biodendritic Gels/Structures/Matrices

A current challenge in tissue engineering is the formation of well-ordered three-dimensional cell scaffolds. (R. P. Lanza, R. Langer, W. L. Chick, Principles of Tissue Engineering, R. G. Landes/Academic Press, San Diego, Calif., 1997. L. Christenson, A. G. Mikos, D. F. Gibbons, G. L. Picciolo, Tissue Eng. 3 (1997) 71. R. Langer, J. P. Vacanti, Science 260 (1993) 920. N. A. Peppas, R. Langer, Science 263 (1994) 1715. B. D. Ratner, A. S. Hoffman, F. J. Schoen, J. E. Lemons, Biomaterials Science: An Introduction to Material in Medicine, Academic Press, San Diego, 2000. Scientific American April (1999). Such temporary cell scaffolds are being explored as templates for reconstructed tissues by providing a site for cell attachment, proliferation, migration, and, in some instances, differentiation (J. Patrick, C. W., A. G. Mikos, L. V. McIntire, Frontiers in Tissue Engineering, Pergamon, N.Y., 1998). Biopolymers typically employed to construct cell scaffolds are linear macromolecules such as polyether glycols, and poly α-hydroxy acids (S. W. Shalaby, R. A. Johnson, Biomedical Polymers (1994) 2. M. Vert, S. M. Li, J. Mater. Sci. Mater. Med. 3 (1992) 432. E. J. Frazza, E. E. Schmitt, J. Biomed. Mater. Res. Symp. 1 (1971) 43.) Biodendritic polymers are amenable to processing methods for creating macroporous materials such as fiber bonding, (A. G. Mikos, Y. Bao, L. G. Cima, D. E. Ingber, C. A. Vacanti, R. Langer, J. Biomed. Mater. Res. 27 (1993) 183. D. J. Mooney, G. Organ, J. P. Vacanti, R. Langer, Cell Transplantation 3 (1994) 203.) solvent-casting and salt leaching, (A. G. Mikos, A. J. Thorsen, L. A. Czerwonka, Y. Bai, R. Langer, D. N. Wislow, J. P. Vacanti, Polymer 35 (1994) 1068) membrane lamination, (A. G. Mikos, G. Sarakinos, S. M. Leite, J. P. Vacanti, R. Langer, Biomater. 14 (1993) 323.) melt molding, (R. C. Thomson, M. J. Yaszemski, J. M. Powers, A. G. Mikos, J. Biomed. Sci. Polym. Ed. 7 (1995) 23) extrusion, M. S. Widmer, P. K. Gupta, L. Lu, R. K. Meszlenyi, G. R. D. Evans, K. Brandt, S. T. Gurelk, C. W. Patrick Jr, A. G. Mikos, Biomaterials 19 (1998) 1945) hydrocarbon templating, (V. P. Shastri, I. Martin, R. Langer, Proc. Natl. Acad. Sci. 97 (2000) 1970) and phase separation (H. Lo, M. S. Ponticiello, K. W. Leong, Tissue Eng. 1 (1995) 15).

A further embodiment of this invention is the use of crosslinkable biodendritic polymers in a templated-directed macroporous fabrication technique. This method has been applied to a variety of material science applications including separation and adsorbent media, catalytic supports, mechanical dampeners, and photonic crystals, but not to biomaterials. Typically, inorganic or polymeric materials are fabricated by controlled precipitation or polymerization in the presence of a sacrificial template (O. D. Velev, T. A. Jede, R. F. Lobo, A. M. Lenhoff, Nature 389 (1997) 447. A. A. Zakhidov, R. H. Baughman, Z. Iobal, C. Cui, I. Khayyrullin, O. Dantas, J. Marti, V. G. Ralchenko, Science 282 (1998) 897. J. E. G. J. Wijnhoven, W. L. Vos, Science 281 (1998) 802. S. A. Johnson, P. J. Ollivier, T. E. Mallouk, Science 283 (1999) 963. C. R. Martin, Science 266 (1994) 1961. S. H. Park, Y. Xia, Chem. Mater. 10 (1998) 1745) The template is then removed by calcination, hydrofluoric acid dissolution, or organic solvent dissolution to yield a macroporous material with voids reminiscent of the template (e.g, polystyrene beads). This technique offers several advantages including controlled pore size and density, monodisperse pore diameters, as well as room temperature processing with a wide range of polymers. Given these advantages for material fabrication, we adapted this approach to the processing of biopolymers and biodendritic macromolecules for tissue engineering scaffolds/gels/matrices.

A representative procedure is as follows. First, polystyrene beads of a desired size are initially isolated from aqueous suspension by centrifugation in an Eppendorf microfuge tube. Next the photocrosslinkable biopolymer and the photoinitiator (DMAP) are added (with a volume specific to the desired concentration) to the Eppendorf and mixed with the beads on a vortex spinner. The polymer is then photocrosslinked with an UV lamp and removed from the eppendorf tube. The crosslinked polymer containing the polystyrene beads is then submerged in toluene for approximately 72 hours to dissolve the beads. The macroporous biomaterials are then rinsed with copious amounts of ethanol and water and stored until further use.

Scanning electron micrographs of a series of different macroporous biomaterials show a honey-comb structures produced from a cubic closed packed arrangement of the polystyrene beads in the biopolymer prior to photocrosslinking and bead dissolution. By changing the polystyrene bead/biopolymer ratio, different macroporous structures can be produced. We have also recently prepared macroporous biomaterials using 0.2 to 90 micron polystyrene templates as well as with a photocrosslinkable polysaccharide (hyaluronic add). The size of the pores correlates with the size of the sacrificial template, and are uniform throughout the structures.

In summary, a mild procedure for forming well-ordered macroporous biomaterials is described. As demonstrated in the above examples, the advantages of this technique include: 1) controlled pore sizes from ˜0.2 to 90 microns, 2) controlled pore density from 0.1 g to 1.0 g/mL, 3) monodisperse pore diameters, 4) interconnected porous structures, and 5) mild room temperature processing

The photocrosslinkable biodendrimers synthesized are also amenable to standard photolithography processing methods as demonstrated by construction of a simple line pattern (100 microns) using a mask. Atomic force microscopy (AFM) shows the film to be smooth and uniform with no appreciable defects at 50 nm resolution. The RMS average of height deviation is approximately 1.5 nm.

A further embodiment of this invention is microstructure fabrication procedures using light, photoinitiators and photocrosslinkable biopolymers/biodendritic macromolecules. Photopolymerization can occur via a single- or multi-photon process. In two-photon polymerization, laser excitation of a photoinitiator proceeds through at least one virtual or non-stationary state (S. Maruo, O. Nakamura, S. Kawata, Opt. Lett. 22 (1997)132. J. D. Pitts, P. J. Campagnola, G. A. Epling, S. L. Goodman, Macromolecules 33 (2000)1514). The photo-initiator will absorb two near-IR photons, driving it into the S₂ state, followed by decay to the SI and intersystem crossing to the long-lived triplet state. When the spatial density of the incident photons is high, the initiator molecule (in the triplet state) will abstract an electron from TEA thus start the photocrosslinking reaction of the polymer to create the scaffold. Importantly, complex and detailed structures may be fabricated with high precision since 2-photon absorption is extremely localized under narrow focusing conditions. Controlled microfabrication via 2-photon-induced polymerization (TPIP) has been used to develop 3-dimensional structures from photopolymerizable resins for use as photonic band gap materials and semiconductors (S. M. Kirkpatrick, J. W. Baur, C. M. Clark, L. R. Denny, D. W. Tomlin, B. R. Reinhardt, R. Kannan, M. O. Stone, Appl. Physics. A. 69 (1999) 461). In accordance with the present invention, TPIP is applied towards the synthesis of biomedically useful structures from a solution of biopolymers to demonstrate this method for ultimately creating well-defined three-dimensional tissue engineering scaffolds using our novel photocrosslinkable biodendrimers.

TPIP is performed using the following system. Specifically, a femtosecond near-IR titanium sapphire laser (Coherent 900° F.) coupled to a laser scanning confocal microscope is employed. The set-up is diagrammed in Figure. The average power and wavelength used for TPIP are 50 mW and 780 nm, respectively. The microscope is equipped with scanning mirrors for point and raster scans. Approximately 20 μL of solution are dropped onto a glass microscope slide before loading onto the microscope stage for laser irradiation.

Pitts et al., examined TPIP of BSA and fibrinogen [Pitts, 2000 #438], but these polymers do not have a high density of photocrosslinking groups. Aqueous mixtures were first prepared of acrylate-terminated biopolymer, initiator, and co-initiator, with a concentration ratio of 10000:1000:1. Eosin Y (EY) was used as initiator and triethanolamine (TEA) was used as a co-initiator. A simple line pattern was constructed. Light microscopy, scanning electron microscopy, and atomic force microscopy confirmed the fabricated structures.

Besides covalently crosslinked gels/matrices/scaffolds, the invention describes end groups for self assembly via hydrogen bond or ionic charge networks. The first example, uses one biodendrimer functionalized with lysine and a second with succinic acid. Upon mixing at pH=7.4, the two biodendrimers will self assemble and form a gel. Likewise, it is proposed to use hydrogen bonding networks present in DNA, for example, a G:C base pair. These G/C derivatized dendrimers can be synthesized using the same nucleoside starting materials used to prepare PNAs.

The present invention also proposes to use peptide hydrogen bonding interaction to form a gel. Silk is a natural polypeptide composed primarily of repeating Gly-Ala units. These peptides form long antiparallel sheets with strong hydrogen bond interactions between the neighboring amide proton and carbonyl. By attaching these peptides to the ends of the biodendrimer a three-dimensional crosslinked gel is expected to form. Using principles based upon non-covalent interactions, macroscopic gels composed of biodendrimers can be created.

D. Dendritic Cell Constructs/Scaffolds/Matrices/Gels for Organ/Tissue Repair or Replacement

The present invention is also generally employed in the area of using dendritic polymeric-cell compositions in medical treatments. Several useful examples, which are not to be construed as limiting the present invention, are described below.

Craniofacial contour deformities. Craniofacial contour deformities currently require invasive surgical techniques for correction. These traumatic or congenital deformities are often severe. Alternatively, surgery is requested for an aesthetic personal viewpoint. These deformities often require augmentation in the form of alloplastic prostheses which suffer from problems of infection and extrusion. A minimally invasive method of delivering additional autogenous cartilage or bone to the craniofacial skeleton would minimize surgical trauma and eliminate the need for alloplastic prostheses. By injecting a crosslinkable gel and cells (autoglous or otherwise) one could augment the craniofacial osteo-cartilaginous skeleton with autogenous tissue, without extensive surgery. An embodiment of this inventionis the use of biodendritic cell compositions for treating craniofacial contour deformities.

Breast Tissue Repair of Auqmentation. Mammary glands are modified sweat glands attached to the underlying muscle of the anterior chest wall by a layer of connective tissue. A single mammary gland consists of 15-25 lobes, separated by dense connective tissue formed primarily by fibroblasts and bundles of collagen fibers, and adipose tissue containing adipose (fat) cells held together by reticular and collagen fibers. A lactiferous duct that branches extensively is within each lobe. Glandular epithelial cells (alveolar cells) that synthesize and secrete milk into the duct system are located at the ends of the smallest branches. The ducts are composed of simple cuboidal and columnar epithelium. The alveolar cells are embedded in loose connective tissue containing collagen fibers and fibroblasts, lymphocytes, and plasma cells. Close to the alveolar and duct epithelial cells are myoepithelial cells which respond to hormonal and neural stimuli by contracting and expressing the milk. Each lactiferous duct opens onto the surface of the breast through the skin covering the nipple.

Breast surgery can be broadly categorized as either cosmetic or therapeutic. Cosmetic surgeries include augmentation using implants, reduction or reconstruction. Therapeutic surgery is the primary treatment for most early cancers and includes 1) radical surgery that may involve removal of the entire soft tissue anterior chest wall and lymph nodes and vessels extending into the head and neck, 2) lumpectomy, which may involve only a small portion of the breast; and 3) laser surgery for destruction of small regions of tissue. Often reconstructive surgery with implants is used in radical breast surgery. The radical mastectomy involves removal of the breast, both the major and minor pectoralis muscles, and lymph nodes.

Presently, more than 250,000 reconstructive procedures are performed annually, and there are few alternatives to reconstruction as a result of breast cancer, congenital defects, or damage from trauma. Breast reconstruction is frequently used at the time of, or just after, mastectomy for cancer. Reconstructive procedures frequently involve moving vascularized skin flaps with underlying connective and adipose tissue from one region of the body to another. There are numerous surgical methods of breast reconstruction, including tissue expansion followed by silicone implantation, latissimus dorsi flap, pedicled transversus abdominis myocutaneous flap (TRAM), free TRAM flap, and free gluteal flap. Full reconstruction often requires additional procedures over mastectomy and primary reconstruction. These procedures include tissue-expander exchange for permanent implant, revision of reconstruction, nipple reconstruction, and mastopexy/reduction.

Silicone prosthesis that are frequenity used for reconstruction and augmentation, have afforded many medical complications. It is desirable to have an alternative material for implantation that functions properly, looks and feels like normal tissue, and does not interfere with X-ray diagnosis. It is therefore an object of the invention to provide methods and compositions for reconstruction and augmentation of breast tissue using dendritic polymers or dendritic macromolecules and cell constructs.

Oral tissue repair Oral tissue repair is another area where three-dimensional polymer scaffold/matrices/gels can be used for proliferating oral tissue cells and the formation of components of oral tissues analogous to counterparts found in vivo. These proliferating cells produce proteins, secrete extracellular matrix components, growth factors and regulatory factors necessary to support the long term proliferation of oral tissue cells seeded on the matrix. The production of the fibrous or stromal extracellular matrix tissue that is deposited on the matrix is conducive for the long term growth of the oral tissues in vitro. The three-dimensionality of the scaffold/matrices/gels more closely approximates the conditions in vivo for the particular oral tissues, allowing for the formation of microenvironments encouraging cellular maturation and migration. Specific growth or regulatory factors can also be added to further enhance cell growth and extracellular matrix production.

Tissues of interest include dental pulp, dentin, gingival, submucosa, cementum, periodontal, oral submucosa or tongue tissue cells. The tissue sample subsequently formed is a dental pulp, dentin, gingival submucosa, cementum, periodontal, oral submucosa or tongue tissue sample. The tissue sample may be formed by culturing viable starting cells obtained from an oral tissue sample enriched in dental pulp-derived fibroblasts. In certain aspects of the invention the viable starting cells enriched in dental pulp-derived fibroblasts are obtained from an extracted tooth. Additionally, the tissue sample may be formed by culturing viable starting cells obtained from an oral tissue sample enriched in gingival submucosal fibroblasts, pulp or periodontal ligament fibroblasts as a source of cells. Gingival biopsies are obtainable by routine dental procedures with little or no attendant donor site morbidity. An embodiment of this invention is the use of biodendritic cell compositions for treating oral repair.

It will be understood that the oral tissue sample may again be separated from the matrix prior to application to the patient, or placed in vivo and crosslinked in situ. Equally, the oral tissue sample may be applied in combination with the matrix, wherein the matrix would preferably be a biocompatible matrix. Implantation of a cultured matrix-cell preparation into a specific oral tissue site of an animal to effect reconstruction of oral tissue may involve a biodegradable matrix or a non-biodegradable matrix, depending on the intended function of the preparation.

Urinary incontinence. Urinary incontinence is the most common and the most intractable of all GU maladies. The inability to retain urine and not void urine involuntarily is controlled by the interaction between two sets of muscles. The detrusor muscle, a complex of longitudinal fibers forming the external muscular coating of the bladder, activates the parasympathetic nerves. The second muscle, which is a smooth/striated muscle of the bladder sphincter, and the act of voiding requires the sphincter muscle be voluntarily relaxed at the same time that the detrusor muscle contracts. As one ages, the ability to voluntarily control the sphincter muscle deteriorates. The most common incontinence, particular in the elderly, is urge incontinence where there is only a brief warning before immediate urination. Urge incontinence is a result by a hyperactive detrusor and is typicaly treated with medication and/or “toilet training”. However, reflex incontinence occurs without warning and is usually the result of an impairment of the parasympathetic nerve system. The common incontinence found in elderly women is stress incontinence, which is also observed in pregnant women. This type of incontinence accounts for over half of the total number of cases. Stress incontinence occurs under conditions such as sneezing, laughing or physical effort and is characterized by urine leaking. There are five recognized categories of severity of stress incontinence, designated as types as 0, 1, 2a, 2b, and 3. Type 3 is the most severe and requires a diagnosis of intrinsic sphincter deficiency or ISD (Contemporary Urology, March 1993). There are several treatments including medication, weight loss, exercise, and surgical intervention. The two most common surgical procedures involve either elevating the bladder neck to counteract leakage or constructing a lining from the patient's own body tissue or a prosthetic material such as PTFE to put pressure on the urethra. The second option is to use prosthetic devices such as artificial sphincters to external devices such as intravaginal balloons or penile clamps. The above methods of treatment are very effective for periods typically more than a year. Overflow incontinence is caused by anatomical obstructions in the bladder or underactive detrustors. An embodiment of this invention is the use of biodendritic cell compositions for treating urinary incontinence.

Organ transplantation A cell-scaffold/gel/matrix composition is prepared for in situ polymerization or in vitro use for subsequent implanting to produce functional organ tissue in vivo. The scaffold/gel/matrix is three-dimensional and is composed of crosslinked (covalent, ionic, hydrogen-bondned, etc.) dendritic polymer or copolymer. The scaffold can also be formed from fibers of the dendritic polymer. The cells used are derived from vascularized organ tissue or stem cells and are then suspended in the polymer and subsequently injected in vivo and photocrosslinked to form the gel-cell composite. Alternatively, the cell are attached in vitro to the surface of the preformed crosslinked scaffold or gel to produce functional vascularized organ tissue in vivo. The scaffold/gel/matrix can also be partially chemically degraded with base or acid washings to afford a more hydrophilic material. It is a further embodiment of this invention to separate the linear/dendritic fibers of the woven scaffold by a distance over which diffusion of nutrients and gases can occur typically between 100 and 300 microns. Alternatively, a macroporous gel can be produced by a template, foaming, etc. procedure as described in this invention whereby the uniform or non-uniform pores of 1 to 1000 microns are formed. These gel/scaffold/matrix structures provides for the diffusion and exchange of nutrients, gases, and waste to and from cells proliferating throughout the scaffold in an amount effective to maintain cell viability throughout the material in the absence of vascularization.

Cells attached to the gel/scaffold/matrix may be lymphatic vessel cells, pancreatic islet cells, hepatocytes, bone forming cells, muscle cells, intestinal cells, kidney cells, blood vessel cells, thyroid cells or cells, of the adrenal-hypothalamic pituitary axis. Besides these types of cells, stem cells can be used that subsequently convert to a desired specific cell type.

For example, diabetes mellitus is a disease caused by loss of pancreatic function. Specifically, the insulin producing beta cells of the pancreas are destroyed and thus serum glucose levels rise to high values. As a result, major problems develop in all systems secondary to the vascular changes. Diabetes is estimated to afflict more than 16,000,000 individuals in the United States. Sadly, this number is growing at an alarming rate of about 600,000 new cases diagnosed every year. Presently, diabetes is the third largest cause of death in the U.S., primarily from micro- and macrovascular complications. These complications include limb amputations, ulceration, vascular damage, kidney failure, strokes, and heart attacks which are a result. The daily injection of insulin was once thought to be an effective treatment for diabetes. However, for individuals who have insulin dependent diabetes mellitus (IDDM) and undergo traditional insulin therapy, these horrific complications still persist. In 1992, the Diabetes Control and Complications Trial (DCCT) reported that tightly regulated glucose reduces the risk of these complications. Yet, intensive insulin treatment is not entirely safe due to increased incidences of hypoglycemic episodes. Eastman and Gordon writing on the implications of the DCCT for diabetes treatment stated “the success of intensive treatment as done in the DCCT is both a triumph and a challenge for the health care system: a triumph because we now know that metabolic control matters, and a challenge because the results were achieved by an integrated team of health care researchers with expertise in medicine, education, nutrition, diabetes, self-management skills and human behavior.” These teams are not and probably will not be available in the future for the treatment of the vast majority of patients with diabetes. Consequently, there is a need for novel technologies such as those described in his invention that will provide normal regulation of blood glucose.

The current method of treatment available to diabetic is exogenous administration of insulin, on a regular basis. However, this treatment still results in imperfect control of blood sugar levels. The experimental approach of whole pancreatic tissue transplantation is high risk. However there is not sufficient number of donor pancreases available for diabetics. After transplantation, the serum glucose appears to be controlled in a more physiological manner. This approach is far better then the transplantation of isolated islet cells themselves. An improvement in recent years, has been the encapsulation of the cells to prevent an immune attack by the host. There is evidence of short term function, but the long term results have been less than satisfactory (D. E. R. Sutherland, Diabetologia 20, 161-18 (1981); D. E. R. Sutherland, Diabetologia 20, 435-500 (1981)). Thus whole organ pancreatic transplantation is the preferred treatment. A further embodiment of this invention is to encapsulate/embed islet cells in a biodendritic crosslinkable polymer and subsequent transplantation in the host.

Another useful application of said biodendritic polymers is in the treatment of hepatic failure. Hepatic failure arises as a result of scaring due to a disease, genetic irregularitites, or from injury. Transplantation is the current solution, and without such treatment the outcome is death. It is estimated that 30,000 people die of hepatic failure every year in the United States, with a cost to society of approximately $14 billion annually.

The indications for a liver transplantation include for example acute fulminant hepatic failure, chronic active hepatitis, biliary atresia, idiopathic cirrhosis, primary biliary cirrhosis, sclerosing cholangitis, inborn errors of metabolism, and some types of malignancy. The current method of treatment involves maintaining the patient until a liver becomes available for transplantation. Transplantation of the whole liver is an increasingly successful surgical manipulation. However, the technical complexity of the surgery, the enormous loss of blood, the postoperative conditions, and expense of the operation make this procedure only available in major medical centers. Given the scarcity of the donor organs, the needs of the patient will not be satisfied, Unfortunately, 30,000 patients die each year of end-stage liver disease. Good artificial hepatic support for patients awaiting transplantation is not widely available. Patients suffering from alcohol-induced liver disease represent another large group of patients awaiting treatment. Today patients with end-stage liver disease as a result of alcohol consumption do not have access to transplantation, since there is a scarcity of donor organs and current healthcare compliances. The mortality rates for cirrhosis vary greatly from country to country, ranging from 7.5 per 100,000 in Finland to 57.2 per 100,000 in France. In the U.S., there has been a 70% increase in the number of deaths over the last 25 years. Furthermore, the morbidity for liver cirrhosis is twenty-eight times higher among serious problem drinkers than among nondrinkers.

The liver and pancreas are not the only vital organ systems for which there is inadequate treatment in the form of replacement or restoration of lost function. For example, loss of the majority of the intestine was a fatal condition in the past. Although patients can now survive with intravenous nutrition supplied via the veins, this is an inadequate approach since many complications arise during care. Patients on total parenteral nutrition can develop fatal liver disease or can develop severe blood stream infections. Intestinal transplantation is not a current option since a large number of lymphocytes in the donor intestine are transferred to the recipients. This affords an immunologic reaction “graft vs. host” disease, in which the lymphocytes from the transplanted intestine attack. This eventually leads to death. A further embodiment of this invention is to use biodendritic crosslinkable polymer treating organ loss or repair.

Diseases of the heart and muscle are also a major cause of morbidity and mortality in the world. Cardiac transplantation has been an increasingly successful technique, but, as in the case of liver transplants, requires immunosuppressant drugs and a donor heart. Although organ transplantation is a current remedy for many indications, the scarcity of donor tissue has increased. For example, only a small number of donors are available in the U.S. for the 800-1,000 children/year who need a liver transplantation. Transplantation is often associated with 1) recipients who are very ill and thus the likelihood for success is diminished 2) a complex surgical procedure typically associated with blood loss, 3) the need for a rapid operation since the preservation time is short. The transplantation of only those parenchymal elements necessary to replace lost function has been proposed as an alternative to whole or partial organ transplantation (P. S. Russell, Ann. Surg. 201 (3), 255-262 (1985)). This approach has several attractive features, including avoiding major surgery with its attendant blood loss, anesthetic difficulties, and complications. Since only those cells which supply the needed function are replaced, the problems with passenger leukocytes, antigen presenting cells, and other cell types which may promote the rejection process may be reduced or even avoided. Using this approach, the possibility to use cells in an autotransplantation procedure is possible with cells of the recipient's expanded in culture or stem cells that have differentiated to a specific cell type. For example, Demetriou et al reported successful implantation of hepatocytes attached to collagen coated microcarrier beads (A. A. Demetriou, et al., Science 233, 1190-1192 (1986)). A further embodiment of this invention is to use biodendritic crosslinkable polymer for organ transplantation.

Skin is another organ that can be damaged by disease or injury. Skin plays a vital role of protecting the body from fluid loss and disease. Skin grafts have been prepared previously from animal skin or the patient's skin, more recently “artificial skin” formed by culturing epidermal cells. In U.S. Pat. No. 4,485,097 Bell discloses a skin-equivalent material composed of a hydrated collagen lattice with platelets and fibroblasts and cells such as keratinocytes. U.S. Pat. No. 4,060,081, to Yannas et al. discloses a multilayer membrane useful as synthetic skin formed from an insoluble non-immunogenic and a non-toxic material such as a synthetic polymer for controlling the moisture flux of the overall membrane. In U.S. Pat. No. 4,458,678, Yannas et al. describe a process for making a skin-equivalent material wherein a fibrous lattice formed from collagen cross-linked with glycosaminoglycan is seeded with epidermal cells. A disadvantage to the first two methods is that the matrix is formed from a “permanent” synthetic polymer. In fact, the limitations of this material are discussed in the authors article published in 1980 (Yannas and Burke J. Biomed. Mater. Res., 14, 65-81 (1980)).

Examples of cells that are suitable for use in this invention include but are not limited to hepatocytes and bile duct cells, islet cells of the pancreas, parathyroid cells, thyroid cells, cells of the adrenal-hypothalmic-pituitary axis including hormone-producing gonadal cells, epithelial cells, nerve cells, heart muscle cells, blood vessel cells, lymphatic vessel cells, kidney cells, and intestinal cells, cells forming bone and cartilage, smooth and skeletal muscle.

It is a further object of the invention to provide a method and means for designing, constructing, and utilizing artificial dendritic matrices as temporary scaffolding for cellular growth and implantation. A further embodiment of the invention to provide biodegradable, non-toxic matrices which can be utilized for cell growth, both in vitro, in vivo, and in situ. The cell scaffold/matrix/gel can be formed in vitro or in situ by crosslinking. It is another object of the present invention to provide a method for configuring and constructing biodegradable artificial matrices such that they not only provide a support for cell growth but allow and enhance vascularization and differentiation of the growing cell mass following implantation. It is yet another object of the invention to provide matrices in different configurations so that cell behavior and interaction with other cells, cell substrates, and molecular signals can be studied in vitro.

Polymeric matrix can be used to seed cells and subsequently implanted to form a cartilaginous structure, as described in U.S. Pat. No. 5,041,138 to Vacanti, et al., but this requires surgical implantation of the matrix and shaping of the matrix prior to implantation to form a desired anatomical structure. Hubbell (U.S. Pat. No. 1,995,000478690) describes linear crosslinkable polymers for mixing with cells, followed by in vivo injection and in situ polymerization, however the polymers are nondendritic structures that lack greater optimization of degradation, crosslinking, and chemical and biological derivitazation.

E. Tissue Sealants

The dendritic macromolecules of the present invention are also usefully employed as a tissue sealant. This biomaterial is likely to be an effective sealant/glue for other surgical procedures (e.g., leaking blebs, nephrotomy closure, bronchopleural fistula repair, peptic ulcer repair, tympanic membrane perforation repair, etc.) where the site of the wound is not easily accessible or when sutureless surgery is desirable.

Cornea perforation treatment: Corneal perforations afflict a fraction of the population and are produced by a variety of medical conditions (e.g., infection, inflammation, xerosis, neurotrophication, and degeneration) and traumas (chemical, thermal, surgical, and penetrating). Unfortunately, corneal perforations often lead to loss of vision and a decrease in an individual's quality of life. Depending on the type and the origin of the perforation, different treatments are currently available from suturing the wound to a cornea graft. However, this is a difficult surgical procedure given the delicate composition of the cornea and the severity of the wound which increase the likelihood for leakage and severe astigmatism after surgery. In certain cases, perforations that cannot be treated by standard suture procedures, tissue adhesives (glues) are used to repair the wound. This type of treatment is becoming very attractive because the method is the simplest, quickest and safest, and corresponds to the requirement of a quick restoration of the integrity of the globe to avoid further complications. Besides an easy and fast application on the wound, the criteria for an adhesive are to 1) bind to the tissue (necrosed or not, very often wet) with an adequate adhesion force, 2) be non-toxic, 3) be biodegradable or resorbable, 4) be sterilizable and 5) not interfere with the healing process. Various alkyl-cyanoacrylates are available for the repair of small perforations. However, these “super glues” present major inconveniences. Their monomers, in particular those with short alkyl chains, might be toxic. They also polymerize too quickly leading to applications that might be difficult and, once polymerized, the surface of the glue is rough and hard which leads to patient discomfort and a need to wear contact lens. Even though cyanoacrylate is tolerated as a corneal sealant, a number of complications have been reported including cataract formation, corneal infiltration, glaucoma, giant papillary conjunctivitis, and symblepharon formation. Furthermore, in more than 60% of the patients, additional surgical intervention was needed.

Other glues have also been developed. Adhesive hemostats, based on fibrin, are usually constituted of fibrinogen, thrombin and factor XIII. Systems with fibrinogen and photosensitizers activated with light are also being tested. If adhesive hemostats have intrinsic properties which meet the requirements for a tissue adhesive, autologous products (time consuming in an emergency) or severe treatments before clinical use are needed to avoid any contamination to the patient. An ideal sealant for corneal perforations should 1) not impair normal vision, 2) quickly restore the intraocular pressure, IOP, 3) maintain the structural integrity of the eye, 4) promote healing, 5) adhere to moist tissue surfaces, 6) possess solute diffusion properties which are molecular weight dependent and favorable for normal cornea function, 7) possess Theological properties that allow for controlled placement of the polymer on the wound, and 8) polymerize under mild conditions. A further embodiment of this invention is to use biodendritic crosslinkable polymers for sealing corneal perforations.

Retinal holes: Techniques commonly used for the treatment of retinal holes such as cryotherapy, diathermy and photocoagulation are unsuccessful in the case of complicated retinal detachment, mainly because of the delay in the application and the weak strength of the chorioretinal adhesion. Cyanoacrylate retinopexy has been used in special cases. It has also been demonstrated that the chorioretinal adhesion is stronger and lasts longer than the earlier techniques. As noted previously with regard to corneal perforation treatment, the extremely rapid polymerization of cyanoacrylate glues (for example, risk of adhesion of the injector to the retina), the difficulty to use them in aqueous conditions and the toxicity are inconveniences and risks associated with this method. The polymerization can be slowed down by adding iophendylate to the monomers but still the reaction occurs in two to three seconds. Risks of retinal tear at the edge of the treated hole can also be observed because of the hardness of cyanoacrylate once polymerized. A further embodiment of this invention is to use biodendritic crosslinkable polymer for sealing retinal holes.

Leaking blebs: Leaking filtering blebs after glaucoma surgery are difficult to manage and can lead to serious, vision-threatening complications. Leaking blebs can result in hypotony and shallowing of the anterior chamber, choroidal effusion, maculopathy, retinal, and choroidal folds, suprachoroidal hemorrhage, corneal decompensation, peripheral anterior synechiae, and cataract formation. A leaking bleb can also lead to the loss of bleb function and to the severe complications of endophthalmaitis. The incidence of bleb leaks increases with the use of antimetabolites. Bleb leaks in eyes treated with 5-fluorouracil or mitomycin C may occur in as many as 20 to 40% of patients. Bleb leaks in eyes treated with antimetabolities may be difficult to heal because of thin avascular tissue and because of abnormal fibrovascular response. If the leak persists despite the use of conservative management, a 9-0 to 10-0 nylon or absorbable suture on a tapered vascular needle can be used to close the conjunctival wound. In a thin-walled or avascular bleb, a suture may not be advisable because it could tear the tissue and cause a larger leak. Fibrin adhesives have been used to close bleb leaks. The adhesive is applied to conjunctival wound simultaneously with thrombin to form a fibrin clot at the application site. The operative field must be dry during the application because fibrin will not adhere to wet tissue. Cyanoacrylate glue may be used to close a conjuctival opening. To apply the glue, the surrounding tissue must be dried and a single drop of the cyanoacrylate is placed. The operative must be careful not to seal the applicator to the tissue or to seal surrounding tissue with glue given its quick reaction. A soft contact lens is then applied over the glue to decrease patient discomfort. However this procedure can actually worsen the problem if the cyanoacrylate tears from the bleb and causes a larger wound. A further embodiment of this invention is to use biodendritic crosslinkable polymers for sealing leaking blebs.

Corneal transplants: In a corneal transplant the surgeon makes approximately 16 sutures around the transplant to secure the new cornea in place. A sutureless procedure would therefore be highly desirable and would offer the following advantages: (1) sutures provide a site for infection, (2) the sutured cornea takes 3 months to heal before the sutures need to be removed, and (3) the strain applied to the new cornea tissue from the sutures can distort the cornea. A further embodiment of this invention is to use biodendritic crosslinkable polymers for sealing a corneal transplant.

Endocapsular lens replacement: Cataract is an opacity of the lens mainly due to the natural aging of the eye and some diseases. Edema, protein denaturation of the lens fibers and necrosis create opaque zones that can lead to blindness. Total lens extraction is infrequently performed today. This traumatic surgery has been replaced by aspiration of the nucleus and the cortex of the lens after their fragmentation by ultrasound and aspiration. Then an implant is inserted into the capsular bag. The first polymeric matrix, used for more than 50 years, was the poly(methylmethacrylate) (PMMA) as lens replacement or intracapsular bag implant. Silicone and hydrogels that can be implanted in the capsular bag through a smaller incision than the one made for rigid implants have been developed. One of the main issues, beside the biocompatibility of the material, is the mechanical dislocation of the implant. Depending on the material, the implantation site and the surgical techniques, different designs of implants can be found. Silicone is, for example, injected in an inflatable thin silicone membrane previously implanted in the capsular bag.

An artificial lens composed of hydrogels in concentric annular rings with different radii of curvature has been proposed in U.S. Pat. No. 4,906,246. Furthermore, the injection of prepolymers such as urethanes, polypropylene glycols, polybutylene glycols and silicones that can be cross-linked by irradiation in the presence of photoinitiators such as aryl ketones have been disclosed in U.S. Pat. No. 4,919,151. The molecular weight and the crosslinking degree of the polymers can be modified to allow for a suitable refractive index. However, the biocompatibility of these systems has not been demonstrated.

Besides ophthalmological applications these photocrosslinkable polymers have additional surgical uses when the site of the wound is not easily accessible or when sutureless surgery is desired. These photopolymerizable sealants/glues may be of potential use for urinary tract surgery (nephrotomy closure, urethral repair, hypospadia repair), pulmonary surgery (sealing parenchymal & bronchial leaks, bronchopleural fistula repair, persistent air leak repairs), G.I. tract and stomach surgery (parotid cutaneous fistula, tracheo-oesophageal fistula, peptic ulcer repair), joint surgery (cartilage repair, meniscal repair), heart surgery (cardiac ventricular rupture repair), brain surgery (dural defect repairs), ear surgery (ear drum perforation), and post-surgical drainage reduction (mastectomy, axillary dissection). The ease of application, as well as the ability to quickly and precisely seal a wet or dry wound, means that this material may prove to be superior to the previous glues used in many of the above applications

F. Wound Dressings

In the majority of the cases, the treatment used for wound closure is the classical suture technique. However, depending on the type, the origin of the wound as well as the location of the patient, the use of tissue adhesives (e.g., glues, sealants, patches, films and the like is an attractive alternative to the use of sutures. Beside an easy and fast application on the wound, the criteria for an adhesive are to bind to the tissue (necrosed or not, sometimes wet) with an adequate adhesion force, to be non-toxic, biodegradable or resorbable, sterilizable, selectively permeable to gases, impermeable to bacteria and able to control evaporative water loss. Finally, the two main properties of the adhesive are to protect the wound and to enhance the healing process or at least not prevent it. Numerous sealants have been investigated and used for different clinical applications.

Adhesive hemostats, based on fibrin, are the most common products of biological origin. These sealants are usually constituted of fibrinogen, thrombin and factor XIII, as well as fibrinogen/photosensitizers systems. If their intrinsic properties meet the requirements for a tissue adhesive, autologous products (which are time consuming in emergency) or severe treatments before clinical use are needed to avoid any contamination to the patient.

Synthetic materials, mainly polymers and hydrogels in particular have been developed for wound closure. Alkyl-cyanoacrylates are available for the repair of cornea perforations. One investigator has observed no difference in healed skin incisions that were treated by suture or by ethyl-2-cyanoacrylate-“Mediglue” application. However, these “super glues” present major inconveniences. Their monomers, in particular those with short alkyl chains, are or might be toxic and they polymerize too quickly leading to difficulty in treating the wound. Once polymerized, the surface of the glue is rough and hard. This might involve discomfort to the patient and, for example, in case of cornea perforation treatment, a contact lens needs to be worn. Other materials have been commercialized such as “Biobrane II” (composite of polydimethylsiloxane on nylon fabric) and “Opsite” (polyurethane layer with vinyl ether coating on one side). A new polymeric hemostat (poly-N-acetyl glucosamine) has been studied for biomedical applications such as treatment of gastric varices in order to replace cyanoacrylate (voumakis). Adhesives based on modified gelatin are also found to treat skin wounds. Photopolymerizable poly(ethylene glycol) substituted with lactate and acrylate groups are used to seal air leaks in lung surgery.

G. Prevention of Adhesions

Yet another aspect of the invention provides a method for preventing the formation of adhesions between injured tissues by inserting a barrier composed of a biodendritic polymer or combinations of linear and biodendritic polymers between the injured tissues. This polymeric barrier acts as a sheet or coating on the exposed injured tissue to prevent surgical adhesions (Urry et al., Mat. Res. Soc. Symp. Proc., 292, 253-64 (1993). This polymeric barrier will dissolve over a time course that allows for normal healing to occur without formation of adhesions/scars etc. Adhesion formation is a major post-surgical complication. Today, the incidence of clinically significant adhesion is about 5 to 10 percent with some cases cases as high as 100 percent. Among the most common complications of adhesion formation are obstruction, infertility, and pain. Occasionally, adhesion formation requries a second operative procedure to remove adhesion, further complicating the treatment. Given the wide-spread occurrence of post-surgical adhesions, a number of approaches have been explored for preventing adhesions (Stangel et al., “Formation and Prevention of Postoperative Abdominal Adhesions”, The Journal of Reproductive Medicine, Vol. 29, No. 3, March 1984 (pp. 143-156), and dizerega, “The Cause and Prevention of Postsurgical Adhesions”, published by Pregnancy Research Branch, National Institute of Child Health and Human Development, National Institutes of Health, Building 18, Room 101, Bethesda, Md. 20205.)

A number of procedures have been explored for prevention of post-surgical adhesion including 1) Systemic administration of ibuprofen (e.g., see Singer, U.S. Pat. No. 4,346,108), 2) Parenteral administration of antihistamines, corticosteroids, and antibiotics, 3) Intraperitoneal administration of dextran solution and of polyvinylpyrrolidone solution, 4) Systemic administration of oxyphenbutazone, a non-steroidal anti-inflammatory drug that acts by inhibiting prostaglandin production, and 5) Administration of linear synthetic and natural polymers (Hubell 6060582; Fertil. Steril., 49:1066; Steinleitner et al. (1991) “Poloxamer 407 as an Intraperitoneal Barrier Material for the Prevention of Postsurgical Adhesion Formation and Reformation in Rodent Models for Reproductive Surgery,” Obstetrics and Gynecology, 77(1):48 and Leach et al. (1990) “Reduction of postoperative adhesions in the rat uterine horn model with poloxamer 407”, Am. J. Obstet. Gynecol., 162(5):1317. Linsky et al., 1987 “Adhesion reduction in a rabbit uterine horn model using TC-7,” J. Reprod. Med., 32:17, Diamond et al., 1987 “Pathogenesis of adhesions formation/reformation: applications to reproductive surgery,” Microsurgery, 8:103).

For example, formation of post-surgical adhesions involving organs of the peritoneal cavity and the peritoneal wall is undesirable result of abdominal surgery. This occurs frequently and arises from surgical trauma. During the operation, serosanguinous (proteinaceous) exudate is released which tends to collects in the pelvic cavity (Holtz, G., 1984). If the exudate is not absorbed or lysed within a short period it becomes ingrown with fibroblasts, with subsequent collagen deposition occurs leading to adhesions. It is a further embodiment of this invention to administer dendritic macromolecules or combinations of dendritic macromolecules with linear synthetic or natural polymers including peptides for the prevention of adhesions.

H. Drug Delivery

The concept of drug delivery with dendritic macromolecules has been previously explored, (Liu, M. Fréchet, M. J. Pharm. Sci. Technol. Today 1999, 2, 393-401) but the composition of the dendrimers explored was not suited for in vivo application and thus limits their to academic study. In fact these polymers such as PAMAM, have shown increased toxicity with increased generation number. The biodendrimers described in this invention offer many opportunities for designing dendrimers that possess building blocks suitable for in vivo use.

The dendritic polymers of the present invention having pendent heteroatom or functional (e.g., amine, carboxylic acid) groups meet the need for controlling physical properties, derivatizing the polymers with drugs, or altering the biodegradability of the polymers. Therefore, the present invention also includes long and short term implantable medical devices containing the polymers of the present invention. A further embodiment of the present invention, the polymers are combined with a biologically or pharmaceutically active compound (drugs, peptides, nucleic acids, etc) sufficient for effective site-specific or systemic drug delivery (Gutowska et al., J. Biomater. Res., 29, 811-21 (1995) and Hoffman, J. Controlled Release, 6, 297-305 (1987)). The biologically or pharmaceutically active compounds may be physically mixed, embedded in, dispersed in, covalently attached, or adhered to the dendritic macromolecule by hydrogen bonds, salt bridges, ect. Furthermore this invention provides a method for site-specific or systemic drug delivery by implanting in the body of a patient in need thereof an implantable drug delivery device containing a therapeutically effective amount of a biological or pharmaceutical active compound in combination with a polymer of the present invention.

Derivatives of biological or pharmaceutical active compounds, including drugs, can also be attached to the dendritic macromolecule by covalent bonds. This provides for the sustained release of the active compound by means of hydrolysis of the covalent bond between the drug and the polymer backbone as well as by the site of the dug in the dendritic structure (e.g., interior vs. exterior). Many of the pendent groups on the dendritic structure are pH sensitive such as carboxylic acid groups which further controls the pH dependent dissolution rate. Such a dendritic macromolecule may also be used for coating gastrointestinal drug release carriers to protect the entrapped biological or pharmaceutical active compounds such as drugs from degrading in the acidic environment of the stomach. The dendritic polymers of the present invention can be prepared having a relatively high concentration of pendant carboxylic acid groups are stable and insoluble (or slightly soluble) in acidic environments but dissolve/degrade rapidly when exposed to more basic environments. A further embodiment of this invention provides a controlled drug delivery system in which a biologically or pharmaceutically active-agent is physically coated with or covalently attached to a polymer of the invention.

Biodendrimers based on a core unit which is composed of glycerol and lactic acid (glycolic acid, succinic acid for example) represent another class of polymers according to the present invention. The glycerol and lactic acid units in this polymer class are found in vivo and are biocompatible. Thus, one can build a wide range of structures as shown below. After the core is synthesized, polymers such as PEG and PLA can be attached to the core unit to make large starburst or dendritic polymers.

### BRIEF DESCRIPTION OF THE DRAWINGS

FIG. 1 depicts the synthesis route to G0-PGLGA-PHE-OH as descrbed in the Examples below;

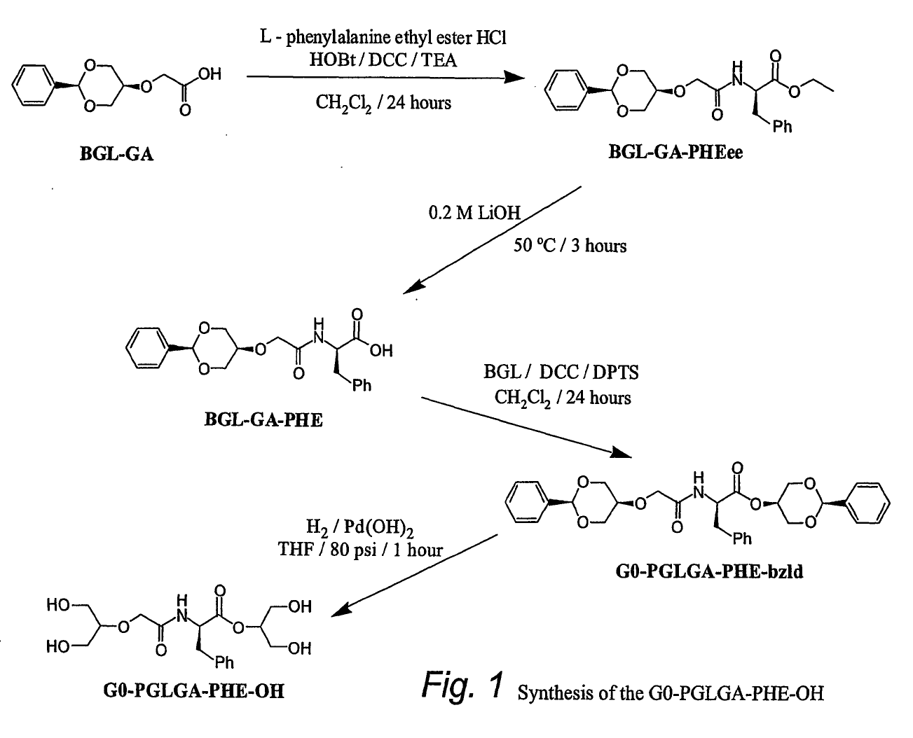

FIG. 2 depicts the synthesis route to G2-PGLGA-PHE-OH as descrbed in the Examples below;

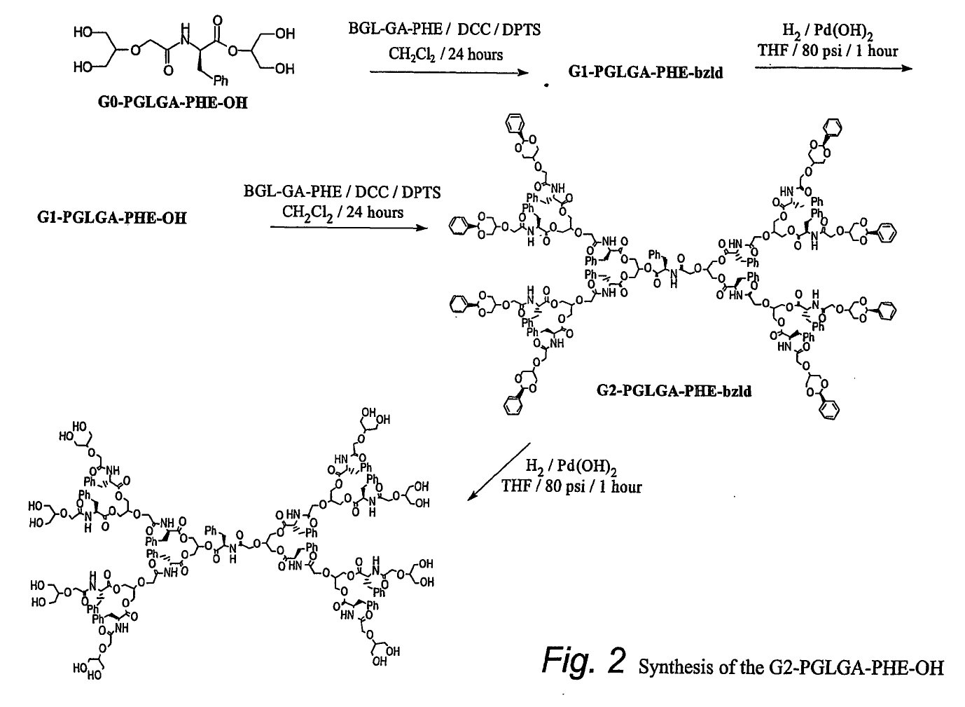

FIG. 3 depicts the synthesis route to G0, G1, G2 and G3 PGLSA-PEG biodendrimers as descrbed in the Examples below;

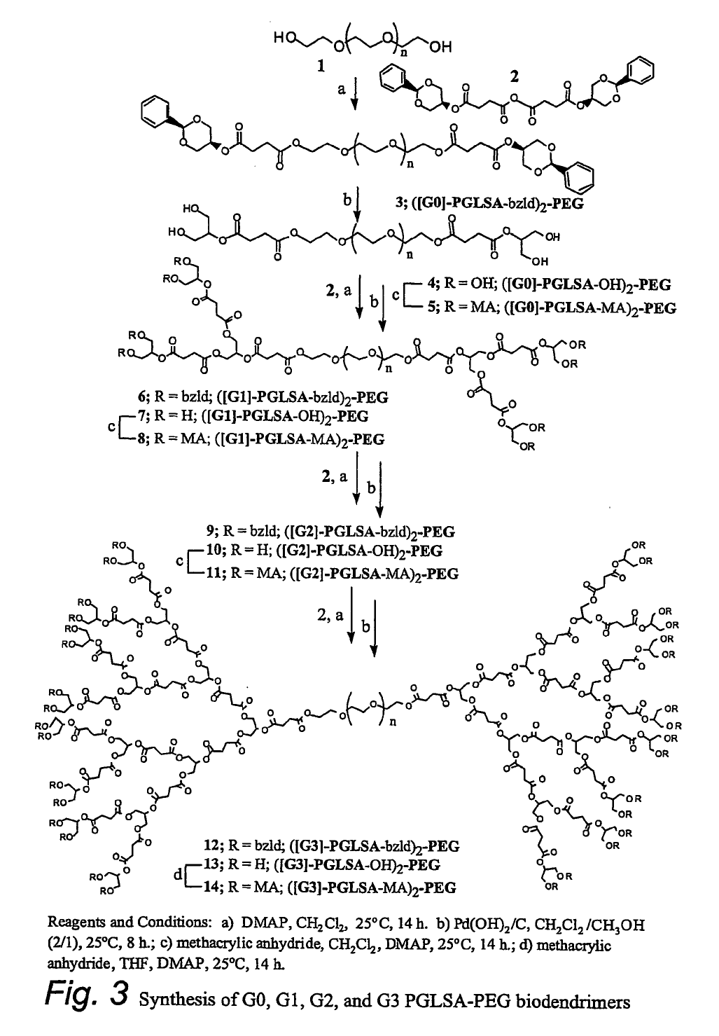

FIG. 4 depicts the synthesis route to G4 PGLSA-PEG biodendrimer as descrbed in the Examples below;

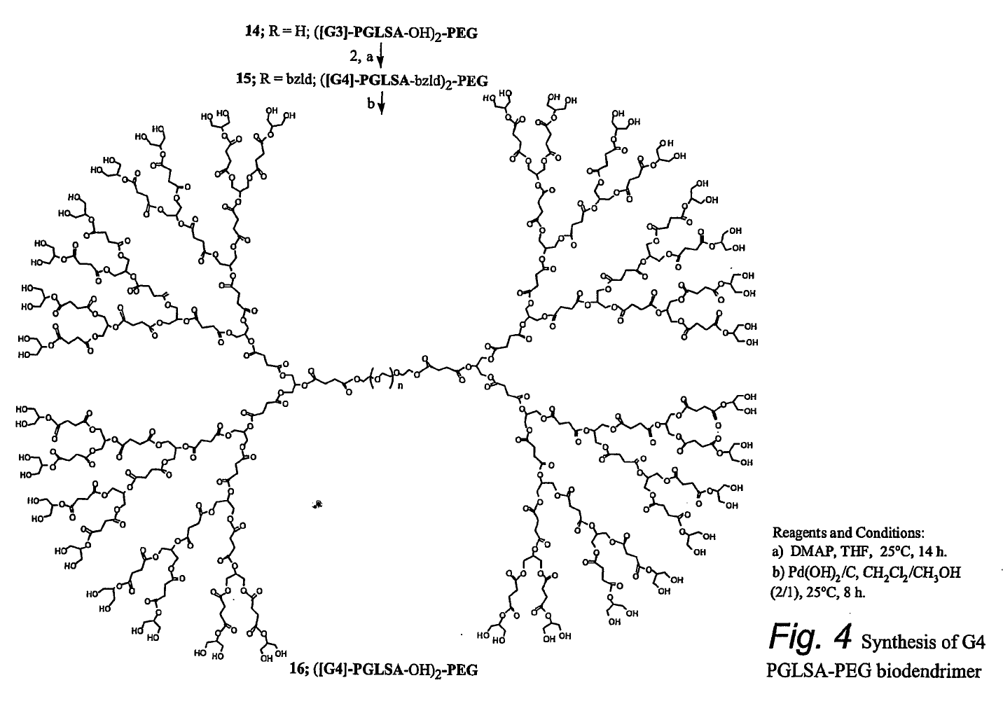

FIG. 5 depicts the synthesis route to G0, G1, G2 and G3 PGLSA biodendrimers as descrbed in the Examples below; and

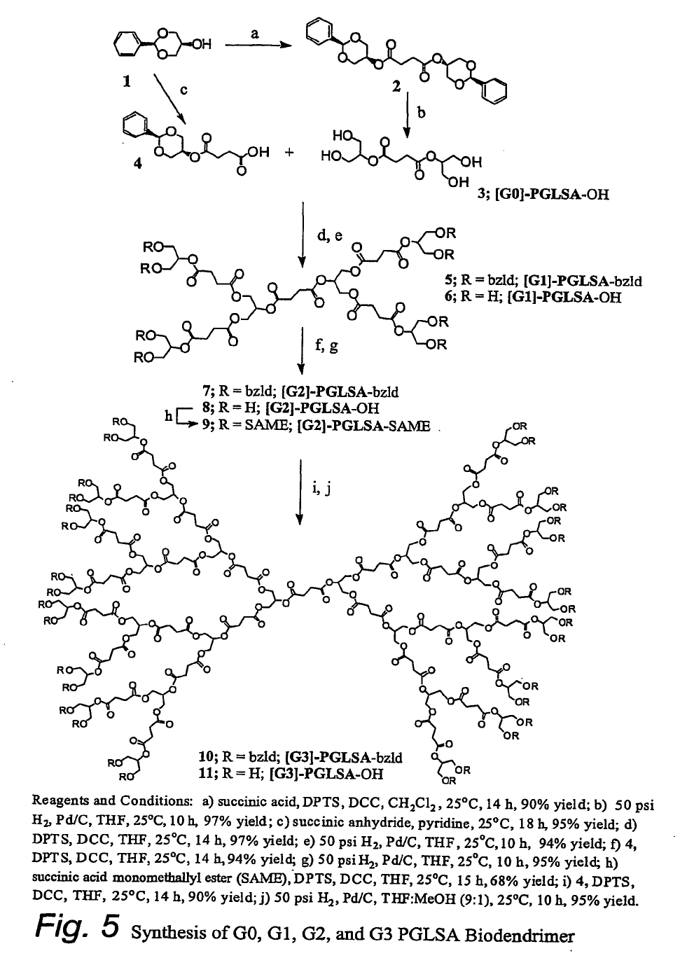

FIG. 6 depicts the synthesis route to G4 PGLSA biodendrimer as descrbed in the Examples below.

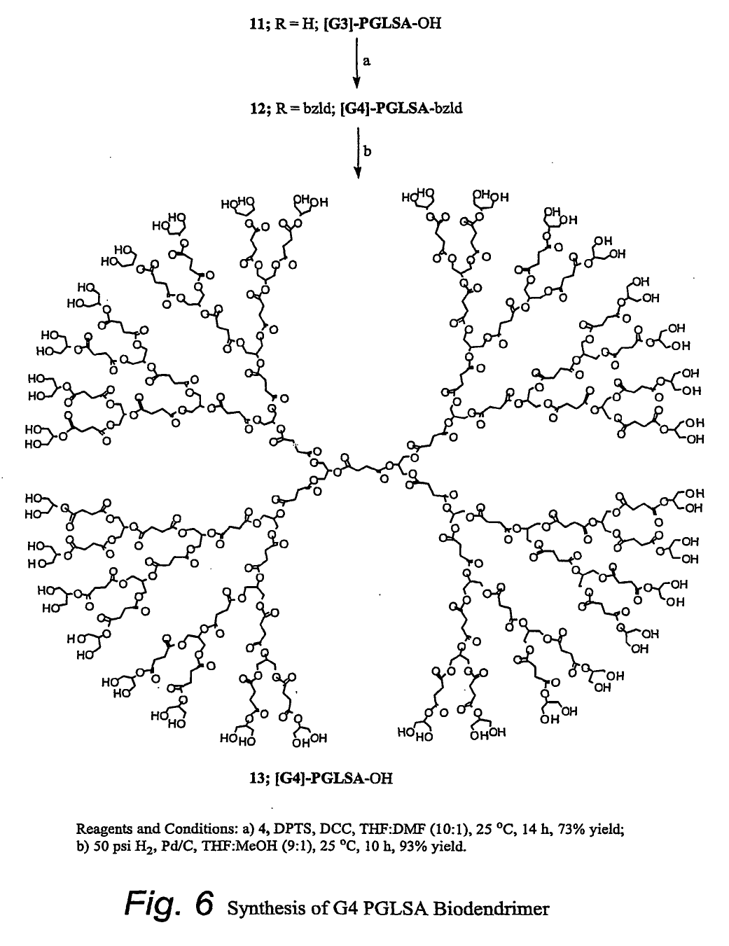

### DETAILED DESCRIPTION OF THE PREFERRED EMBODIMENTS

A more complete understanding of the present invention will be obtained from the following Examples which are intended to be exemplary only and non-limiting to the present invention.

### EXAMPLE 1

Synthesis of 2-[(cis-1,3-benzylidene glycerol)-2-propionic acid]-cis-1,3-O-Benzylidene glycerol (10.9 g, 60.4 mmol) was dissolved in 1,4-dioxane (250 mL) followed by the addition of NaH (7.0 g, 0.30 mol). The reaction mixture was stirred at rt for one hour before cooling to 0° C. 2-Bromopropionic acid (8.64 mL, 96 mmol) was then added over a 15 minute period of time. The reaction mixture was allowed to return to rt and then stirred at 50° C. for 12 hours before it was cooled to 0° C. and quenched with ethanol followed by the addition of water (250 mL). The solution was adjusted to 4.0 pH using 1 N HCl and extracted with CH₂Cl₂ (200 mL). This procedure was repeated once again after re-adjusting the pH to 4.0. The combined organic phase was dried with Na₂SO₄, gravity filtered, and evaporated. The solid was stirred in ethyl ether (50 mL) for 45 minutes and cooled to −25° C. for 3 hours before collecting 11.7 g of the white powder (77.3% yield). ¹H NMR and IR obtained GC-MS 253 m/z (MH⁺) (Theory: 252 m/z (M⁺)) Elemental Analysis C: 61.75%; H 6.37% (Theory: C: 61.90%; H 6.39%).

### EXAMPLE 2

Synthesis of benzylidene protected [G0]-PGLLA—2-[(cis-1,3-benzylidene glycerol)-2-propionic acid] (4.02 g, 15.9 mmol), cis-1,3-O-benzylideneglycerol (2.62 g, 14.5 mmol), and DPTS (1.21 g, 4.10 mmol) were dissolved in CH₂Cl₂ (40 mL). The reaction flask was flushed with nitrogen and then DCC (3.61 g, 17.5 mmol) was added. Stirring at room temperature was continued for 14 hours under a nitrogen atmosphere. Upon reaction completion, the DCC-urea was filtered and washed with a small amount of CH₂Cl₂ (10 mL) and the filtrate was evaporated. The crude product was purified by silica gel chromatography, eluting with 3:97 MeOH:CH₂Cl₂. The product was dissolved in minimal CH₂Cl₂, filtered (to remove any DCU), and precipitated in ethyl ether at −20° C. to remove remaining DCC. Ethyl ether was decanted and the precipitate was exposed to reduced pressure to yield 5.63 g of a white powder (94.0% yield). ¹H NMR obtained GC-MS 415 m/z (MH⁺) (Theory: 414 m/z (M⁺)) Elemental Analysis C: 66.63%; H 6.33% (Theory C: 66.65%; H 6.32%).

### EXAMPLE 3

Synthesis of [G0]-PGLLA—Pd/C (10%) (10% w/w) was added to a solution of benzylidene protected [G0]-PGLLA (5.49 g, 13.2 mmol) in EtOAc/MeOH (3:1, 40 mL). The flask was evacuated and filled with 50 psi of H₂ before shaking for 20 minutes. The catalyst was filtered and washed with EtOAc (10 mL). The filtrate was then evaporated to give 2.94 g of a colorless, viscous oil (94.0% yield). ¹H NMR obtained. (Theory: 238 m/z (M⁺)) Elemental Analysis C: 45.52%; H 7.65% (Theory C: 45.37%; H 7.62%).

### EXAMPLE 4

Synthesis of benzylidene protected [G1]-PGLLA—2-[(cis-1,3-benzylidene glycerol)-2-propionic acid] (4.41 g, 17.50 mmol), [G0]-PGLLA (0.791 g, 3.32 mmol), and DPTS (2:46 g, 8.36 mmol), were dissolved in DMF (80 mL). The reaction flask was flushed with nitrogen and then DCC (5.31 g, 25.74 mmol) was added. The contents were stirred at room temperature for 14 hours under nitrogen atmosphere. The DMF was removed under high vacuum and the remaining residue was dissolved in CH₂Cl₂. The DCC-urea was filtered and washed with a small amount of CH₂Cl₂ (20 mL) and the filtrate was concentrated. The crude product was purified by silica gel chromatography, eluting with 3:97 MeOH:CH₂Cl₂. The product was dissolved in minimal CH₂Cl₂, filtered (to remove any DCU), and precipitated in ethyl ether at −20° C. to remove remaining DCC. Ethyl ether was decanted and the precipitate was exposed to reduced pressure to yield 3.45 g of a white powder (88.3% yield). ¹H NMR obtained FAB MS 1175.6 m/z (MH⁺) (Theory: 1175.2 m/z (M⁺)) Elemental Analysis C: 62.11%; H 6.46% (Theory C: 62.34%; H 6.35%). SEC M_{w}: 1280, Mₙ: 1260, PDI: 1.01.

### EXAMPLE 5

Synthesis of [G1]-PGLLA—Pd/C (10%) (10% w/w) was added to a solution of benzylidene protected [G1]-PGLLA (0.270 g, 0.230 mmol) in THF (15 mL). The flask was evacuated and filled with 50 psi of H₂ before shaking for 15 minutes. The catalyst was filtered and washed with THF (10 mL). The filtrate was then evaporated to give 0.178 g of a colorless, viscous oil (94.0% yield). ¹H NMR obtained FAB MS 823.3 m/z (MH⁺) (Theory: 822.8 m/z (M⁺)) Elemental Analysis C: 47.72%; H 7.41% (Theory C: 48.17%; H 7.11%). SEC M_{w}: 1100, Mₙ: 1090, PDI: 1.01.

### EXAMPLE 6

Synthesis of benzylidene protected [G2]-PGLLA—2-[(cis-1,3-benzylidene glycerol)-2-propionic acid] (8.029 g, 31.83 mmol), DCC (9.140 g, 44.30 mmol), and DPTS (4.629 g, 15.74 mmol) were dissolved in THF (80 mL). The reaction flask was flushed with nitrogen and stirred for 30 minutes before [G1]-PGLLA (0.825 g, 1.00 mmol) was added by dissolving in a minimal amount of THF. The reaction was stirred at room temperature for 14 hours under nitrogen atmosphere. The DCC-urea was filtered and washed with a small amount of THF (20 mL). The THF filtrate was evaporated and the crude product was purified by silica gel chromatography, eluting with 3:97 MeOH:CH₂Cl₂. The product was dissolved in minimal CH₂Cl₂, filtered (to remove any DCU), and precipitated in ethyl ether at −20° C. to remove remaining DCC. Ethyl ether was decanted and the precipitate was exposed to reduced pressure to yield 2.09 g of a white powder (77% yield). ¹H NMR obtained. FAB MS 2697.0 m/z (MH⁺) (Theory: 2696.8 m/z (M⁺)) Elemental Analysis C: 60.86%; H 6.37% (Theory C: 61.02%; H 6.35%). SEC M_{w}: 2350, Mₙ: 2310, PDI: 1.01.

### EXAMPLE 7

Synthesis of [G2]-PGLLA—Pd/C (10%) (10% w/w) was added to a solution of benzylidene protected [G2]-PGLLA (0.095 g, 0.035 mmol) in THF (10 mL). The flask was evacuated and filled with 50 psi of H₂ before shaking for 15 minutes. The catalyst was filtered and washed with THF (10 mL). The filtrate was evaporated to give 0.061 g of a colorless viscous oil (88.0% yield). ¹H NMR obtained MALDI-TOF MS 1991.8 m/z (MH⁺) (Theory: 1991.9 m/z (M⁺)). SEC M_{w}: 2170, Mₙ: 2130, PDI: 1.01.

### EXAMPLE 8

Synthesis of [G2]-PGLLA-Ac—[G2]-PGLLA (0.098 g, 0.049 mmol) was dissolved in 5 mL of pyridine. Acetic anhydride (6.0 mL, 64 mmol) was then added via syringe and the reaction mixture was stirred at 40° C. for 8 hours. Pyridine and acetic anhydride were removed under high vacuum. The product was isolated on a prep TLC eluting with 4:96 MeOH: CH₃Cl. ¹H NMR obtained. FAB MS 2665.0 m/z (MH⁺) (Theory: 2664.5 m/z (M⁺)) Elemental Analysis C: 50.70%; H 6.71% (Theory C: 50.94%; H 6.43%).

### EXAMPLE 9

Synthesis of benzylidene protected [G3]-PGLLA—2-[(cis-1,3-benzylidene glycerol)-2-propionic acid] (0.376 g, 1.49 mmol), DCC (0.463 g, 2.24 mmol), and DPTS (0.200 g, 0.680 mmol) were dissolved in THF (15 mL). The reaction flask was flushed with nitrogen and stirred for 1.5 hours before [G2]-PGLLA (0.070 g, 0.035 mmol) was added by dissolving in a minimal amount of THF. The reaction was stirred at room temperature for 14 hours under nitrogen atmosphere. The DCC-urea was filtered and washed with a small amount of THF (20 mL). The THF filtrate was evaporated and the crude product was purified by silica gel chromatography, eluting with 3:97 MeOH:CH₂Cl₂. The product was dissolved in minimal CH₂Cl₂, filtered (to remove any DCU), and precipitated in ethyl ether at −20° C. to remove remaining DCC. Ethyl ether was decanted and the precipitate was exposed to reduced pressure to yield 0.164 g of a white powder (89.1% yield). ¹H NMR obtained MALDI MS 5743.3 m/z (MH⁺) (Theory: 5739.9 m/z (M⁺)) Elemental Analysis C: 60.32%; H 6.34% (Theory C: 60.47%; H 6.36%). SEC M_{w}: 4370, Mᵣ: 4310, PDI: 1.01.

### EXAMPLE 10

Synthesis of [G3]-PGLLA—Pd/C (10%) (10% w/w) was added to a solution of benzylidene protected [G3]-PGLLA (0.095 g, 0.035 mmol) in THF (15 mL). The flask was evacuated and filled with 50 psi of H₂ before shaking for 15 minutes. The catalyst was filtered and washed with THF (10 mL). The filtrate was evaporated to give 0.128 g of a colorless viscous oil (95.4% yield). ¹H NMR obtained MALDI MS 4332.5 m/z (MH⁺) (Theory: 4330.2 m/z (M⁺)) Elemental Analysis C: 49.56%; H 7.21% (Theory C: 49.09%; H 6.94%). SEC M_{w}: 4110, Mₙ: 4060, PDI: 1.01.

### EXAMPLE 11

Synthesis of [G0]-PGLSA-bzld (2)—Succinic acid (1.57 g, 13.3 mmol), cis-1,3-O-benzylideneglycerol (5.05 g, 28.0 mmol), and DPTS (4.07 g, 13.8 mmol) were dissolved in CH₂Cl₂ (120 mL). The reaction flask was flushed with nitrogen and then DCC (8.19 g, 39.7 mmol) was added. Stirring at room temperature was continued for 14 hours under a nitrogen atmosphere. Upon reaction completion, the DCC-urea was filtered and washed with a small amount of CH₂Cl₂ (20 mL). The crude product was purified by silica gel chromatography, eluting with 3:97 methanol:CH₂Cl₂. The product was dissolved in CH₂Cl₂, filtered (to remove any DCU), and precipitated in ethyl ether at −20° C. to remove remaining DCC. Following vacuum filtration, 5.28 g of a white solid was collected (90% yield). ¹H NMR and IR obtained GC-MS 443 m/z (MH⁺) (Theory: 442 m/z (M⁺)). HR FAB 442.1635 m/z (M⁺) (Theory: 442.1628 m/z (M⁺)). Elemental Analysis C: 65.25%; H 5.85% (Theory C: 65.15%; H 5.92%).

### EXAMPLE 12

Synthesis of [G0]-PGLSA-OH (3)—Pd/C (10% w/w) was added to a solution of benzylidene protected [G0]-PGLSA (2.04 g, 4.61 mmol) in THF (30 mL). The flask for catalytic hydrogenolysis was evacuated and filled with 50 psi of H₂ before shaking for 10 hours. The catalyst was filtered and washed with THF (20 mL). The filtrate was evaporated to give 1.18 g of a clear viscous oil (97% yield). ¹H NMR and IR obtained GC-MS 284 m/z (M+NH₄+) (Theory: 266 m/z (M⁺)). Elemental Analysis C: 44.94%; H 6.87% (Theory C: 45.11%; H 6.81%).

### EXAMPLE 13

Synthesis of 2-(cis-1,3-O-benzylidene glycerol)succinic acid mono ester (4)—cis-1,3-O-Benzylideneglycerol (9.90 g, 54.9 mmol) was dissolved in pyridine (100 mL) followed by the addition of succinic anhydride (8.35 g, 83.4 mmol). The reaction mixture was stirred at room temperature for 18 hours before the pyridine was removed under vacuum at 40° C. The remaining solid was dissolved in CH₂Cl₂ (100 mL) and washed three times with cold 0.2 N HCl (100 mL), or until the aqueous phase remained at pH 1. The organic phase was evaporated and the solid was dissolved in deionized water (300 mL). 1 N NaOH was added until pH 7 was obtained and the product was dissolved in solution. The aqueous phase was extracted with CH₂Cl₂ (200 mL) and then readjusted to pH 4. The aqueous phase was subsequently extracted twice with CH₂Cl₂ (200 mL), dried with Na₂SO₄, filtered, and evaporated. The solid was stirred in ethyl ether (50 mL) and cooled to −25° C. for 3 hours before collecting 14.6 g of a white powder (95% yield). ¹H NMR and IR obtained GC-MS 281 m/z (MH⁺) (Theory: 280 m/z (M⁺)). Elemental Analysis C: 60.07%; H 5.80% (Theory: C: 59.99%; H 5.75%).

### EXAMPLE 14

Synthesis of [G1]-PGLSA-bzld (5)-2-(cis-1,3-O-Benzylidene glycerol)succinic acid mono ester (6.33 g, 22.6 mmol), [G0]-PGLSA (1.07 g, 4.02 mmol), and DPTS (2.51 g, 8.53 mmol) were dissolved in THF (60 mL). The reaction flask was flushed with nitrogen and then DCC (7.04 g, 34.1 mmol) was added. The reaction was stirred at room temperature for 14 hours under nitrogen atmosphere. Upon completion, the DCC-urea was filtered and washed with a small amount of THF (20 mL) and the solvent was evaporated. The crude product was purified by silica gel chromatography, eluting with 3:97 to 5:95 methanol:CH₂Cl₂. The product was dissolved in CH₂Cl₂, filtered (to remove any DCU), and precipitated in ethyl ether at −20° C. to remove remaining DCC. The ethyl ether was decanted and the precipitate was isolated to yield 5.11 g of a white powder (97% yield). ¹H NMR and IR obtained FAB MS 1315.6 m/z (MH⁺) (Theory: 1315.3 m/z (M⁺)). Elemental Analysis C: 60.13%; H 5.82% (Theory C: 60.27%; H 5.67%). SEC M_{w}: 1460, Mₙ: 1450, PDI: 1.01.

### EXAMPLE 15

Synthesis of [G1]-PGLSA-OH (6)—Pd/C (10% w/w) was added to a solution of benzylidene protected [G1]-PGLSA (0.270 g, 0.230 mmol) in THF (20 mL). The flask for catalytic hydrogenolysis was evacuated and filled with 50 psi of H₂ before shaking for 10 hours. The catalyst was filtered and washed with THF (20 mL). The filtrate was evaporated to give 0.178 g of a colorless, viscous oil (94% yield). ¹H NMR and IR obtained FAB MS 963.2 m/z (MH⁺) (Theory: 962.9 m/z (M⁺)). Elemental Analysis C: 47.13%; H 6.11% (Theory C: 47.40%; H 6.07%). SEC M_{w}: 1510, Mₙ: 1500, PDI: 1.01.

### EXAMPLE 16

Synthesis of [G2]-PGLSA-bzld (7)-2-(cis-1,3-O-Benzylidene glycerol)succinic acid mono ester (4.72 g, 16.84 mmol), [G1]-PGLSA (1.34 g, 1.39 mmol), and DPTS (1.77 g, 6.02 mmol) were dissolved in THF (100 mL). The reaction flask was flushed with nitrogen and then DCC (4.62 g, 22.4 mmol) was added. The reaction was stirred at room temperature for 14 hours under nitrogen atmosphere. Upon completion, the DCC-urea was filtered and washed with a small amount of THF (20 mL) and the solvent was evaporated. The crude product was purified by silica gel chromatography, eluting with 3:97 to 5:95 methanol:CH₂Cl₂. The product was dissolved in CH₂Cl₂, filtered (to remove any DCU), and precipitated in ethyl ether at −20° C. to remove remaining DCC. The ethyl ether was decanted and the precipitate was isolated to yield 4.00 g of a white powder (94% yield). ¹H NMR and IR obtained FAB MS 3060.7 m/z (MH⁺) (Theory: 3060.9 m/z (M⁺)). Elemental Analysis C: 59.20%; H 5.64% (Theory C: 58.86%; H 5.60%). SEC M_{w}: 3030, Mₙ: 2990, PDI: 1.01.

### EXAMPLE 17

Synthesis of [G2]-PGLSA-OH (8)—Pd/C (10% w/w) was added to a solution of benzylidene protected [G2]-PGLSA (2.04 g, 0.667 mmol) in THF (20 mL). The flask for catalytic hydrogenolysis was evacuated and filled with 50 psi of H₂ before shaking for 10 hours. The catalyst was filtered and washed with THF (20 mL). The filtrate was evaporated to give 1.49 g of a colorless, viscous oil (95% yield). ¹H NMR and IR obtained MALDI MS 2357.3 m/z (MH⁺) (Theory: 2356.1 m/z (M⁺)). Elemental Analysis C: 48.32%; H 5.97% (Theory C: 47.92%; H 5.90%). SEC M_{w}: 3060, Mᵣ: 3000, PDI: 1.02.

### EXAMPLE 18

Synthesis of succinic acid monomethallyl ester (SAME)-2-Methyl-2-propen-1-ol (4.90 mL, 58.2 mmol) was dissolved in pyridine (20 mL) followed by the addition of succinic anhydride (7.15 g, 71.4 mmol). The reaction mixture was stirred at room temperature for 15 hours before the pyridine was removed under vacuum at 30° C. The remaining liquid was dissolved in CH₂Cl₂ (100 mL) and washed two times with cold 0.2 N HCl (100 mL). The organic phase was dried with Na₂SO₄, gravity filtered, and evaporated to give 9.25 g of a clear liquid (92% yield). ¹H NMR and IR obtained GC-MS 173 m/z (MH⁺) (Theory: 172 m/z (M⁺)). Elemental Analysis C: 55.51%; H 7.09% (Theory: C: 55.81%; H 7.02%).

### EXAMPLE 19

Synthesis of [G2]-PGLSA-SAME (9)—Succinic acid monomethallyl ester (0.826 g, 4.80 mmol), [G2]-PGLSA (0.401 g, 0.170 mmol), and DPTS (0.712 g, 2.42 mmol) were dissolved in THF (50 mL). The reaction flask was flushed with nitrogen and then DCC (1.52 g, 7.37 mmol) was added. Stirring at room temperature was continued for 14 hours under nitrogen atmosphere. Upon completion, the DCC-urea was filtered and washed with a small amount of CH₂Cl₂ (20 mL) and the solvent was evaporated. The crude product was purified by silica gel chromatography, eluting with 3:97 to 5:95 methanol:CH₂Cl₂. The product was dissolved in CH₂Cl₂, filtered (to remove any DCU), and precipitated in ethyl ether at −20° C. to remove remaining DCC. The ethyl ether was decanted and the precipitate was isolated to yield 0.558 g of a clear colorless oil (68.2% yield). ¹H NMR and IR obtained MALDI MS 4840.9 m/z (MH⁺) (Theory: 4838.7 m/z (M⁺)). Elemental Analysis C: 55.37%; H 6.22% (Theory C: 55.35%; H 6.29%). SEC M_{w}: 5310, Mₙ: 5230, PDI: 1.02.

### EXAMPLE 20

Synthesis of [G3]-PGLSA-bzld (10)-2-(cis-1,3-O-Benzylidene glycerol)succinic acid mono ester (2.77 g, 9.89 mmol), [G2]-PGLSA (1.00 g, 0.425 mmol), and DPTS (1.30 g, 4.42 mmol) were dissolved in THF (40 mL). The reaction flask was flushed with nitrogen and then DCC (2.67 g, 12.9 mmol) was added. The reaction was stirred at room temperature for 14 hours under nitrogen atmosphere. Upon completion, the DCC-urea was filtered and washed with a small amount of THF (20 mL) and the solvent was evaporated. The crude product was purified by silica gel chromatography, eluting with 3:97 to 5:95 methanol:CH₂Cl₂. The product was dissolved in CH₂Cl₂, filtered (to remove any DCU), and precipitated in ethyl ether at −20° C. to remove remaining DCC. The ethyl ether was decanted and the precipitate was isolated to yield 3.51 g of a white powder (90% yield). ¹H NMR and IR obtained MALDI MS 6553.4 m/z (MH⁺) (Theory: 6552.2 m/z (M⁺)). Elemental Analysis C: 58.50%; H 5.66% (Theory C: 58.29%; H 5.57%). SEC M_{w}: 5550, Mₙ: 5480, PDI: 1.01.

### EXAMPLE 21

Synthesis of [G3]-PGLSA-OH (11)-Pd/C (10% w/w) was added to a solution of benzylidene protected [G3]-PGLSA (1.23 g, 0.188 mmol) in 9:1 THF/MeOH (20 mL). The flask for catalytic hydrogenolysis was evacuated and filled with 50 psi of H₂ before shaking for 10 hours. The catalyst was filtered and washed with 9:1 THF/MeOH (20 mL). The filtrate was evaporated to give 0.923 g of a colorless, viscous oil (95% yield). ¹H NMR and IR obtained MALDI MS 5144.8 m/z (MH⁺) (Theory: 5142.5 m/z (M⁺)). Elemental Analysis C: 48.07%; H 5.84% (Theory C: 48.11%; H 5.84%). SEC M_{w}: 5440, Mₙ: 5370, PDI: 1.01.

### EXAMPLE 22

Synthesis of [G4]-PGLSA-bzld (12)—2-(cis-1,3-O-Benzylidene glycerol)succinic acid mono ester (2.43 g, 8.67 mmol), [G3]-PGLSA (0.787 g, 0.153 mmol), and DPTS (1.30 g, 4.42 mmol) were dissolved in 10:1 THFIDMF (40 mL). The reaction flask was flushed with nitrogen and then DCC (2.63 g, 12.7 mmol) was added. The reaction was stirred at room temperature for 14 hours under nitrogen atmosphere. Upon completion, solvents were removed under vacuum and the remaining solids were redissolved CH₂Cl₂. The DCC-urea was filtered and washed with a small amount of CH₂Cl₂ (20 mL) and the solvent was evaporated. The crude product was purified by silica gel chromatography, eluting with 3:97 to 5:95 methanol:CH₂Cl₂. The product was dissolved in CH₂Cl₂, filtered (to remove any DCU), and precipitated in ethyl ether at −20° C. to remove remaining DCC. The ethyl ether was decanted and the precipitate was exposed to reduced pressure to yield 1.50 g of a white powder (73% yield). ¹H NMR and IR obtained MALDI MS 13536.8 m/z (MH⁺) (Theory: 13534.7 m/z (M⁺)). Elemental Analysis C: 58.20%; H 5.56% (Theory C: 58.04%; H 5.56%). SEC M_{w}: 9000, Mₙ: 8900, PDI: 1.01.

### EXAMPLE 23

Synthesis of [G4]-PGLSA-OH (13)—Pd/C (10% w/w) was added to a solution of benzylidene protected [G4]-PGLSA (0.477 g, 0.0352 mmol) in 9:1 THF/MeOH (20 mL). The flask for catalytic hydrogenolysis was evacuated and filled with 50 psi of H₂ before shaking for 10 hours. The catalyst was filtered and washed with 9:1 THF/IMeOH (20 mL). The filtrate was evaporated to give 0.351 g of a colorless, viscous oil (93% yield). ¹H NMR and IR obtained MALDI MS 10715.6 m/z (MH⁺) (Theory: 10715.3 m/z (M⁺)). Elemental Analysis C: 48.50%; H 5.83% (Theory C: 48.20%; H 5.81%). SEC M_{w}: 8800, Mₙ: 8720, PDI: 1.01.

### EXAMPLE 24

Polymerization of [G2]-PGLSA-SAME—Gels were prepared by dissolving [G2]-PGLSA-SAME and DMPA (0.1% w/w) in CH₂Cl₂ to make 10% w/w solutions. One drop of solution was applied from a pipet tip onto a fresh mica surface and immediately exposed to UV light from a UVP BLAK-RAY long wave ultraviolet lamp for 15 minutes. The surface was washed with 1.0 mL of hexane and allowed to dry overnight.

### EXAMPLE 25

Photomask polymerization of [G2]-PGLSA-SAME—Gels were prepared by dissolving [G2]-PGLSA-SAME, DMPA, and VP (1,000:10:1 respectively) in CH₂Cl₂ and the solution was concentrated. Next, a small amount of the polymer (with initiator and accelerator) was dissolved in a minimal amount of CH₂Cl₂ to allow spin coating of a glass cover slip. A photo mask was placed on top of this cover slip and exposed to UV light from a UVP BLAK-RAY long wave ultraviolet lamp for 15 minutes. The surface was washed with 1.0 mL of hexane and allowed to air-dry overnight.

### EXAMPLE 26

Synthesis of 2-(cis-1,3-O-benzylidene glycerol)succinic acid mono ester anhydride (2)—2-(cis-1,3-O-Benzylidene glycerol)succinic acid mono ester (50.00 g, 178.4 mmol)) and DCC (22.09 g, 107.0 mmol) were dissolved in DCM (300 mL) and stirred for 14 hours. The DCU precipitate was collected by filtration and washed with DCM (50 mL). The organic phase was directly added to 900 mL of hexanes. The hexanes and precipitate were cooled to −20° C. for 3 hours before 46.11 g of precipitate was collected after filtration (95% yield). ¹H NMR and IR obtained FAB-MS 543.2 m/z (MH⁺) (Theory: 542.53 m/z (M⁺)). Elemental Analysis C: 61.83%; H 5.70% (Theory: C: 61.99%; H 5.57%).

### EXAMPLE 27

Synthesis of ([G0]-PGLSA-bzld)₂-PEG (3)—PEG, Mₙ₌3400, (5.00 g, 1.49 mmol), which was dried under vacuum at 120° C. for three hours and 2-(cis-1,3-O-benzylidene glycerol)succinic acid mono ester anhydride (4.10 g, 7.56 mmol) were dissolved in DCM (25 mL) and stirred under nitrogen. DMAP (67.0 mg, 0.548 mmol) was added and stirring was continued for 14 hours. Any remaining anhydride was quenched by the addition of n-propanol (1.0 mL, 11 mmol), which was allowed to stir for another 5 hours. The reaction was diluted with DCM (25 mL) and washed with 0.1 N HCl (50 mL), saturated sodium bicarbonate (50 mL 3×), and brine (50 mL). The organic phase was dried with Na₂SO₄ and filtered before the PEG-based dendrimer was precipitated in cold (−20° C.) ethyl ether (500 mL) and collected to yield 5.22 g of a white solid (91% yield). ¹H NMR and IR obtained MALDI MS M_{w}: 3960, Mₙ: 3875, PDI: 1.02. SEC M_{w}: 3880, Mₙ: 3750, PDI: 1.04. Tₘ=44.7.

### EXAMPLE 28

Synthesis of ([G0]-PGLSA-OH)₂-PEG (4)—Pd(OH)₂/C (10% w/w) was added to a solution of ([G0]-PGLSA-bzld)₂-PEG (4.98 g, 1.28 mmol) in 30 mL of 2:1 DCM/methanol. The apparatus for catalytic hydrogenolysis was evacuated and filled with 60 psi of H₂ before shaking for 8 hours. The catalyst was filtered off and washed with DCM (20 mL). The filtrate was concentrated and the PEG-based dendrimer was precipitated in cold (−20° C.) ethyl ether (500 mL) to give 4.63 g of a white solid (97% yield). ¹H NMR and IR obtained MALDI MS M_{w}: 3769, Mₙ: 3696, PDI: 1.02. SEC M_{w}: 3640, Mₙ: 3500, PDI: 1.04. Tₘ=46.6.

### EXAMPLE 29

Synthesis of ([G0]-PGLSA-MA)₂-PEG (5)—([G0]-PGLSA-OH)₂-PEG (0.502 g, 0.135 mmol) was dissolved in DCM (15 mL) and stirred under nitrogen before methacrylic anhydride (0.35 mL, 2.35 mmol) was added by syringe. DMAP (52.0 mg, 0.426 mmol) was added and stirring was continued for 14 hours. Any remaining anhydride was quenched by the addition of methanol (0.1 mL, 3.95 mmol), which was allowed to stir for another 5 hours. The reaction was diluted with DCM (35 mL) and washed with 0.1 N HCl (50 mL) and brine (50 mL). The organic phase was dried with Na₂SO₄ and filtered before the PEG-based dendrimer was precipitated in cold (−20° C.) ethyl ether (300 mL) and collected to yield 0.497 g of a white-solid (93% yield). ¹H NMR and IR obtained MALDI MS M_{w}: 3996, Mₙ: 3914, PDI: 1.02. SEC M_{w}: 3680, Mₙ: 3520, PDI: 1.04. Tₘ=46.3.

### EXAMPLE 30

Synthesis of ([G1]-PGLSA-bzld)₂-PEG (6)—([G0]-PGLSA-OH)₂-PEG (4.33 g, 1.17 mmol), and 2-(cis-1,3-O-benzylidene glycerol)succinic acid mono ester anhydride (9.99 g, 18.4 mmol) were dissolved in DCM (30 mL) and stirred under nitrogen. DMAP (63.7 mg, 0.480 mmol) was added and stirring was continued for 14 hours. Any remaining anhydride was quenched by the addition of n-propanol (2.0 mL, 22 mmol), which was allowed to stir for another 5 hours. The reaction was diluted with DCM (45 mL) and washed with 0.1 N HCl (75 mL), saturated sodium bicarbonate (75 mL 3×), and brine (75 mL). The organic phase was dried with Na₂SO₄ and filtered before the PEG-based dendrimer was precipitated in cold (−20° C.) ethyl ether (500 mL) and collected to yield 5.15 g of a white solid (93% yield). ¹H NMR and IR obtained MALDI MS M_{w}: 4844, Mₙ: 4749, PDI: 1.02. SEC M_{w}: 3950, Mₙ: 3790, PDI: 1.04. Tₘ=38.8.

### EXAMPLE 31

Synthesis of ([G1]-PGLSA-OH)₂-PEG (7)—Pd(OH)₂/C (10% w/w) was added to a solution of ([G1]-PGLSA-bzld)₂-PEG (4.64 g, 0.974 mmol) in 20 mL of 2:1 DCM/methanol. The apparatus for catalytic hydrogenolysis was evacuated and filled with 60 psi of H₂ before shaking for 8 hours. The catalyst was filtered off and washed with DCM (20 mL). The filtrate was concentrated and the PEG-based dendrimer was precipitated in cold (−20° C.) ethyl ether (500 mL) to give 4.00 g of a white solid (93% yield). ¹H NMR and IR obtained MALDI MS M_{w}: 4487, Mₙ: 4394, PDI: 1.02. SEC M_{w}: 4590, Mₙ: 4440, PDI: 1.03. Tₘ=41.9.

### EXAMPLE 32

Synthesis of ([G1]-PGLSA-MA)₂-PEG (8)—([G1]-PGLSA-OH)₂-PEG (0.500 g, 0.113 mmol) was dissolved in DCM (15 mL) and stirred under nitrogen before methacrylic anhydride (0.56 mL, 3.76 mmol) was added by syringe. DMAP (86.0 mg, 0.704 mmol) was added and stirring was continued for 14 hours. Any remaining anhydride was quenched by the addition of methanol (0.1 mL, 3.95 mmol), which was allowed to stir for another 5 hours. The reaction was diluted with DCM (35 mL) and washed with 0.1 N HCl (50 mL) and brine (50 mL). The organic phase was dried with Na₂SO₄ and filtered before the PEG-based dendrimer was precipitated in cold (−20° C.) ethyl ether (300 mL) and collected to yield 0.519 g of a white solid (93% yield). ¹H NMR and IR obtained MALDI MS M_{w}: 5012, Mₙ: 4897, PDI: 1.02. SEC M_{w}: 3910, Mₙ: 3740, PDI: 1.04. Tₘ=40.8.

### EXAMPLE 33

Synthesis of ([G2]-PGLSA-bzld)₂-PEG (9)—([G1]-PGLSA-OH)₂-PEG (3.25 g, 0.737 mmol), and 2-(cis-1,3-O-benzylidene glycerol)succinic acid mono ester anhydride (12.68 g, 23.37 mmol) were dissolved in DCM (50 mL) and stirred under nitrogen. DMAP (0.588 g, 4.81 mmol) was added and stirring was continued for 14 hours. Any remaining anhydride was quenched by the addition of n-propanol (2.5 mL, 28 mmol), which was allowed to stir for another 5 hours. The reaction was diluted with DCM (50 mL) and washed with 0.1 N HCl (100 mL), saturated sodium bicarbonate (100 mL 3×), and brine (100 mL). The organic phase was dried with Na₂SO₄, filtered, and concentrated before the PEG-based dendrimer was precipitated in cold (−20° C.) ethyl ether (400 mL) and collected to yield 4.57 g of a white solid (91% yield). ¹H NMR and IR obtained MALDI MS M_{w}: 6642, Mₙ: 6492, PDI: 1.02. SEC M_{w}: 4860, Mₙ: 4680, PDI: 1.04. Tₘ=31.4.

### EXAMPLE 34

Synthesis of ([G2]-PGLSA-OH)₂-PEG (10)—Pd(OH)₂/C (10% w/w) was added to a solution of ([G2]-PGLSA-bzld)₂-PEG—(3.26 g, 0.500 mmol) in 25 mL of 2:1 DCM/methanol. The apparatus for catalytic hydrogenolysis was evacuated and filled with 60 psi of H₂ before shaking for 8 hours. The catalyst was filtered off and washed with DCM (20 mL). The PEG-based dendrimer was isolated after evaporation of solvents to give 2.86 g of a white solid (98% yield).

¹H NMR and IR obtained MALDI MS M_{w}: 5910, Mₙ: 5788, PDI: 1.02. SEC M_{w}: 5340, Mₙ: 5210, PDI: 1.03. Tₘ=36.5.

### EXAMPLE 35

Synthesis of ([G2]-PGLSA-MA)₂-PEG (11)—([G2]-PGLSA-OH)₂-PEG (0.501 g, 0.0863 mmol) was dissolved in DCM (15 mL) and stirred under nitrogen before methacrylic anhydride (0.50 mL, 3.36 mmol) was added by syringe. DMAP (72.1 mg, 0.990 mmol) was added and stirring was continued for 14 hours. Any remaining anhydride was quenched by the addition of methanol (0.1 mL, 3.95 mmol), which was allowed to stir for another 5 hours. The reaction was diluted with DCM (35 mL) and washed with 0.1 N HCl (50 mL) and brine (50 mL). The organic phase was dried with Na₂SO₄ and filtered before the PEG-based dendrimer was precipitated in cold (−20° C.) ethyl ether (300 mL) and collected to yield 0.534 g of a white solid (90% yield). ¹H NMR and IR obtained MALDI MS M_{w}: 6956, Mₙ: 6792, PDI: 1.02. SEC M_{w}: 4580, Mₙ: 4390, PDI: 1.04. Tₘ=27.0.

### EXAMPLE 36

Synthesis of ([G3]-PGLSA-bzld)₂-PEG (12)—([G2]-PGLSA-OH)₂-PEG (2.13 g, 0.367 mmol), and 2-(cis-1,3-O-benzylidene glycerol)succinic acid mono ester anhydride (12.71 g, 23.43 mmol) were dissolved in DCM (45 mL) and stirred under nitrogen. DMAP (0.608 g, 4.98 mmol) was added and stirring was continued for 14 hours. Any remaining anhydride was quenched by the addition of n-propanol (2.0 mL, 22 mmol), which was allowed to stir for another 5 hours. The reaction was diluted with DCM (55 mL) and washed with 0.1 N HCl (100 mL), saturated sodium bicarbonate (100 mL 3×), and brine (100 mL). The organic phase was dried with Na₂SO₄, filtered, and concentrated before the PEG-based dendrimer was precipitated in cold (−20° C.) ethyl ether (400 mL) overnight and collected to yield 3.35 g of a white solid (92% yield). ¹H NMR and IR obtained MALDI MS M_{w}: 10215, Mₙ: 9985, PDI: 1.02. SEC M_{w}: 7020, Mₙ: 6900, PDI: 1.02. T_{g}=−13.6.

### EXAMPLE 37

Synthesis of ([G3]-PGLSA-OH)₂-PEG (13)—Pd(OH)₂/C (10% w/w) was added to a solution of ([G3]-PGLSA-bzld)₂-PEG (2.88 g, 0.288 mmol) in 30 mL of 2:1 DCM/methanol. The apparatus for catalytic hydrogenolysis was evacuated and filled with 60 psi of H₂ before shaking for 8 hours. The catalyst was filtered off and washed with DCM (20 mL). The PEG-based dendrimer was isolated after evaporation of solvents to give 2.86 g of a white solid (98% yield). ¹H NMR and IR obtained MALDI MS Mₙ: 8765, Mₙ: 8575, PDI: 1.02. SEC M_{w}: 8090, Mₙ: 7820, PDI: 1.03. T_{g}=−38.2.

### EXAMPLE 38

Synthesis of ([G3]-PGLSA-MA)₂-PEG (14)—([G3]-PGLSA-OH)₂-PEG (0.223 g, 0.0260 mmol) was dissolved in THF (15 mL) and stirred under nitrogen before methacrylic anhydride (1.10 mL, 7.38 mmol) was added by syringe. DMAP (90.0 mg, 0.737 mmol) was added and stirring was continued for 14 hours. Any remaining anhydride was quenched by the addition of methanol (0.2 mL, 7.89 mmol), which was allowed to stir for another 5 hours. The reaction was diluted with DCM (35 mL) and washed with 0.1 N HCl (50 mL) and brine (50 mL). The organic phase was dried with Na₂SO₄ and filtered before the PEG-based dendrimer was precipitated in cold (−20° C.) ethyl ether (300 mL) and collected to yield 0.248 g of a white solid (89% yield). ¹H NMR and IR obtained MALDI MS M_{w}: 10722, Mₙ: 10498, PDI: 1.02. SEC M_{w}: 7000, Mₙ: 6820, PDI: 1.03. T_{g}=−37.9.

### EXAMPLE 39

Synthesis of ([G4]-PGLSA-bzld)₂-PEG (15)—([G3]-PGLSA-OH)₂-PEG (1.82 g, 0.212 mmol), and 2-(cis-1,3-O-benzylidene glycerol)succinic acid mono ester anhydride (15.93 g, 29.36 mmol) were dissolved in THF (50 mL) and stirred under nitrogen. DMAP (0.537 g, 4.40 mmol) was added and stirring was continued for 14 hours. Any remaining anhydride was quenched by the addition of n-propanol (2.5 mL, 28 mmol), which was allowed to stir for another 5 hours. The reaction was diluted with DCM (50 mL) and washed with 0.1 N HCl (100 mL), saturated sodium bicarbonate (100 mL 3×), and brine (100 mL). The organic phase was dried with Na₂SO₄, filtered, and concentrated before the PEG-based dendrimer was precipitated in ethyl ether (400 mL) and collected to yield 3.11 g of a white solid (87% yield). ¹H NMR and IR obtained MALDI MS M_{w}: 17289, Mₙ: 16968, PDI: 1.02. SEC M_{w}: 8110, Mₙ: 7950, PDI: 1.02. T_{g}=5.3.

### EXAMPLE 40

Synthesis of ([G4]-PGLSA-OH)₂-PEG (16)—Pd(OH)₂/C (10% w/w) was added to a solution of ([G4]-PGLSA-bzld)₂-PEG (2.88 g, 0.170 mmol) in 30 mL of 2:1 DCM/methanol. The apparatus for catalytic hydrogenolysis was evacuated and filled with 60 psi of H₂ before shaking for 8 hours. The catalyst was filtered off and washed with DCM (20 mL). The PEG-based dendrimer was isolated after evaporation of solvents to give 2.86 g of a white solid (98% yield).

¹H NMR and IR obtained MALDI MS M_{w}: 14402, Mₙ: 14146, PDI: 1.02. SEC M_{w}: 9130, Mₙ: 8980, PDI: 1.02. T_{g}=−18.0.

### EXAMPLE 41

General Preparation of ([Gn]-PGLSA-MA)₂-PEG dendrimers for use as a corneal tissue adhesive—As an example, ([G1]-PGLSA-MA)₂-PEG (0.100 g, 0.202 mmol) was dissolved in ethanol (polymer:solvent ratio of 2.5:1 (w/w)). Once the eyes were prepared, 5 μL of a photoinitiating system containing 5 μL of 0.5% EY in DI water, 50 μL of 5M triethanolamine, and 1 μL of VP was added and mixed thoroughly.

### EXAMPLE 42

General Procedure for the Eye Surgeries. An enucleated human eye (NC Eye Bank) was placed under a surgical microscope with the cornea facing upwards. The corneal epithelium was scraped with a 4.1 mm keratome blade, and then a 2.75 mm keratome blade was used to incise the central cornea. Next the keratome blade was used to form the 4.1 mm linear laceration. The wound was closed with either 3 interrupted 10-0 nylon sutures or the photocrosslinkable biodendritic copolymer. The polymer containing the photoinitiating system was then applied to the wound in the following manner. First, 10 μL of 5, 8, 11, or 14 was collected in a tuberculin syringe using a 23 gauge needle. Next the photocrosslinkable dendrimer was applied using the same syringe in a thin band along the length of the linear incision (about 1 mm width and 5 mm length). An argon-ion laser (Coherent; with the fiber optic attachment installed) irradiated the copolymer, at a distance of 0.5 cm from the eye while moving the laser beam along the applied copolymer to initiate photopolymerization (200 mW, 1 second pulse exposures, 50 total pulses). Next, a 25 gauge butterfly needle connected to a syringe pump (kdScientific, Model 100 series) was inserted into the scleral wall adjacent to an ocular muscle. In order to measure the wound leaking pressures, the eye was connected to a cardiac transducer via a 20 gauge needle which was inserted 1 cm through the optic nerve. The needle was held in place with surgical tape. The pressure was then recorded. The syringe pump dispensed buffered saline solution (at a rate of 15-20 mUhr) into the eye while the pressure was simultaneously read on the cardiac transducer. The syringe pump rate was maintained to achieve a continuous 1 mm Hg increase in pressure. The leak pressure was recorded as the pressure at which fluid was observed to leak from the eye under the surgical microscope.

An enucleated eye with the cornea facing upwards was held under a surgical microscope and a 4.1 mm laceration was made with a keratome blade. This wound was then closed using either three interrupted 10-0 nylon sutures in a standard 3-1-1 suturing configuration or the photocrosslinkable biodendritic copolymer (see Scheme 1). Specifically, 10 μL of copolymer 5, 8, 11, or 14 was applied to the laceration and argon ion laser irradiation produced the dendritic gel sealing the wound (200 mW, 1 sec exposures; 50 sec total irradiation time; the polymer solution contained ethyl eosin in 1-vinyl pyrrolidinone and TEA as photoinitiator and co-catalyst). Next, saline was injected in the anterior chamber via a syringe inserted through the scleral wall adjacent to an ocular muscle until the repaired laceration leaked. A cardiac transducer probe inserted approximately 1 cm through the optic nerve monitored the leaking pressure for both the nylon suture (N=6) and biodendrimer sealant (N=3; for each copolymer tested) treated eyes. For reference, normal intraocular pressure in a human eye is between 18 and 20 mm Hg. The mean leaking pressures (LP) for the sutured treated eyes was 90±18 mm Hg. The LP for the eyes sealed with copolymer 8 was 171±44 mm Hg (range 142 to 222 mm Hg). Copolymer 5 did not seal the wound and leaked before measurements could be obtained. Copolymer 11 polymerized too quickly under the operating microscope to be delivered to the wound in a controlled fashion (LP<15 mm Hg). Copolymer 14 was insoluble in water and only slightly soluble in alcohols, and when applied to the laceration did not seal the wound.

### EXAMPLE 43

Synthesis of 2-[(cis-1,3-benzylidene glycerol)-2-acetate glycine ethyl ester]. 2-[(cis-1,3-benzylidene glycerol)-2-acetic acid] (4.02 g, 16.9 mmol), glycine ethyl ester (3.53 g, 25.3 mmol), and DCC (5.22 g, 25.3 mmol) were dissolved in CH₂Cl₂ (40 mL). Stirring at room temperature was continued for 14 hours under a nitrogen atmosphere with TEA. Upon reaction completion, the DCC-urea was filtered and washed with a small amount of CH₂Cl₂ (10 mL) and the filtrate was evaporated. The crude product was purified by silica gel chromatography, eluting with MeOH:CH₂Cl₂. The product was dissolved in minimal CH₂Cl₂, filtered (to remove any DCU), and precipitated in ethyl ether at −20° C. to remove remaining DCC. Ethyl ether was decanted and the precipitate was exposed to reduced pressure to yield 2.07 g of a white powder (38.0% yield). ¹H NMR and IR obtained GC-MS 324 m/z (MH⁺) (Theory: 323 m/z (M⁺)) FAB-MS.

### EXAMPLE 44

Synthesis of 2-[(cis-1,3-benzylidene glycerol)-2-acetate glycine] 2-[(cis-1,3-benzylidene glycerol)-2-acetate glycine ethyl ester was dissloved in DMF and NaOH was added. ¹H NMR obtained FAB-MS.

### EXAMPLE 45

Synthesis of benzylidene protected [G0]-PGLGA-GLY 2-[(cis-1,3-benzylidene glycerol)-2-acetate glycine] (4.02 g, 15.9 mmol), cis-1,3-O-benzylideneglycerol (2.62 g, 14.5 mmol), and DPTS (1.21 g, 4.10 mmol) were dissolved in CH₂Cl₂ (40 mL). The reaction flask was flushed with nitrogen and then DCC (3.61 g, 17.5 mmol) was added. Stirring at room temperature was continued for 14 hours under a nitrogen atmosphere. Upon reaction completion, the DCC-urea was filtered and washed with a small amount of CH₂Cl₂ (10 mL) and the filtrate was evaporated. The crude product was purified by silica gel chromatography, eluting with 3:97 MeOH:CH₂Cl₂. The product was dissolved in minimal CH₂Cl₂, filtered (to remove any DCU), and precipitated in ethyl ether at −20° C. to remove remaining DCC. Ethyl ether was decanted and the precipitate was exposed to reduced pressure to yield 5.63 g of a white powder (94.0% yield). ¹H NMR obtained GC-MS 415 m/z (MH⁺) (Theory: 414 m/z (M⁺)) Elemental Analysis C: 66.63%; H 6.33% (Theory C: 66.65%; H 6.32%).

### EXAMPLE 46

Synthesis of [G0]-PGLGA-GLY—Pd/C (10%) (10% w/w) was added to a solution of benzylidene protected [G0]-PGLGA-GLY (5.49 g, 13.2 mmol) in EtOAc/MeOH (3:1, 40 mL). The flask was evacuated and filled with 50 psi of H₂ before shaking for 20 minutes. The catalyst was filtered and washed with EtOAc (10 mL). The filtrate was then evaporated to give 2.94 g of a colorless, viscous oil (94.0% yield). ¹H NMR and IR obtained. (Theory: 238 m/z (M⁺)) Elemental Analysis C: 45.52%; H 7.65% (Theory C: 45.37%; H 7.62%).

### EXAMPLE 47

Hyperbranched Biodendrimer: Stirring a solution of the NHS protected ester of the 2-O-(succinic acid) glycerol derivative in the presence of TEA yielded a hyperbranched polymer. NMR obtained. With 1 equivalent of the tertra-functional core with 60 equivalents of the NHS ester affords a biodendritic hyperbranched polymer of weight approximately 10 kD.

### EXAMPLE 48

Polymerization of [G2]-PGLSA-MA—Gels were prepared by dissolving [G2]-PGLSA-MA and DMPA (0.1% w/w) in CH₂Cl₂ to make and 10% w/w solutions. One drop of solution was applied from a pipet tip onto a fresh mica surface and immediately exposed to UV light from a UVP BLAK-RAY long wave ultraviolet lamp for 15 minutes. The surface was washed with 1.0 mL of CH₂Cl₂ and allowed to dry overnight.

### EXAMPLE 49

Photomask polymerization of [G2]-PGLSA-MA—Gels were prepared by dissolving [G2]-PGLSA-MA, DMPA, and VP (1,000:10:1 respectively) in CH₂Cl₂ and the solution was concentrated. Next, a small amount of the polymer (with initiator and accelerator) was dissolved in a minimal amount of CH₂Cl₂ to allow spin coating of a glass cover slip. A photo mask was placed on top of this cover slip and exposed to UV light from a UVP BLAK-RAY long wave ultraviolet lamp for 15 minutes. The surface was washed with 1.0 mL of hexane and allowed to dry overnight. Biodendritic gel lines of 100 microns were formed and observed by SEM. Atomic force microscopy (AFM) shows the film to be smooth and uniform with no appreciable defects at 50 nm resolution. The RMS average of height deviation is approximately 1.5 nm

### EXAMPLE 50

Macroporous dendritic gels. Polystyrene beads of a desired size (e.g., 1 minron) were first isolated from aqueous suspension by centrifugation in an Eppendorf microfuge tube. Next the photocrosslinkable biodendritic macromolecule G2-PGLSA-MMA and the photoinitiator (DMAP) were added (with a volume specific to the desired concentration) to the Eppendorf, and mixed with the beads on a vortex spinner. The sample was then photocrosslinked with an UV lamp and removed from the eppendorf tube. The crosslinked polymer containing the polystyrene beads was then submerged in toluene for approximately 72 hours to dissolve the beads. The macroporous biomaterials were then rinsed with copious amounts of ethanol and water, and stored until further use. Scanning electron micrographs of the macroporous biomaterials show a honey-comb structures produced from a cubic closed packed arrangement of the polystyrene beads in the biopolymer prior to photocrosslinking and bead dissolution.

### EXAMPLE 51

Multiphoton fabrication of gels. In two-photon polymerization, laser excitation of a photoinitiator proceeds through at least one virtual or non-stationary state. The photo-initiator will absorb two near-IR photons, driving it into the S₂ state, followed by decay to the S₁ and intersystem crossing to the long-lived triplet state. When the spatial density of the incident photons is high, the initiator molecule (in the triplet state) will abstract an electron from TEA thus start the photocrosslinking reaction of the polymer to create the scaffold. Importantly, complex and detailed structures may be fabricated with high precision since 2-photon absorption is extremely localized under narrow focusing conditions. Controlled microfabrication via 2-photon-induced polymerization (TPIP) was used to synthesize biomedically useful structures from a solution of biopolymers. TPIP was performed using a femtosecond near-IR titanium sapphire laser (Coherent 900-F) coupled to a laser scanning confocal microscope. The average power and wavelength used for TPIP were 50 mW and 780 nm, respectively. The microscope was equipped with scanning mirrors for point and raster scans. Approximately 20 μL of solution (biopolymer, eosin y (EY), and triethanolamine (TEA), 10000:1000:1) was used as a co-initiator were dropped onto a glass microscope slide before loading onto the microscope stage for laser irradiation. A simple cross-pattern was constructed.

### EXAMPLE 52

Biodenderitic fibers. Biodendritic fibers were prepared by photo-polymerizing a solution of the crosslinkable biodendrimers while pulling the polymer from bulk solution. Scanning electron micrographs of the show well-defined fibers of micron width. By changing the concentation, photopolymerization, and extrusion rates, different fibers can be formed.

### EXAMPLE 53

Cell seeding on biodendritic gels. Photocrosslinked gels from a G2-PGLSA-MMA were from in the bottom of a 96 well plate by adding approximately 20 ul of polymer and photocrosslinking for 10 minutes with a UV-lamp as described previously. Stem cells in the appropriate media were then added to the 96 well plate. The stem cells were monitored by light microscopy at specific time intervals for 48 hours. The stem cells were alive and attached to the crosslinked biodendritic gel.

### EXAMPLE 54

Sealing a corneal transplant with a photocrosslinkable dendritic polymer. A 5.5 mm central corneal trephination will be performed in an enucleated donor human eye. A bed of viscoelastic Healon will then be introduced into the anterior chamber to help stabilize the autograft. The sterile photocrosslinkable biodendritic pol;ymer is applied to the graft-host junction with a 27 gauge cannula (N=5). The solution will then be polymerized using a continuous wave Argon laser operating at a wavelength of 514 nm and at 51 W/cm². Bursting pressures for all eyes were determined with water-column manometry employing a 23 gauge intraocular cannula connected to a reservoir of balanced salt solution at a known height above the limbus of the grafted eyes. As a reference, 10 corneal buttons will be sutured into its original position using 16 conventional interrupted 10-0 nylon sutures, without any photocrosslinkable polymer used. The bursting pressure was higher for the corneal transplant sealed with the photocrosslinkable biodendritic polymer compared to the conventional nylon suture.

### EXAMPLE 55

Syntheseis of BGL-GA-PHE-OH—Phenylalanine ethyl ester HCl (1.2 eq), BGL-GA (1 eq), and HOBt (1.2 eq) were dissolved in dry CH₂Cl₂. TEA (1.2 eq) and DCC (1.2 eq) were added and the reaction was stirred at ambient temperature overnight. DCU was removed via filtration and diluted with CH₂Cl₂ (100 mL). The product was then washed with 3.5% HCl (130 mL), water (2×130 mL), dried, and the solvent was removed. Phenylalanine ethyl ester HCl was stirred along with 0.2 M LiOH (aq) at 45° C. for two hours. The aqueous layer was acidified to pH 4, extracted with CH₂Cl₂, dried, and the solvent was removed to yield a fluffy white product. 66% overall yield. ¹H NMR and IR obtained.

### EXAMPLE 56

Synthesis of G0-PGLGAPHE-Bzld—BGL (1 eq), BGLGAPHE-OH (1.1 eq), and DPTS (0.5 eq) were dissolved in methylene chloride and the DCC (1.1 eq) was added. The reaction was stirred at ambient temperature overnight. DCU was removed via filtration and solvent removed. DPTS precipitated in EtOAc and removed via filtration. Purified with via column chromatography with 1:5 EtOH/CH₂Cl₂. Precipitated in EtOH to removed acid. 80% yield. ¹H NMR and IR obtained SEC Mw 508 PDI 1.01

### Example 57

Synthesis of G0-PGLGAPHE-OH—G0-Bzld was dissolved in THF, Pd(OH)₂ added, and was placed on hydrogenator at 80 psi for one hour. Carbon removed by filtration through a bed of celite and solvent was removed. 96% yield. ¹H NMR and IR obtained. SECMw 416 PDI 1.01

### EXAMPLE 58

Synthesis of G1-PGLGAPHE-Bzld—G0-OH (1 eq) was dissolved in DMF. Acid (5 eq) and DPTS (2.5 eq) were added, followed by DCC (5 eq). The reaction was then stirred at ambient temperature overnight. DCU was removed via filtration, and the solvent was removed on high vac. The product was then washed with ether, dissolved in EtOAc, the DPTS was removed via filtration. The product was then dissolved in minimal EtOH, and precipitated overnight in the freezer. Finally, product was purified via column chromatography with 5:1 CH₂Cl₂/EtOH. 71% yield. ¹H NMR and IR obtained SEC Mw 1704 PDI 1.01

### EXAMPLE 59

Synthesis of G1-PGLGAPHE-OH—G1-Bzld was dissolved in THF, Pd(OH)₂ added, and was placed on hydrogenator at 80 psi for 1.5 hours. Carbon removed by filtration through a bed of celite and solvent was removed. 98% yield. ¹H NMR and IR obtained. SEC Mw 1671 PDI 1.01

### EXAMPLE 60

Synthesis of G2-PGLGAPHE-Bzld—G1-OH (1 eq) was dissolved in DMF. Acid (16 eq) and DPTS (16 eq) were added, followed by DCC (16 eq). The reaction was then stirred at ambient temperature for 48 hours. DCU was removed via filtration, and the solvent was removed on high vac. DPTS was precipitated in EtOAc and removed via filtration. Purified via column chromatography with 15% EtOH in methylene chloride. Product washed with EtOH. Yield above 25% ¹H NMR and IR obtained. SEC 3681 PDI 1.01

### EXAMPLE 61

Synthesis of G2-PGLGAPHE-OH—G2-Bzld was dissolved in THF/MeOH, Pd(OH)₂ added, and was placed on hydrogenator at 80 psi for 12 hours. Carbon removed by filtration through a bed of celite and solvent was removed. 95% yield. ¹H NMR and IR obtained.

### EXAMPLE 62

### Synthesis of 2(cis-1,3-O-Benzylidene glycerol)succinic Acid Monoester (2)

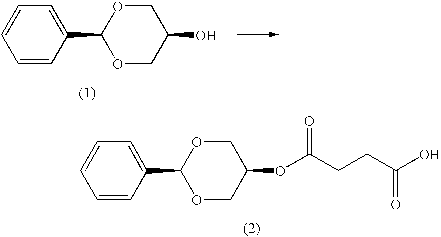

17.00 g (0.09434 mol) of cis-1,3-O-benzylidene glycerol (1) and 14.42 g (0.1441 mol) of succinic anhydride were stirred in pyridine at RT for 18 h. The pyridine was removed and the white powder was dissolved in dH₂O. The pH of the water was adjusted to 7.0 with 1 N NaOH. The water layer was washed with CH₂Cl₂ to remove impurities. The water layer was then adjusted to pH 4.0 with 1 N HCl. The product was extracted with CH₂Cl₂, dried over Na₂SO₄, filtered, and dried to yield 25.023 g of pure product as a white powder (94.6% yield). ¹H ¹H NMR and IR obtained GC-MS: 281 m/z (MH⁺) (theory: 280 m/z (M⁺)). Elemental analysis: C, 60.07%; H, 5.80% (theory: C, 59.99%; H, 5.75%).

### EXAMPLE 63

### Synthesis of cis-1,3-O-benzylidene-2-O-(succinate methylphthalimide) Glycerol (bzld-G1-PGLSA-phth Dendron) (3)

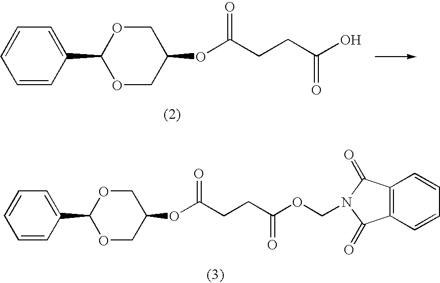

4.004 g (0.01429 mol; 1 equiv) of cis-1,3-O-benzylidene-2-O-(succinic acid) glycerol (2) and 3.803 g (0.01584 mol; 1.1 equiv) of N-bromomethylphthalimide and 2.002 g (0.03446 mol; 2.4 equiv) of potassium fluoride stirred in DMF at 85° C. for two hours. The DMF was then removed under vacuum. The solid product was dissolved in CH₂Cl₂, washed with water, sat. NaHCO₃, dried over Na₂SO₄, rotovapped and precipitated in ether. The final product was recrystallized in MeOH for 4.169 g of a white powder in 66.5% yield. ¹H NMR and IR obtained GC-MS: 440.1 m/z (MH⁺) (theory: 439.4 m/z (M⁺)).

### EXAMPLE 64

### Benzylidene Deprotection of cis-1,3-O-benzylidene-2-O-(succinate methylphthalimide) glycerol

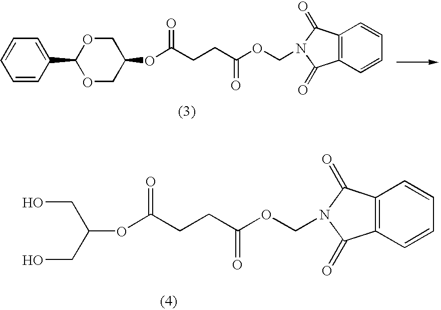

The benzylidene protecting group of cis-1,3-O-benzylidene-2-O-(succinate methylphthalimide) glycerol was removed by catalytic hydrogenolysis. 2.00 g of cis-1,3-O-benzylidene-2-O-(succinate methylphthalimide) glycerol was dissolved in EtOAc/MeOH (9:1) and 10% w/w 10% Pd/C was added. The solution was then placed in a Parr tube on a hydrogentator and shaken under 50 atm H₂ for 1 h. The solution was then filtered over wet celite. The product was purified by column chromatography (CH₂Cl₂:MeOH 95:5) for 1.5 g of clear oil (94% yield). ¹H NMR and IR obtained.

### EXAMPLE 65

### Synthesis of DPTS

DPTS was synthesized according to the procedure of Moore and Stubb [Moore, 1990 #197] Para-toluene sulfonic acid (PTSA) was dissolved in toluene and dried on a vacuum line. It was dissolved in dry toluene at 40° C. An equimolar amount of DMAP (4-dimethyl amino pyridine; 122.17 g/mol) was dissolved in warm toluene and added to the solution. The solution was stirred overnight and a white solid precipitated. The solution was filtered. The precipitate was dried on the vacuum line and used without further purification. This is a 1:1 salt complex of para-toluene sulfonic acid and 4-dimethylaminopyridine with a melting point of 165° C.

### EXAMPLE 66

### Synthesis of Benzylidene-G-2 PGLSA-Methylphthalimide Dendron

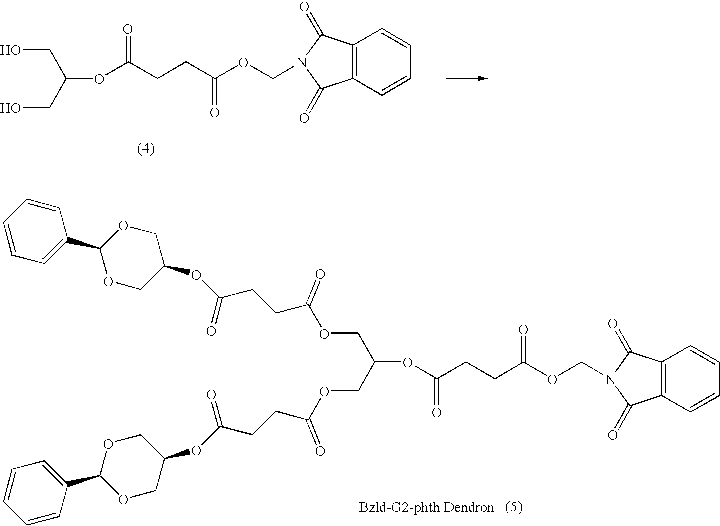

1.50 g (4.27 mmol) of the deprotected product was stirred in dry CH₂Cl₂ with 2.63 g (9.38 mmol, 2.2 equiv) of cis-1,3-O-benzylidene-2-O-(succinic acid) glycerol, 1.26 g (4.28 mmol, 1 equiv) DPTS, and 2.64 g (12.8 mmol, 3 equivalents) of DCC at RT overnight. The solution was filtered, rotovapped and placed in cold THF, filtered again, rotovapped, recrystallized in ether, filtered, and purified by column chromatography (CH₂Cl₂ to CH₂Cl₂:MeOH 95:5) to produce 3.23 g (3.69 mmol) of white powder (86% yield). ¹H NMR and IR obtained GC-MS: 876.3 m/z (MH⁺), (theory: 875.3 m/z (M⁺)). HR-FAB: 874.2537 m/z (M−H⁺) (theory: 875.2637 m/z (M⁺)).

### EXAMPLE 67

### Benzylidene Deprotection of Bzld-G2-PGLSA-phth Dendron

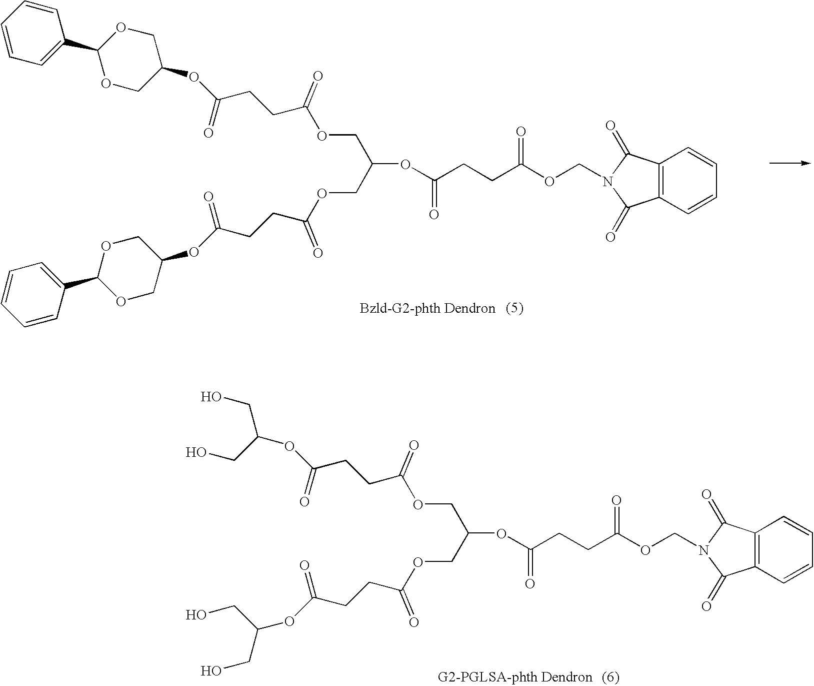

0.746 g of Bzld-G2-phth dendron (5) was dissolved in THF. 10% w/w of 10% Pd/C was added to the solution which was subsequently placed on the hydrogenator under 40 atm H_{2(g)} for 1 h. The solution was filtered over celite and dried resulting in 0.52 g (0.743 mmol) of oily product (6) in a 95% yield. ¹H NMR and IR obtained

### EXAMPLE 68

### Synthesis of Bzld-G3-PGLSA-phth Dendron (7)

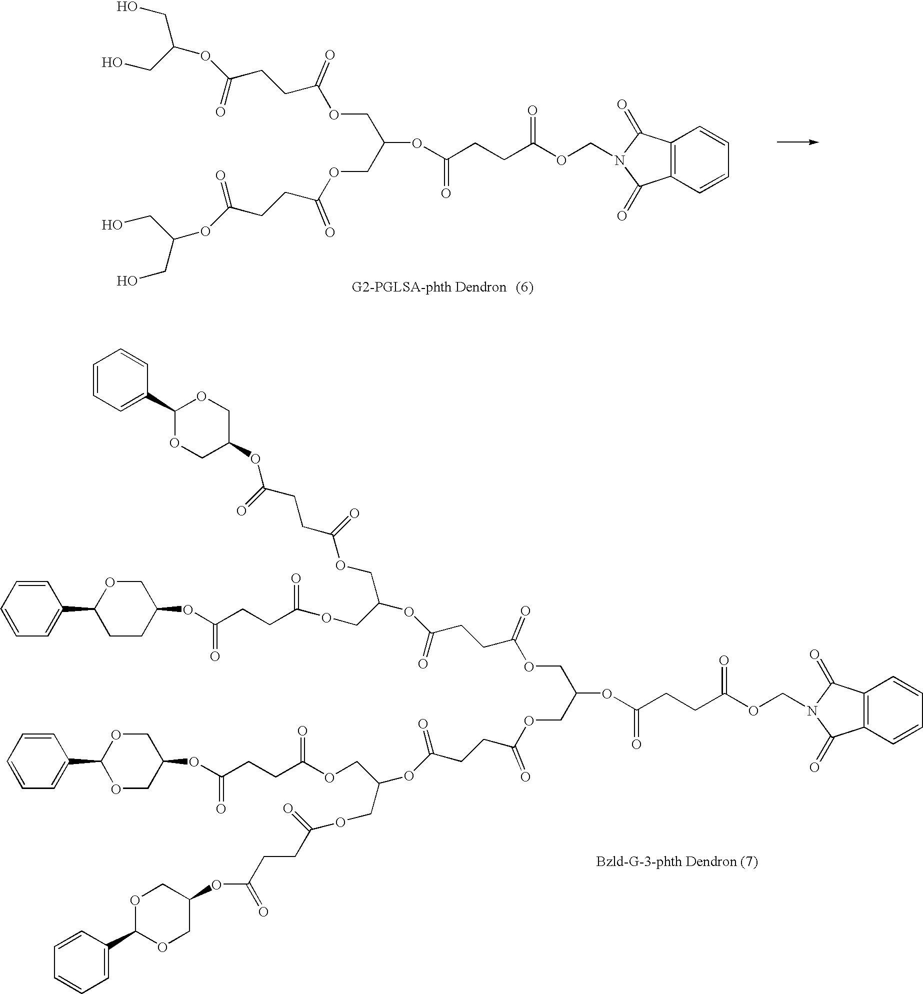

0.52 g of benzylidene deprotected G2-PGLSA-phth dendron (6) (0.743 mmol) was dissolved in dry CH₂Cl₂. 0.916 g of cis-1,3-O-benzylidene-2-O-(succinic acid) glycerol (2) (3.27 mmol; 4.4 equiv), 0.44 g (1.44 mmol) DPTS, and 0.674 g (3.27 mmol) DCC were added. The reaction was stirred overnight at RT. It was filtered to remove the DCU that was produced, purified in cold THF to further remove DCU and recrystallized in cold ether. The product was purified by column chromatography (95:5 CH₂Cl₂:MeOH; R_{f}=0.82) resulting in a solid white powder (7) in a 84% yield. ¹H NMR and IR obtained GC-MS: 1749.5 m/z (MH⁺) (theory: 1748.7 m/z (M⁺)). Elemental analysis: C, 59.17%; H, 5.56% (theory: C, 59.07%; H, 5.36%). SEC: M_{w}=1880, Mₙ=1850, PDI=1.01.

### EXAMPLE 69

### Synthesis of cis-1,3-O-benzylidene-2-O-(succinate(t-butyl-diphenyl silyl))glycerol(bzld-G1-GLSA-Si Dendron) (9)

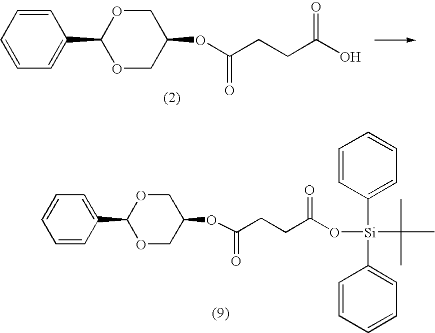

4.002 g (0.0143 mol) of cis-1,3-O-benzylidene-2-O-(succinic acid) glycerol (2) and 3.24 g (3.3 equiv of imidazole) were stirred in a small amount of DMF. 6.4 mL (1.7 equiv) of diphenyl-t-butyl silyl chloride were added and the reaction was stirred at 25° C. for 48 h. CH₂Cl₂ was added and washed with sat. NaHCO₃ and water, dried over Na₂SO₄, filtered, rotovapped, and dried on vacuum line. The product was purified by column chromatography (4:1 hexanes:EtOAc) resulting in 6.38 g (0.123 mol) of product as a thick opaque oil (9) (86.1% yield). R_{f}=0.130 in 4:1 hexanes:EtOAc. ¹H NMR and IR obtained. GC-MS: 519.2 m/z (MH⁺) (theory: 518.7 m/z (M⁺)). HR-FAB: 517.2028 m/z (M−H⁺) (theory: 518.2125 m/z (M⁺)).

### EXAMPLE 70

### Benzylidene Removal of bzld-G1-GLSA-Si Dendron (10),

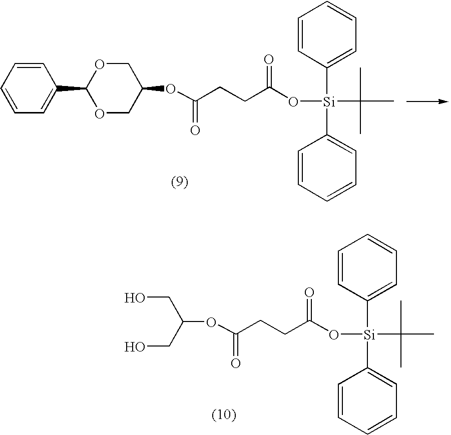

1 equivalent of cis-1,3-O-benzylidene-2-O-(succinate (diphenyl-t-butyl silyl)) glycerol was dissolved in THF, 10% w/w 10% Pd/C was added. The solution was then placed in a Parr tube on a hydrogentator, evacuated, flushed with hydrogen, and shaken under 40 atm H₂ for 3 hours. The solution was then filtered over wet celite. Rotovapped and purified by column chromatography (1:1 Hex:EtOActo 1:4 Hex:EtOAc). ¹H NMR and IR obtained.

### EXAMPLE 71

### Synthesis of bzld-G2-PGLSA-Si Dendron (11)

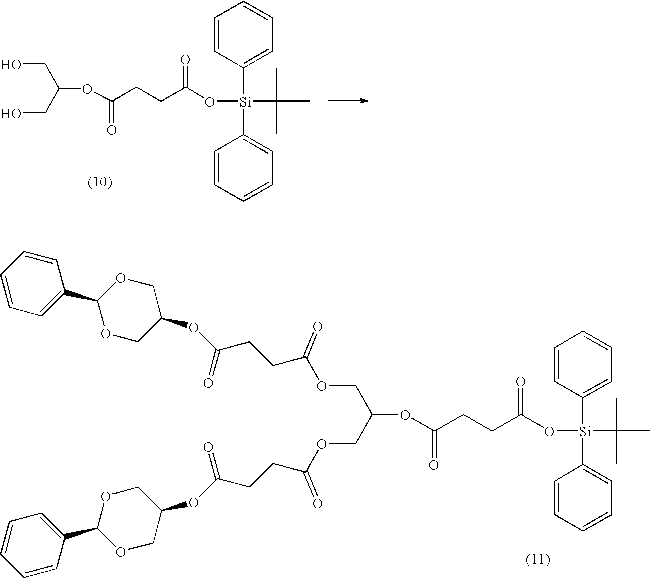

1.90 g (4.41 mmol) of 2-O-(succinate (diphenyl-t-butyl silyl)) glycerol was stirred in dry CH₂Cl₂, 1.30 g (1 equiv; 4.41 mmol) DPTS, 2.72 g (9.70 mmol; 2.2 equiv) of cis-1,3-O-benzylidene-2-(succinic acid) glycerol, and 2.00 g (9.70 mmol; 2.2 equiv) of DCC were added. The solution was stirred at RT overnight (within 15 minutes DCU begins to precipitate out). The DCU precipitate was filtered off and the solution was evaporated. A solution of 1:1 ethyl acetate:hexanes was added and the impurities crash out, while the product (G-2 Dendron) remains in solution. The solution was filtered rotovapped and placed on the vacuum line and purified by column chromatography (1:1 hexanes:EtOAc), for 3.70 g (3.87 mmol) of product (88% yield). R_{f}=0.2155 (1:1 hexanes:EtOAc); R_{f}=0.5091 (3:7 hexanes:EtOAc). ¹H NMR and IR obtained. GC-MS: 955.3 m/z (MH⁺) (theory: 955.1 m/z (M⁺)). SEC: M_{w}=940, Mₙ=930, PDI=1.01.

### EXAMPLE 72

### Benzylidene removal of bzld-G2-PGLSA-Si Dendron (11) to Yield G2-PGLSA-Si Dendron (12).

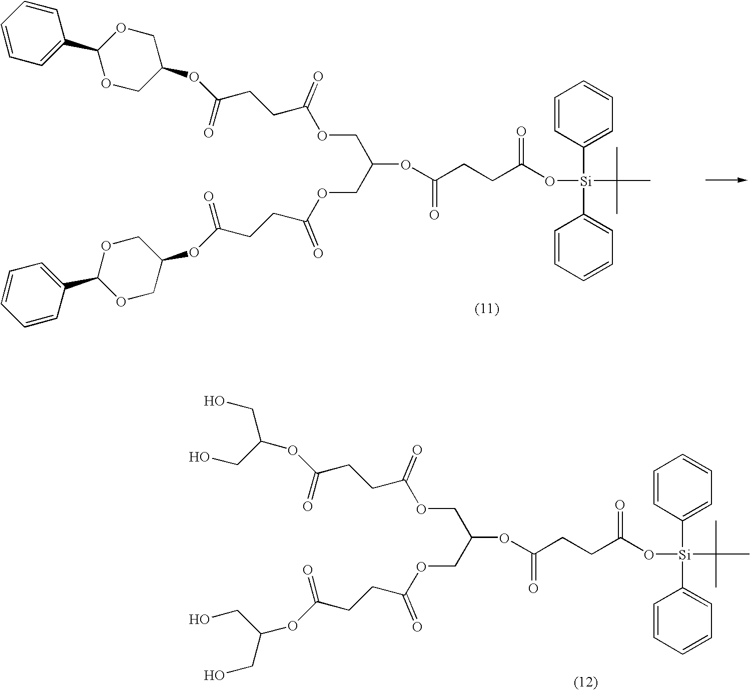

1.55 g (1.62 mmol) of bzld-G2-PGLSA-Si dendron (11) was dissolved in THF, excess 20% Pd(OH)₂/C was added. The solution was then placed in a Parr tube on a hydrogentator and shaken under 50 atm H₂ for 4 hours. The solution was then filtered over wet celite, rotoevaporated, and purified by column chromatography (1:1 Hex:EtOAc to 1:4 Hex:EtOActo yield 1.12 g (1.54 mmol) of benzylidene deprotected G2-PGLSA-Si dendron (12) (95% yield). ¹H NMR and IR obtained.

### EXAMPLE 73

### Silyl Removal from bzld-G2-PGLSA-Si Dendron (11) to yield bzld-G2-PGLSA Dendron (14)

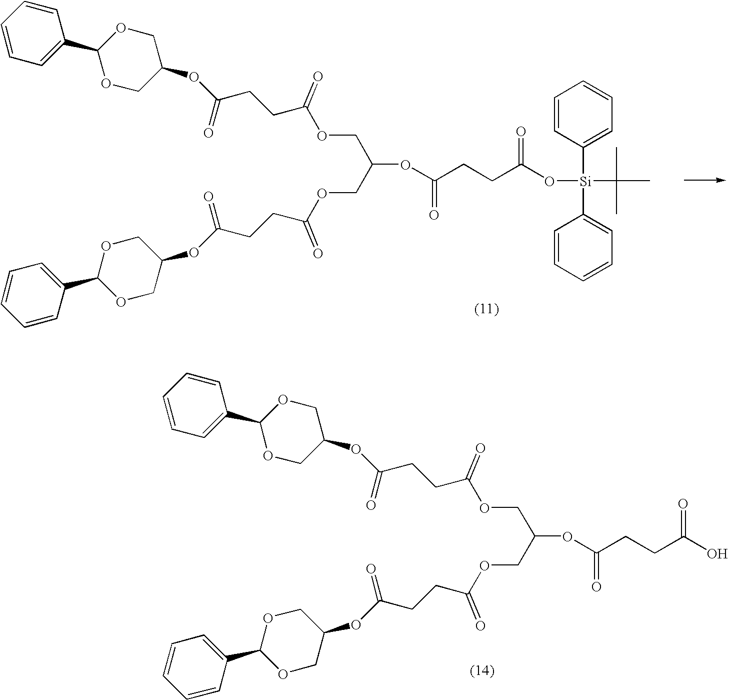

1.00 g (1.04 mmol) of bzld-G2-PGLSA-Si dendron (11) was dissolved in THF. 1.25 g (3.96 mmol; 3.8 equiv) of tetrabutylammonium fluoride hydrate, (TBAF 3H₂O; 315.51 g/mol) was added to the solution and it was stirred at RT for 1 hour. After one hour the reaction was complete, as evidenced by TLC. The solution was washed 2× with H₂O, dried over Na₂SO₄, rotoevaporated and dried on the vacuum line. The product was purified by column chromatography (100% CH₂Cl₂ to 2% MeOH in CH₂Cl₂) for 0.65 g (0.907 mmol; 87% yield) of product (14). ¹H NMR and IR obtained. GC—SEC: M_{w}=810, Mₙ=800, PDI=1.01.

### EXAMPLE 74

### Synthesis of bzld-G3-PGLSA-Si Dendron (13)

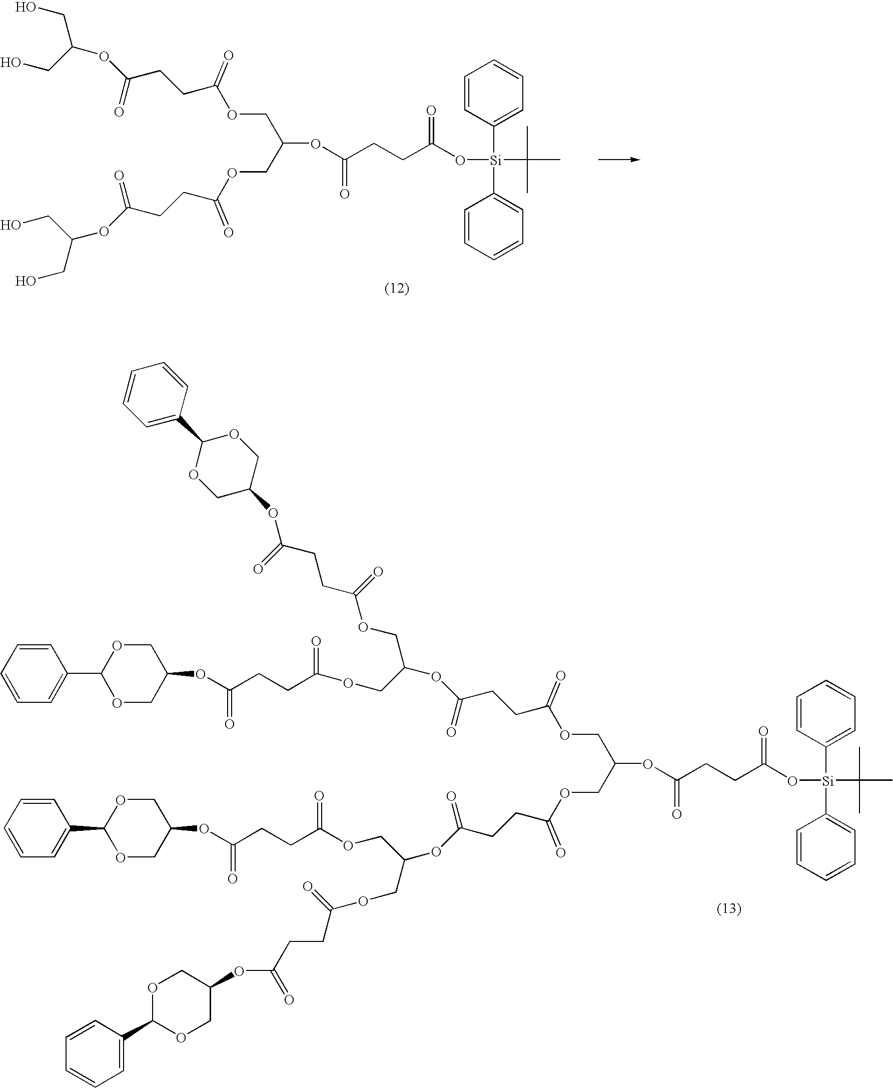

0.55 g (0.71 mmol) of benzylidene deprotected G2 dendron (12) was stirred in dry CH₂C[2, 0.415 g (1.41 mmol; 2 equiv.) DPTS, 0.871 g (3.11 mmol; 4.4 equiv) of cis-1,3-O-benzylidene-2-(succinic acid) glycerol monoester (2), and 4.4 equivalents DCC were added. The solution was stirred under nitrogen at RT overnight (within 15 minutes DCU begins to precipitate out). The DCU precipitate was filtered off and the solution was evaporated. The product was purified by column chromatography (3:7 hexanes:EtOAc) with a yield of 0.71 g of (13) (54% yield). ¹H NMR and IR obtained. GC-MS: 1825.6 m/z (M−H⁺) (theory: 1827.9 m/z (M⁺)). HR-FAB: 1825.6124 m/z (M−H⁺) (theory: 1826.6233 m/z (M⁺)). SEC: M_{w}=1830, Mₙ=1810, PDI=1.01.

### EXAMPLE 75

### Silyl Removal from bzld-G3-PGLSA-Si Dendron (13) to Yield bzld-G3-PGLSA (8)

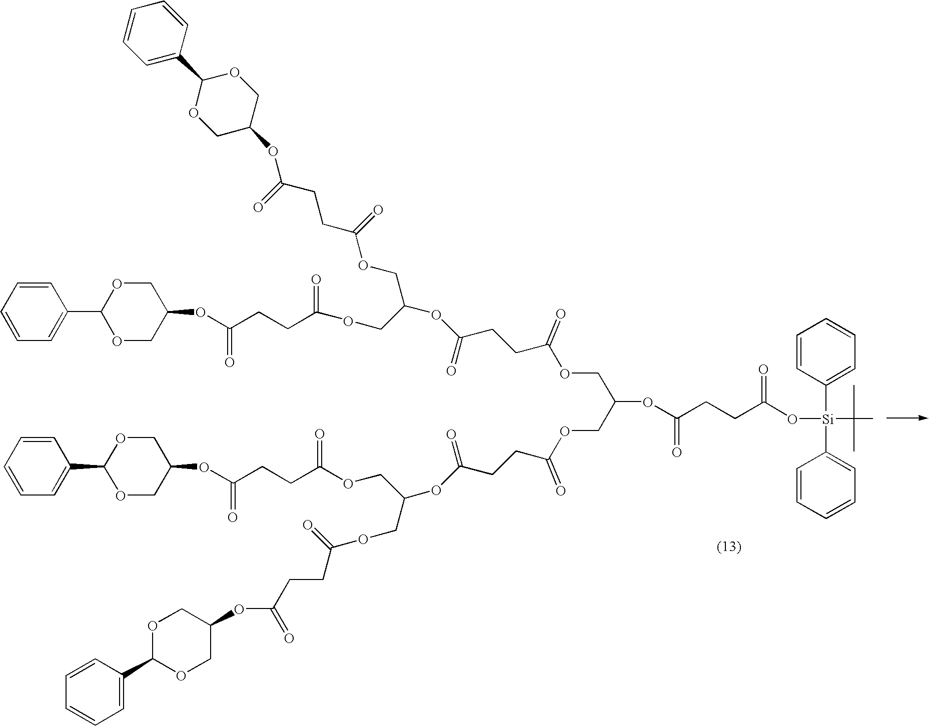

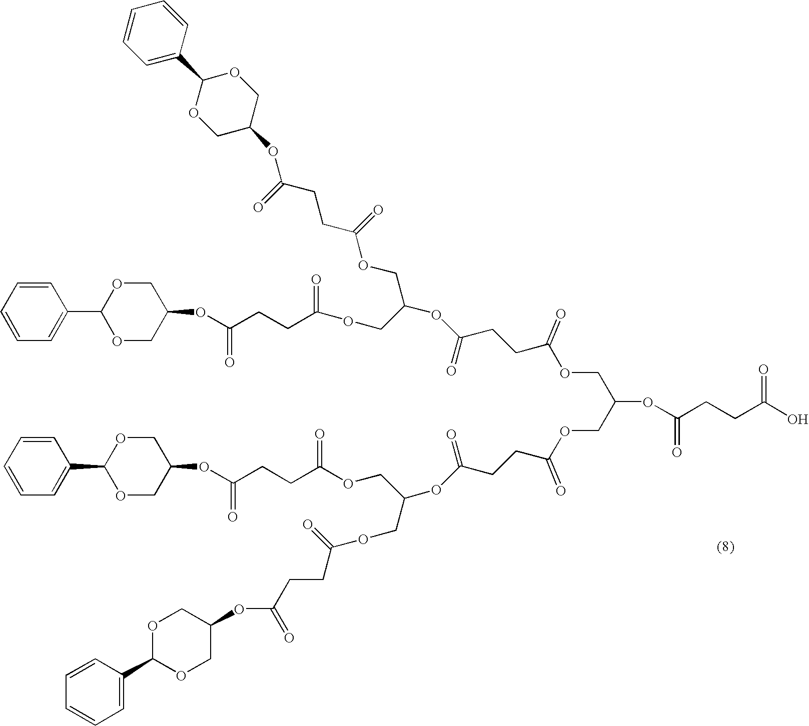

The t-butyl-diphenyl silyl group was removed from the G3 dendron and the product was purified in an analogous manner as the G2 dendron. 2.00 g (1.09 mmol) of bzld-G3-PGLSA-Si dendron (13) was dissolved in THF. 1.3 g (4.1 mmol; 3.8 equiv) of tetrabutylammonium fluoride hydrate, (TBAF 3H₂O; 315.51 g/mol) was added to the solution and it was stirred at RT for 1 hour. After one hour the reaction was complete, as evidenced by TLC. The solution was washed 2× with H₂O, dried over Na₂SO₄, rotoevaporated and dried on the vacuum line. The product was purified by column chromatography (100% CH₂Cl₂ increasing to 2% MeOH in CH₂Cl₂) for 1.44 g (0.906 mmol; 83% yield) of product (17). ¹H NMR and IR obtained. SEC: M_{w}=1650, Mₙ=1620, PDI=1.02, M_{actual}=1589.50.

### EXAMPLE 76

### Benzylidene Removal from bzld-G3-PGLSA-Si Dendron

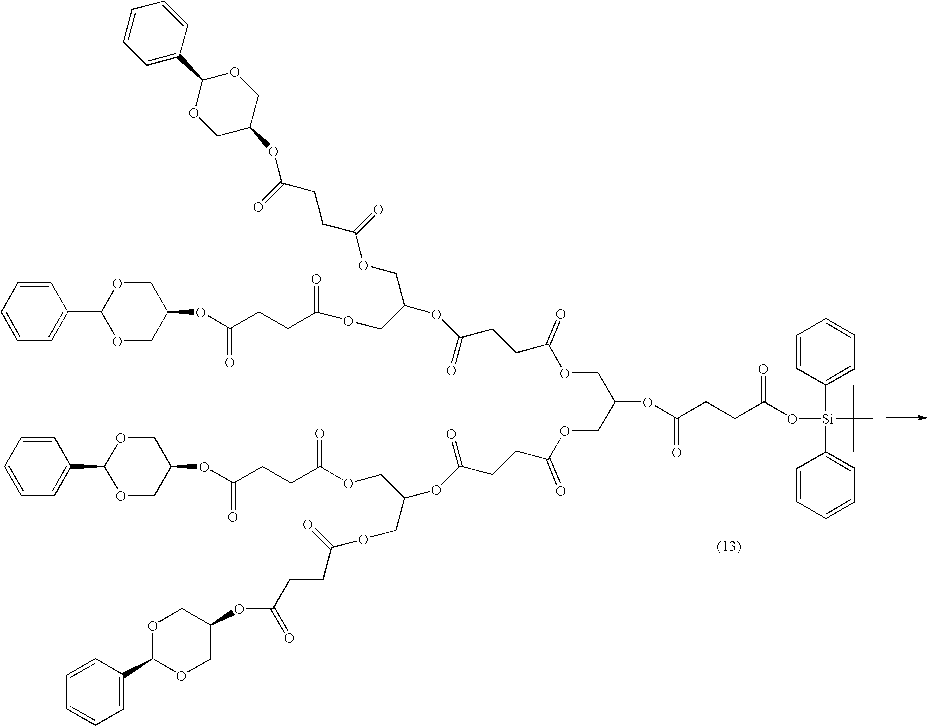

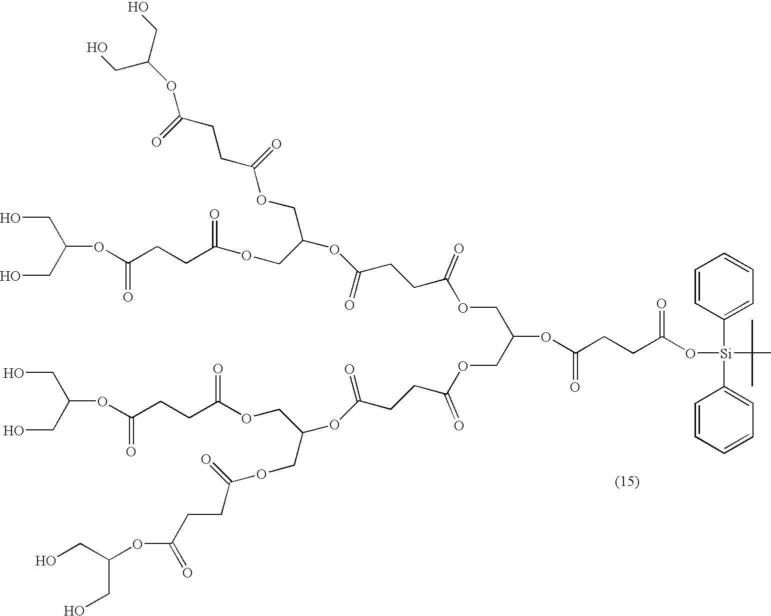

0.484 g bzld-G3-PGLSA-Si Dendron (13) dissolved in THF. 20% Pd(OH)₂ was added and the flask was evacuated and filled with 50 psi H₂. The mixture was shaken for 1 hour, then filtered over celite. The filtrate was dried to produce an oil in 0.38 g or 97% yield. ¹H NMR and IR obtained.

### EXAMPLE 77

### Synthesis of bzld-G4-PGLSA-Si Dendron (16)

The bzld-G4-PGLSA-Si dendron was synthesized by two methods, by the addition of monoester (2) to G3-PGLSA-Si dendron (without bzld) (15) by DCC coupling (G3+G1 method) or by the addition of bzld-G2-PGLSA (without Si) (14) to G2-PGLSA-Si (without bzld) (12) also by DCC coupling for a G2+G2 method. See Scheme 4.4 for a depiction of both methods.

G3+G1: 0.38 g of G3-PGLSA-Si (0.26 mmol) was dissolved in dry DCM. 1.00 g (3.57 mmol) of cis-1,3-O-benzylidene-2-(succinic acid) glycerol monoester (2), 0.10 g (0.34 mmol) DPTS, and 0.656 g (3.57 mmol) g DCC were added to the mixture. The solution was stirred 48 h under nitrogen at RT. The DCU precipitate was filtered off and the filtrate was dried and purified by column chromatography (1:1 hexanes:EtOAc to 1:4 hexanes:EtOAc). 0.572 g (0.16 mmol) of a white hydroscopic powder (16) was isolated in 60% yield. ¹H NMR and IR obtained. MALDI-MS: 3574.54 m/z (MH⁺) (theory: 3573.54 m/z (M⁺)). SEC: M_{w}=3420, Mₙ=3350, PDI=1.02.

### EXAMPLE 78

### Synthesis of PGLSA Dendrimer Tetrafunctional Core (bzld-G0-PGLSA) (17)

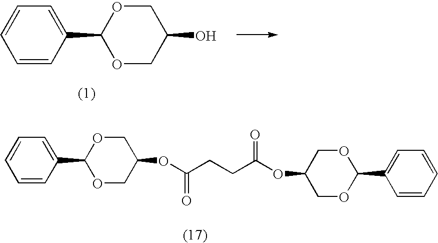

Succinic acid, cis-1,3-O-benzylidene glycerol and DPTS were dissolved in dry CH₂Cl₂. DCC was added and the reaction was stirred under nitrogen at RT overnight. The DCU was filtered off, and the filtrate was concentrated and purified by column chromatography (97:3 CH₂Cl₂:MeOH). 90% yield. ¹H NMR and IR obtained. GC-MS: 443 m/z (MH⁺) (theory: 442 m/z (M⁺)). HR-FAB: 442.1635 m/z (M⁺) (theory: 442.1628 m/z (M⁺)). Elemental analysis: C, 65.25%; H, 5.85% (theory: C, 65.15%; H, 5.92%).

### EXAMPLE 79

### Benzylidene Deprotection of Tetrafunctional Core

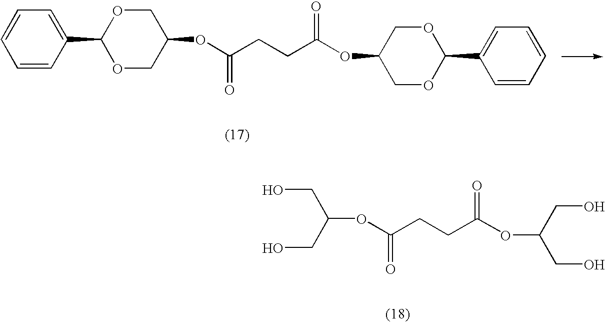

1.00 g (0.0023 mol) of bzld-G0-PGLSA (17) was dissolved in THF in a Parr tube. 10% w/w Pd(OH)₂/C was added. The Parr tube was evacuated, flushed with H_{2(g)}, and filled with 50 psi of H₂. The solution was shaken for 3 hours. The catalyst was filtered ans washed with THF. The filtrate was evaporated to give 0.57 g (0.0022 mol) of a clear oily product (95% yield). ¹H NMR and IR obtained. Elemental analysis: C, 44.94%; H, 6.87% (theory: C, 45.11%; H, 6.81%).

### EXAMPLE 80

### Synthesis of bzld-G3-PGLSA Dendrimer (19)

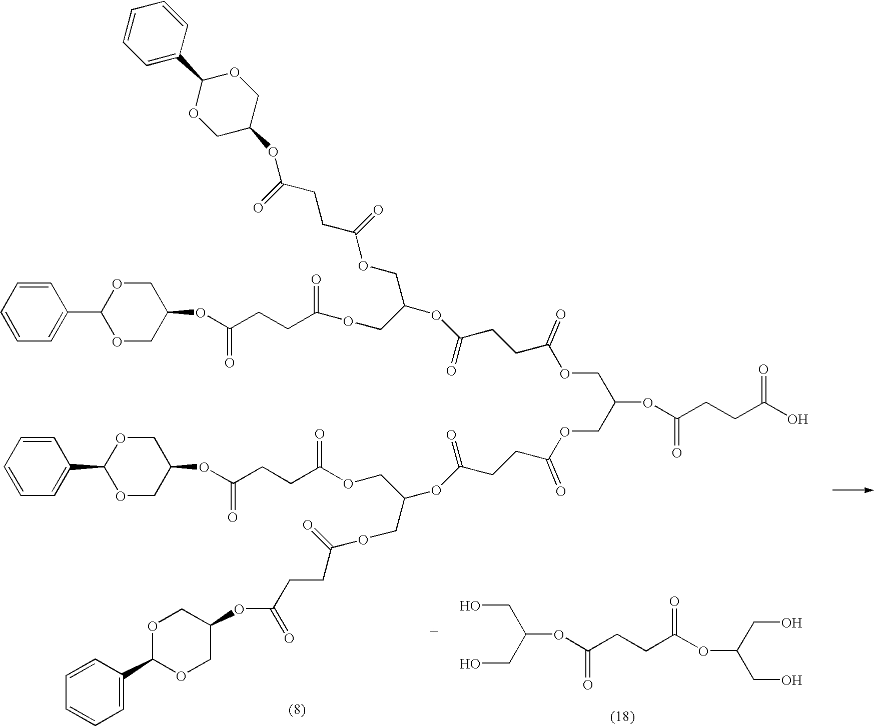

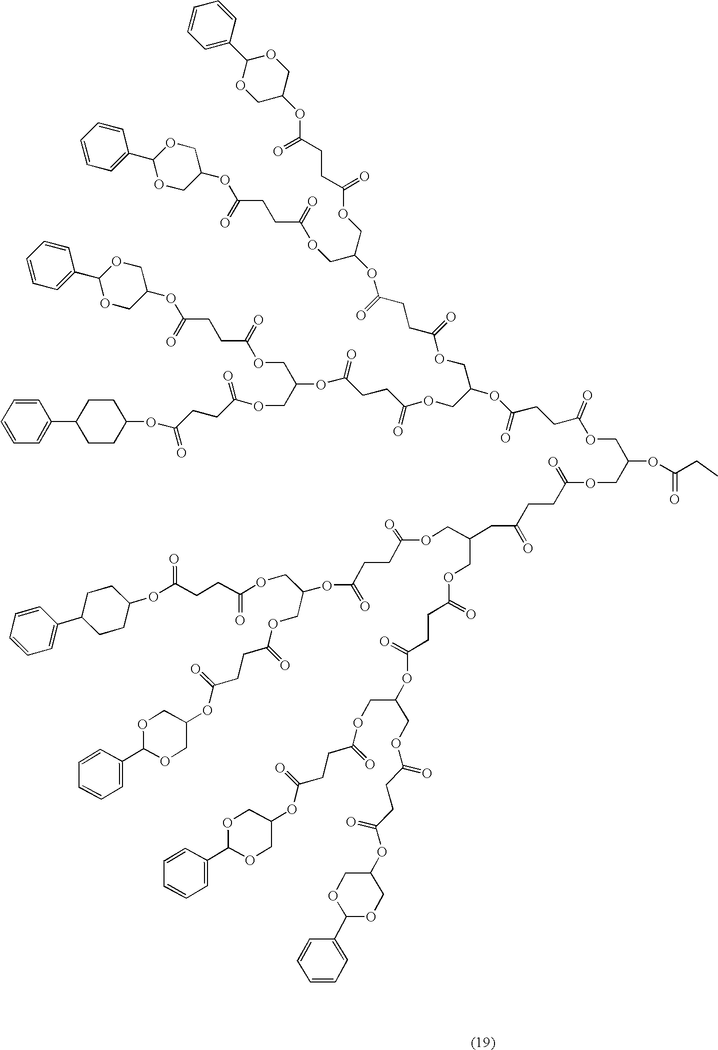

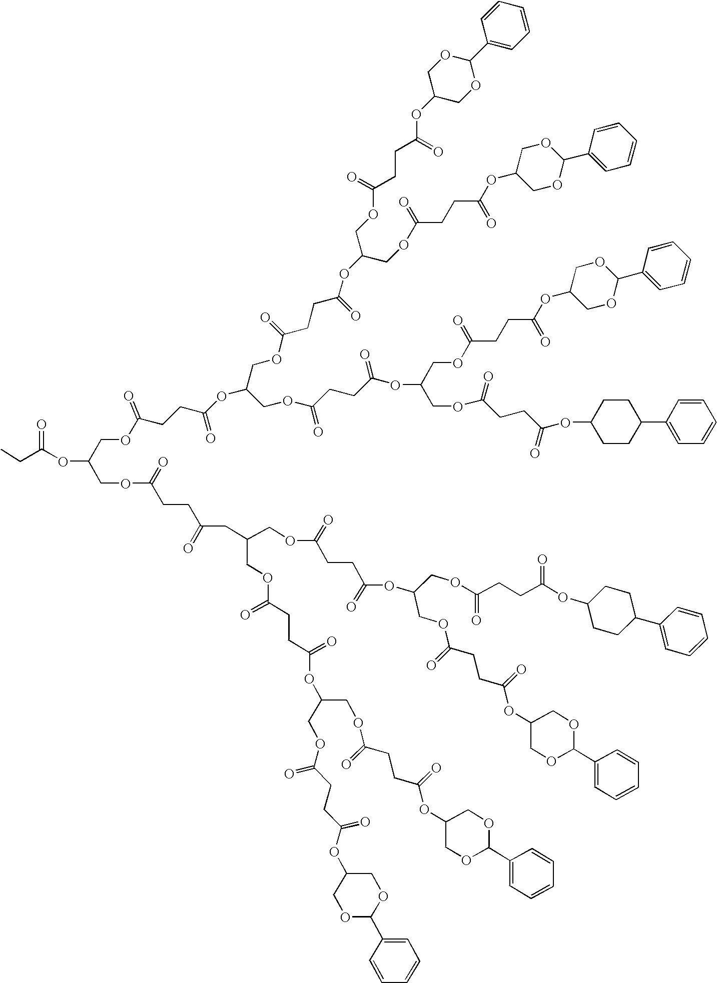

0.029 g (0.11 mmol) of tetrafunctional core (18) dissolved in dry DCM. 0.9 g (0.57 mmol) bzld-G3-PGLSA (8), 33 mg (0.11 mmol) DPTS, and 0.12 g DCC (0.57 mmol) were added. The solutions was stirred 72 h at RT under nitrogen. SEC: M_{w}=4740, Mₙ=4590, PDI=1.01, M_{theoretical}=6552.19. ¹H NMR and IR obtained.

### EXAMPLE 81

### Synthesis of (bzld-G3-PGLSA)-PEG Linear Hybrid (20)

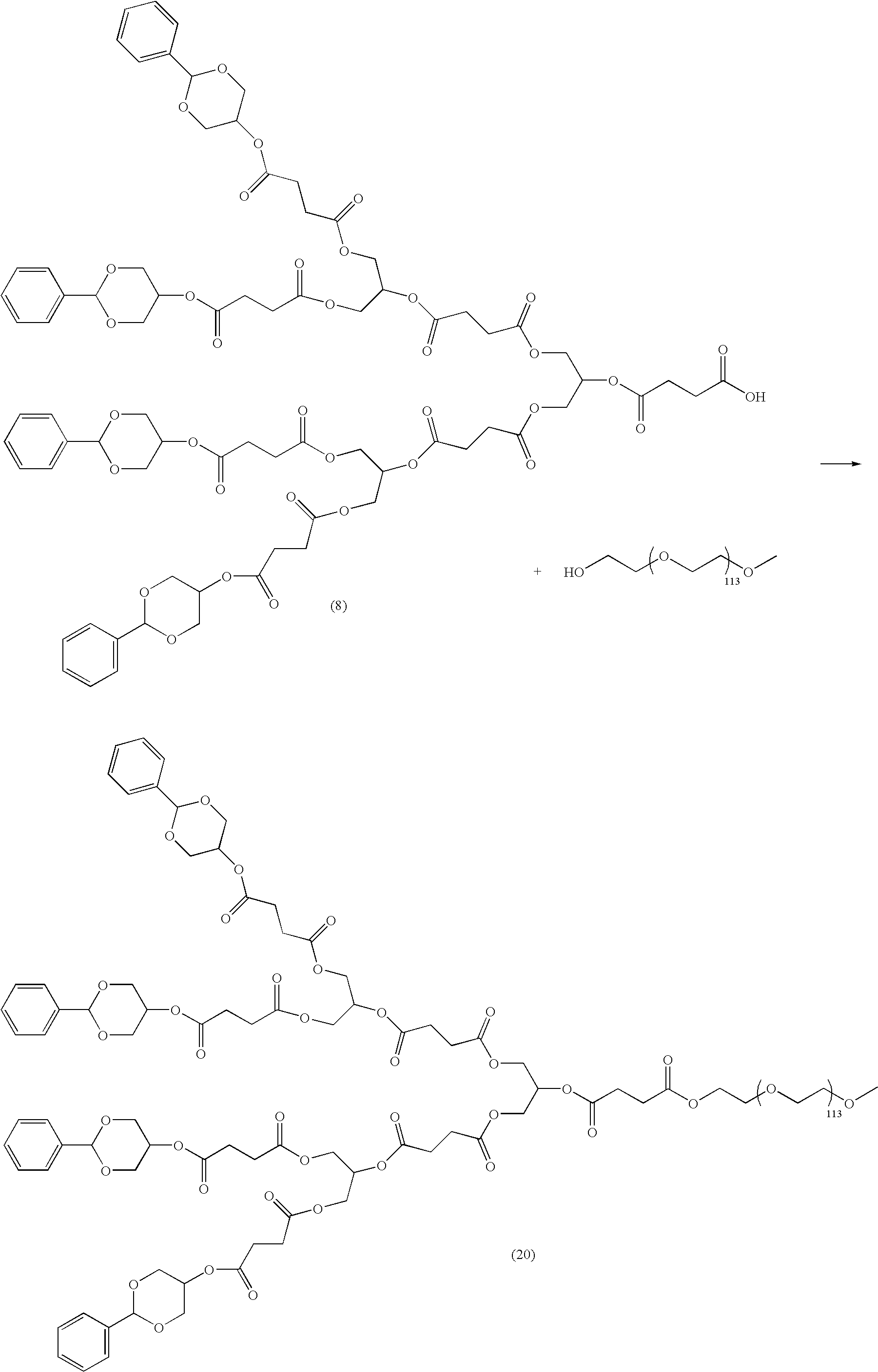

0.29 g (0.18 mmol) of bzld-G3-PGLSA dendron (8) was dissolved in dry DCM, 0.45 g (0.09 mmol) 5000 MW poly(ethylene glycol) mono-methyl ether (PEG-MME) (Polysciences, Inc., Warrington, Pa.), 0.037 g (0.18 mmol) DCC, and 0.026 g (0.09 mmol) DPTS were added to the solution. The solution was stirred under nitrogen at RT for 168 h. The DCU was filtered off. The filtrate was rotovapped and redissolved in THF, cooled, and the DCU was filtered off. The product was precipitated in ethyl ether. The solid was dissolved in THF, stirred with Amberlyst A-21 ion-exchange resin (Aldrich) (weakly basic resin) to eliminate the excess bzld-G3-PGLSA-acid (8). The solution was filtered and the filtrate was dried to yield 0.528 g of a solid white product (89% yield) (20). MALDI-MS: M_{w}=6671, Mₙ=6628 PDI=1.01 (theoretical MW=6588; PEG-MME (5000 glmol) sample: MALDI-MS M_{w}=5147, Mₙ=5074, PDI=1.01). SEC: M_{w}=6990, Mₙ=6670, PDI=1.04. ¹H NMR and IR obtained.

We claim:

1. Dendritic polymers or copolymers comprised of building blocks derived from at least one biocompatible or natural metabolite in vivo selected from the group consisting of glycerol, lactic acid, glycolic acid, glycerol, amino acids, caproic acid, ribose, glucose, succinic acid, malic acid, amino acids, peptides, synthetic peptide analogs, poly(ethylene glycol), and poly(hydroxyacids).

2. A crosslinkable/polymerizable dendritic polymer or monomer according to claim 1 for wound care or wound management.

3. A crosslinkable/polymerizable dendritic polymer or monomer according to claim 1 as a tissue sealant.

4. A crosslinkable/polymerizable dendritic polymer or monomer according to claim 1 for seeding cells in vitro for subsequent in vivo placement.

5. A crosslinkable/polymerizable dendritic polymer or monomer according to claim 1 for seeding with cells and subsequent in situ polymerization in vivo.

6. A crosslinkable/polymerizable dendritic polymer or monomer according to claim 1 for prevention of adhesion.

7. A crosslinkable/polymerizable dendritic polymer or monomer according to claim 1 for organ repair or restoration.

8. A crosslinkable dendritic polymer or monomer according to claim 1 wherein the crosslinking is of covalent, ionic, or hydrophobic nature.

9. A dendritic polymer according to claim 1 for drug delivery.

10. A dendritic polymer according to claim 1 for gene delivery.

11. A dendritic polymer according to claim 1 for medical imaging.

12. A dendritic polymer according to claim 1 for cosmetic or plastic surgery

13. A dendritic polymer according to claim 1 mixed with linear polymers for a medical or tissue engineering application.

14. A crosslinkable dendritic polymer or monomer according to claim 1 wherein the said crosslinking dendritic polymer is mixed with a one or more linear polymers.

15. A crosslinkable dendritic polymer or monomer according to claim 1 wherein the final polymeric form is a gel, film, fiber, or woven sheet.

16. A crosslinkable dendritic polymer or monomer according to claim 1 wherein the final polymeric form is produced by a single or multi-photon process.

17. A crosslinkable or noncrosslinkable polymer according to claim 1 wherein the polymer is a star biodendritic polymer or copolymer as shown in at least one of the formulas below: 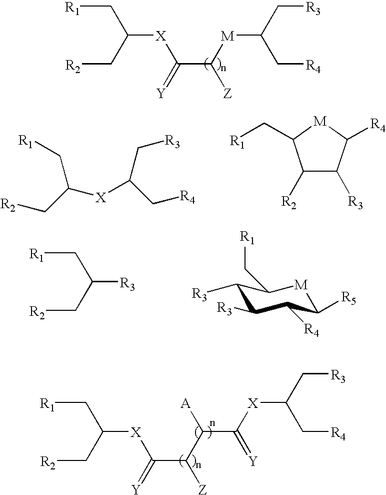
wherein R₁, R₂, R₃, R₄, R₅, A or Z, which may be the same or different, are —H, —CH₃, —OH, methoxy, carboxylic acids, sulfates, phosphates, aldehydes, amines, amides, thiols, disulfides, straight or branched chain alkanes, straight or branched chain alkenes, straight or branched chain esters, straight or branched chain ethers, straight or branched chain silanes, straight or branched chain urethanes, straight or branched chains, carbonates, straight or branched chain sulfates, straight or branched chain phosphates, straight or branched chain thiol urethanes, straight or branched chain amines, straight or branched chain thiol urea, straight or branched chain thiol ethers, straight or branched chain thiol esters, and
wherein Y, X and M, which may be the same or different, are O, S, Se, N(H) and P(H), and n is 1-50.

18. A crosslinkable or noncrosslinkable polymer according to claim 17 which is fully saturated and/or unsaturated.

19. A crosslinkable or noncrosslinkable polymer according to claim 17 wherein straight or branched chains are the same number of carbons or different, and wherein R₁, R₂, R₃, R₄, R₅, A or Z are linked by at least one linker selected from the group consisting of esters, silanes, ureas, amides, amines, urethanes, thio]-urethanes, carbonates, thio-ethers, thio-esters, sulfates, phosphates and ethers.

20. A crosslinkable or noncrosslinkable polymer according to claim 17 which includes at least one chain selected from the group consisting of hydrocarbons, flourocarbons, halocarbons, alkenes, and alkynes.

21. A crosslinkable or noncrosslinkable polymer according to claim 17 which includes at least one chain selected from the group consisting of linear and dendritic polymers.

22. A crosslinkable or noncrosslinkable polymer according to claim 21 wherein said wherein said linear and dendritic polymers include at least one selected from the group consisting of polyethers, polyesters, polyamines, polyacrylic acids, polycarbonates, polyamino acids, polynucleic acids and polysaccharides of molecular weight ranging from 200-1,000,000, and wherein said chain contains 0, 1 or more than 1 photopolymerizable group.

23. A crosslinkable or noncrosslinkable polymer according to claim 22, wherein the polyether is PEG, and wherein the polyester is PLA, PGA or PLGA.

24. A polymer of claim 22 or a linear polymer wherein the chain is a polymer or copolymer of a polyester, polyamide, polyether, or polycarbonate of: 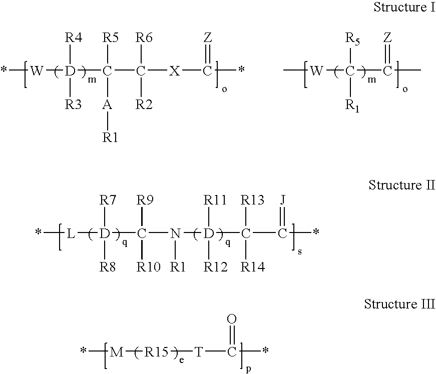
wherein R6-R15, which may be the same or different are —H, —CH₃, —OH, methoxy, carboxylic acids, sulfates, phosphates, aldehydes, amines, amides, thiols, disulfides, straight or branched chain alkanes, straight or branched chain alkenes, straight or branched chain esters, straight or branched chain ethers, straight or branched chain silanes, straight or branched chain urethanes, straight or branched chains, carbonates, straight or branched chain sulfates, straight or branched chain phosphates, straight or branched chain thiol urethanes, straight or branched chain amines, straight or branched chain thiol urea, straight or branched chain thiol ethers, straight or branched chain thiol esters, and
and wherein each of o, s and p is a number between 1 to 10000, and each of m, q, r and e is a number between 1 to 10.

25. A polymer of claim 24 comprised of repeating units of general Structure I, where A is O, S, Se, or N—R7, whrein R7 is the same as R1.

26. A polymer as in claim 24, where W, X, and Z are the same or different at each occurrence and are O, S, Se, N(H), or P(H).

27. A polymer as in claim 24, where any one of R6-R15 is hydrogen, straight or branched alkyl chains of 1-20 carbons, cycloalkyl, aryl, olefin, silyl, alkylsilyl, arylsilyl, alkylaryl, or arylalkyl groups substituted internally or terminally by one or more hydroxyl, hydroxyether, carboxyl, carboxyester, carboxyamide, amino, mono- or di-substituted amino, thiol, thioester, sulfate, phosphate, phosphonate, or halogen substituents.

28. A polymer as in claim 24, where any one of R6-R15 is a polymer selected from poly(ethylene glycols) poly(ethylene oxide), or poly(hydroxyacids, or is selected from carbohydrates, proteins, polypeptides, amino acids, nucleic acids, nucleotides, polynucleotides, DNA or RNA segments, lipids, polysaccharides, antibodies, pharmaceutical agents, or epitopes for a biological receptor.

29. A polymer as in claim 24, where any one of R6-R15 is a photocrosslinkable or ionically crosslinkable group.

30. A polymer as in any one of claims 24-28, in which D is a straight or branched alkyl chain of 1-5 carbons, m is 0 or 1, and R2, R3, R4, R5, R5, and R7 are the same or different at each occurrence and are hydrogen, a straight or branched alkyl chain of 1-20 carbons, cycloalkyl, aryl, alkoxy, aryloxy, olefin, alkylamine, dialkylamine, arylamine, diarylamine, alkylamide, dialkylamide, arylamide, diarylamide, alkylaryl, or arylalkyl group.

31. A polymer of claim 24 comprised of repeating units of General Structure II, where L, N, and J are the same or different at each occurrence and are O, S, Se, N(H), or P(H).

32. A block or random copolymer as in claim 24 comprised of repeating units of general Structure III, where M, T, and Q are the same or different at each occurrence and are O, S, Se, N(H), or P(H), and R15 is a straight or branched alkyl chain of 1-5 carbons, unsubstituted or substituted with one or more hydroxyl, hydroxyether, carboxyl, carboxyester, carboxyamide, amino, mono- or di-substituted amino, thiol, thioester, sulfate, phosphate, phosphonate, or halogen substituents.

33. A higher order block or random copolymer comprised of three or more different repeating units, and having one or more repeating units as in any one of claims 24-32.

34. A block or random copolymer as in claim 24, which includes at least one terminal photopolymerizable group selected from the group consisting of amines, thiols, amides, phosphates, sulphates, hydroxides, alkenes, and alkynes.

35. A block or random copolymer as in claim 24 where X, Y, M is O, S, N—H, N—R, and wherein R is —H, CH₂, CR₂, Se or an isoelectronic species of oxygen.

36. A block or random copolymer as in claim 24 wherein an amino acid is attached to R₁, R₂, R₃, R₄, R₅, A, and/or Z.

37. A block or random copolymer as in claim 24 wherein a polypeptide is attached to R₁, R₂, R₃, R₄, R₅, A, and/or Z.

38. A block or random copolymer as in° claim 24 wherein an antibody is attached to R₁, R₂, R₃, R₄, R₅, A, and/or Z.

39. A block or random copolymer as in claim 24 wherein a nucleotide is attached to R₁, R₂, R₃, R₄, R₅, A, and/or Z.

40. A block or random copolymer as in claim 24 wherein a nucleoside is attached to R₁, R₂, R₃, R₄, R₅, A, and/or Z.

41. A block or random copolymer as in claim 24 wherein an oligonucleotide is attached to R₁, R₂, R₃, R₄, s, A, and/or Z.

42. A block or random copolymer as in claim 24 wherein a ligand is attached to R₁, R₂, R₃, R₄, R₅, A, and/or Z that binds to a biological receptor.

43. A block or random copolymer as in claim 24 wherein a pharmaceutical agent is attached to R₁, R₂, R₃, R₄, R₅, A, and/or Z.

44. A crosslinkable or noncrosslinkable polymer or copolymer according to claim 1 wherein the polymer is a dendritic macromolcule including at least one polymer selected from the group consisting of dendrimers, hybrid linear-dendrimers, or hyperbranched polymers according to one of the general formulas below: 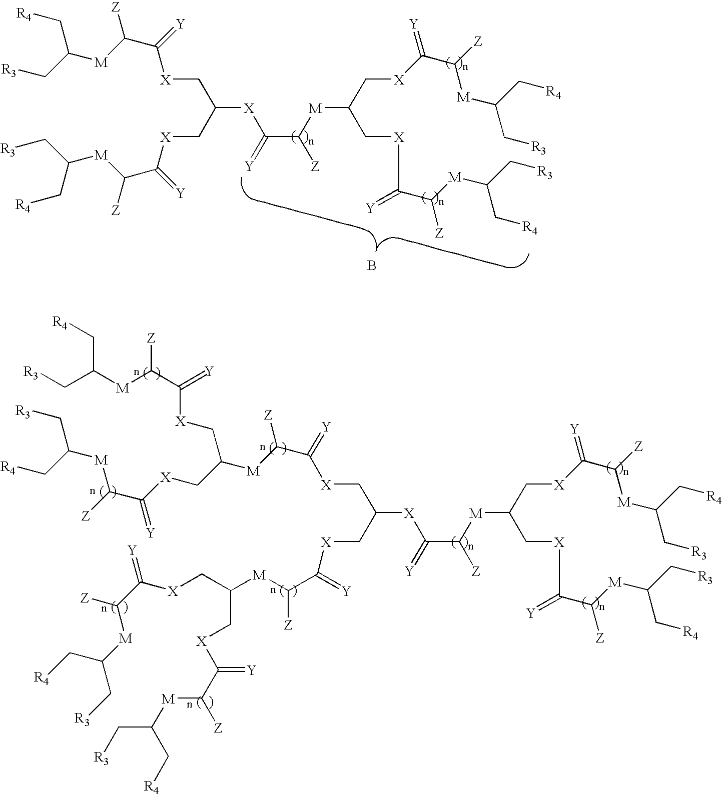 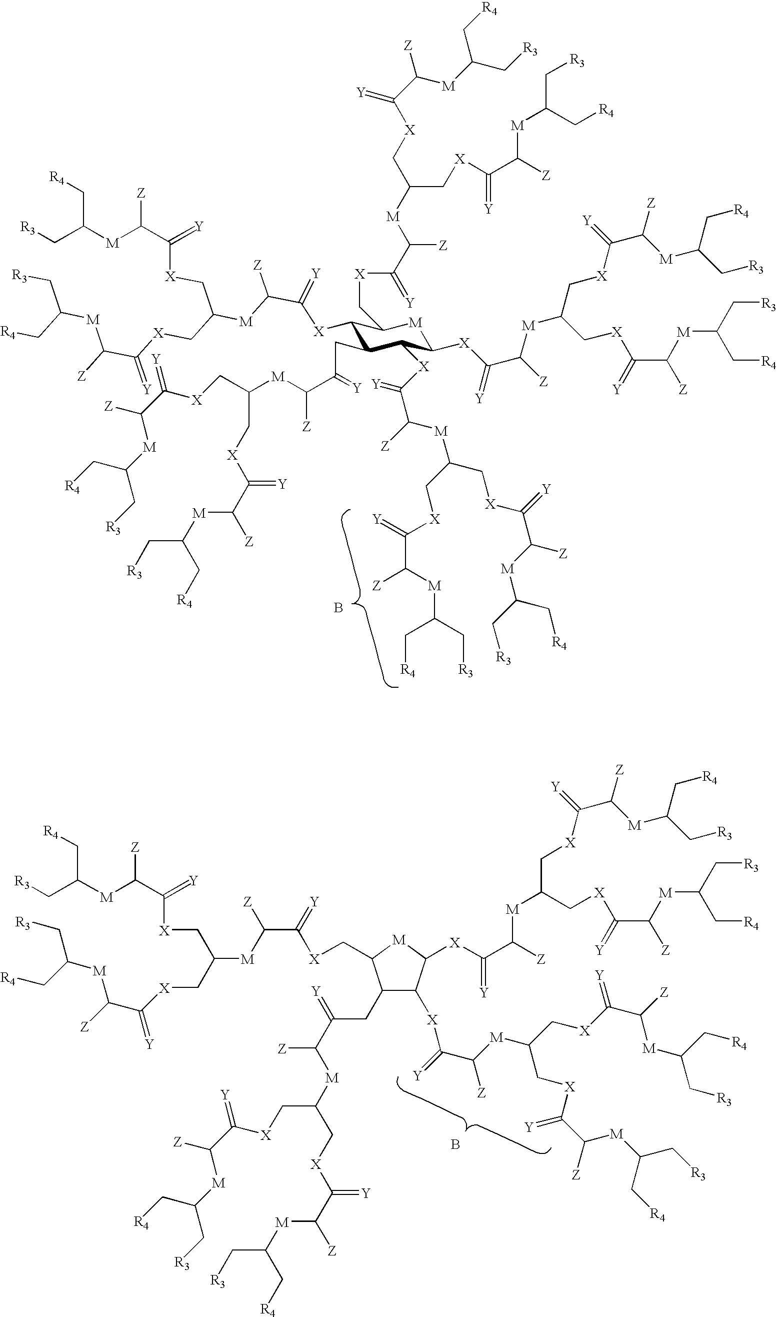 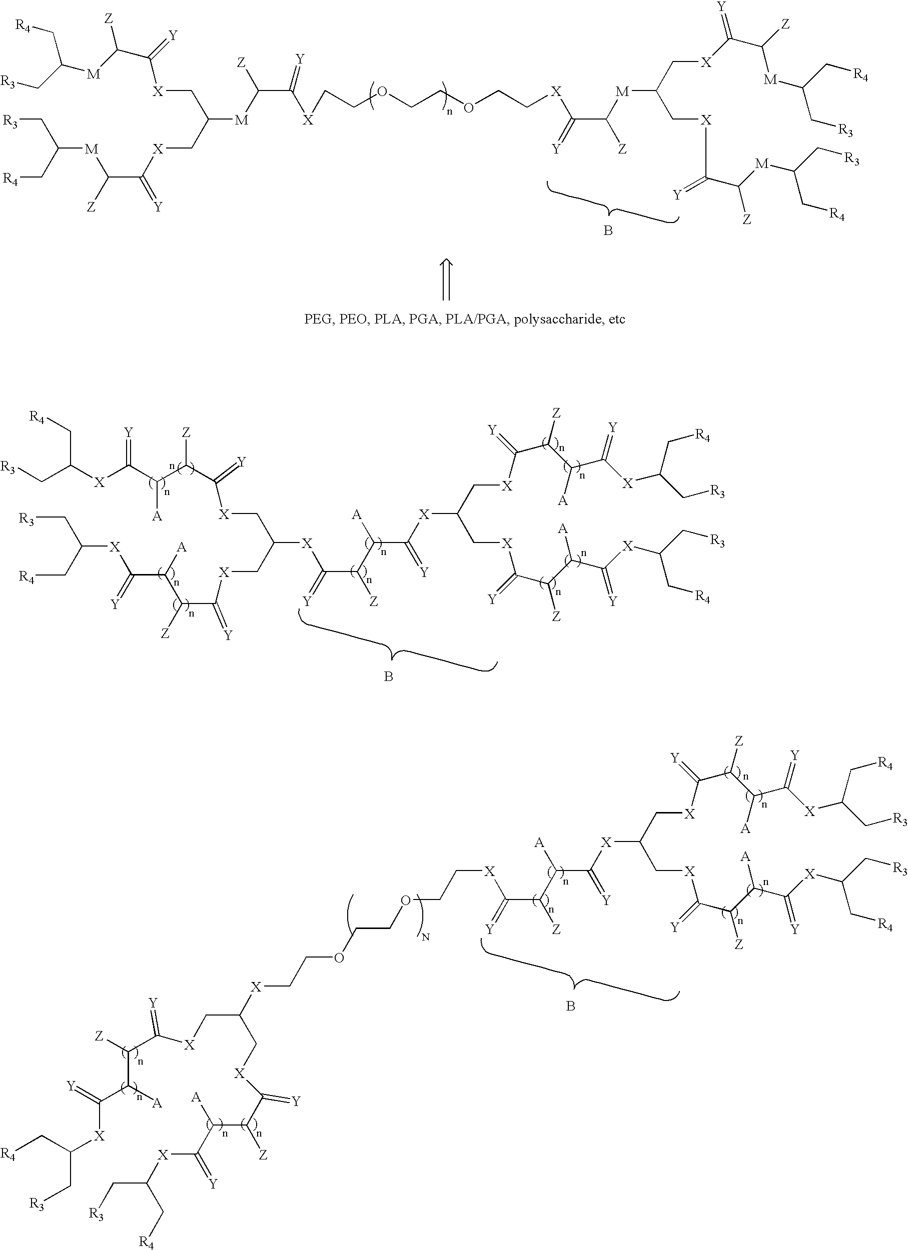 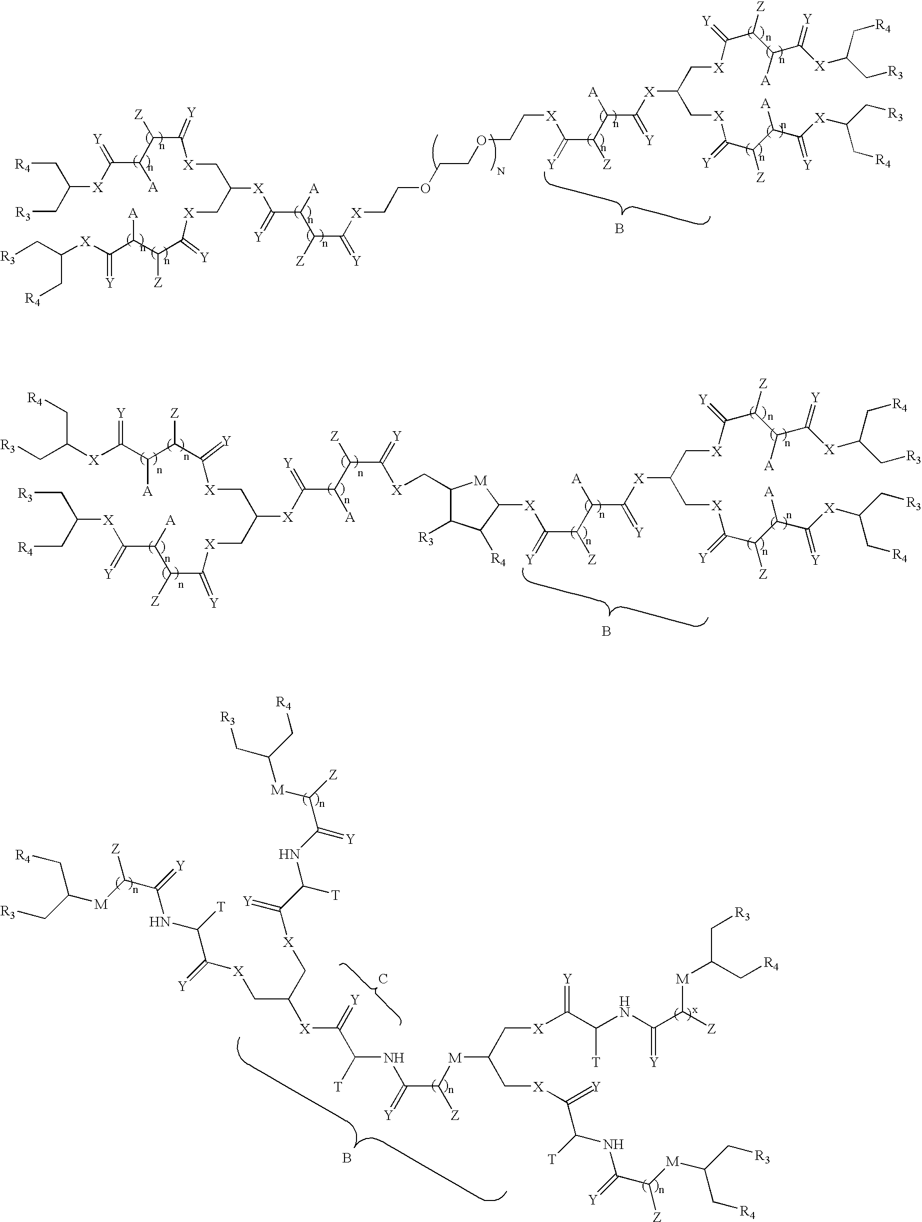 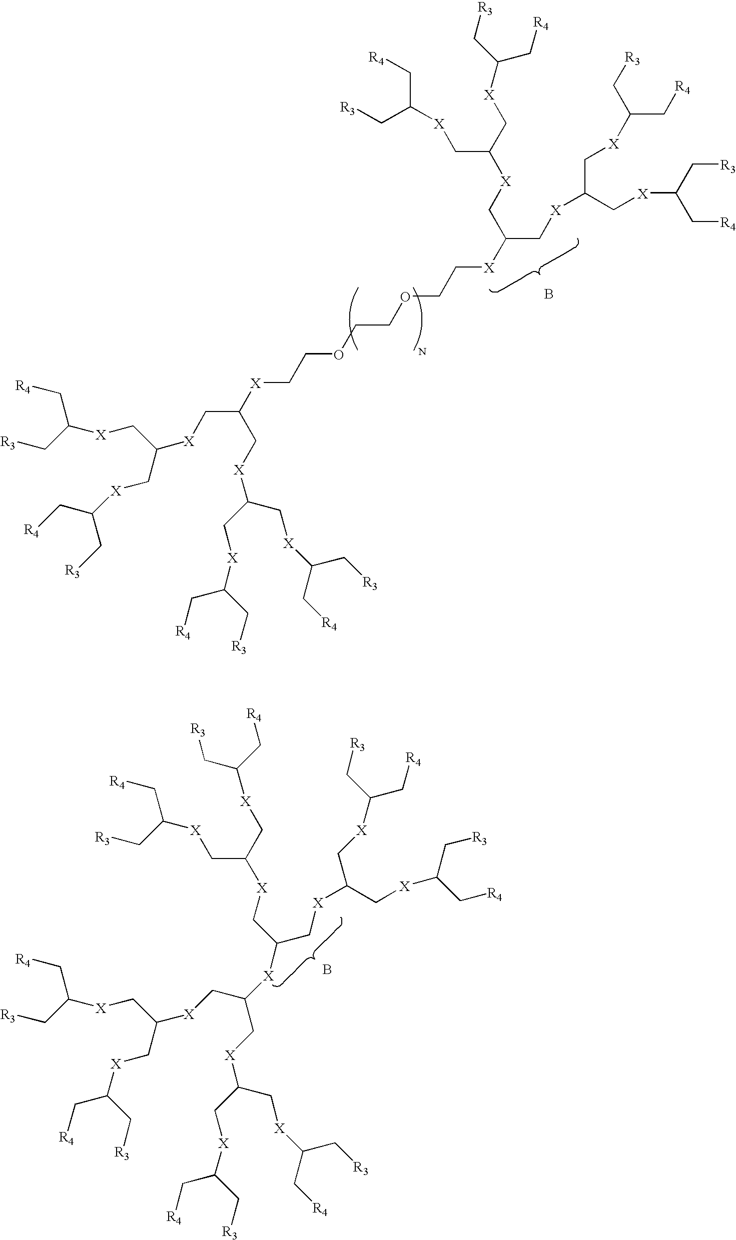 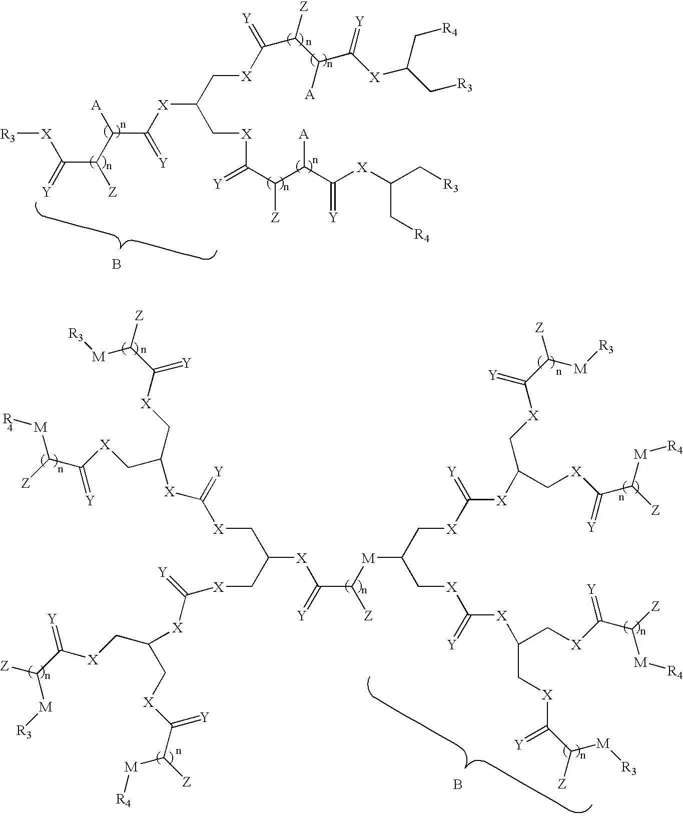
wherein R₁, R₂, R₅, A or Z, which may be the same or different, are —H, —CH₃, —OH, methoxy, carboxylic acids, sulfates, phosphates, aldehydes, amines, amides, thiols, disulfides, straight or branched chain alkanes, straight or branched chain alkenes, straight or branched chain esters, straight or branched chain ethers, straight or branched chain silanes, straight or branched chain urethanes, straight or branched chains, carbonates, straight or branched chain sulfates, straight or branched chain phosphates, straight or branched chain thiol urethanes, straight or branched chain amines, straight or branched chain thiol urea, straight or branched chain thiol ethers, straight or branched chain thiol esters, and
wherein R3 and R4, which may be the same or different, are the same as groups R1, R2, R5, A and Z as defined above, or are repeat patters of B; and
wherein X, Y, M is O, S, N—H, N—R, where R is —H, CH₂, CR₂ or a chain as defined above, Se or any isoelectronic species of oxygen; and n is 1-50.

45. The polymer of claim 44, where R₃ is a carboxycyclic acid protecting group such as but not limtied to a phthalimidomethyl ester, a t-butyldimethylsilyl ester, or a t-butyldiphenylsilyl ester.

46. The polymer of claim 44, where R₃, R₄, A, and Z are the same or different and are —H, —OH, —CH₃, carboxylic acid, sulfate, phosphate, aldehyde, methoxy, amine, amide, thiol, disulfide, straight or branched chain alkane, straight or branched chain alkene, straight or branched chain ester, straight or branched chain ether, straight or branched chain silane, straight or branched chain urethane, straight or branched chain, carbonate, straight or branched chain sulfate, straight or branched chain phosphate, straight or branched chain thiol urethane, straight or branched chain amine, straight or branched chain thiol urea, straight or branched chain thiol ether, straight or branched chain thiol ester, or a natural or un-natural amino acid.

47. The polymer of claim 44 which is fully saturated and/or fully unsaturated.

48. The polymer of claim 44 wherein straight or branched chains are the same number of carbons or different, and wherein R₁, R₂, R₃, R₄, R₅, A or Z are linked by at least one linker selected from the group consisting of esters, silanes, ureas, amides, amines, urethanes, thio]-urethanes, carbonates, thio-ethers, thio-esters, sulfates, phosphates and ethers.

49. The polymer of claim 44 wherein chains include at least one selected from hydrocarbons, flourocarbons, halocarbons, alkenes, and alkynes.

50. The polymer of claim 44 wherein said chains include polyethers, polyesters, polyamines, polyacrylic acids, polyamino acids, polynucleic acids and polysaccharides of molecular weight ranging from 200-1,000,000, and wherein said chain contains 1 or more photopolymerizable group.

51. The polymer of claim 44, wherein the chains include at least one of PEG, PLA, PGA, PGLA, and PMMA.

52. A block or random copolymer as in claim 51, which includes at least one terminal photopolymerizable group selected from the group consisting of amines, thiols, amides, phosphates, sulphates, hydroxides, alkenes, and alkynes.

53. The polymer of claim 44, wherein an amino acid is attached to Z, A, R₃, and/or R₄.

54. The polymer of claim 44, wherein a polypeptide is attached to Z, A, R₃, and/or R₄.

55. The polymer of claim 44, wherein an antibody is attached to Z, A, R₃, and/or R₄.

56. The polymer of claim 44, wherein a nucleotide is attached to Z, A, R₃, and/or R₄.

57. The polymer of claim 44, wherein a nucleoside is attached to Z, A, R₃, and/or R₄.

58. The polymer of claim 44, wherein an oligonucleotide is attached to Z, A, R₃, and/or R₄.

59. The polymer of claim 44, wherein a ligand is attached to Z, A, R₃, and/or R₄ that binds to a biological receptor.

60. The polymer of claim 44, wherein a pharmaceutical agent is attached to Z, A, R₃, and/or R₄.

61. The polymer of claim 44, wherein a carbohydrate is attached to Z, A, R₃, and/or R₄.

62. The polymer of claim 44, wherein a PET or MRI contrast agent is attached to Z, A, R₃, and/or R₄.

63. The polymer of claim 44, wherein the contrast agent is Gd(DPTA).

64. The polymer of claim 44, wherein an iodated compound for X-ray imagaging is attached to Z, A, R₃, and/or R₄.

65. The polymer of claim 44, wherein a pharmaceutical agent is attached to Z, A, R₃, and/or R₄ and is at least one selected from the group consisting of antibacterial, anticancer, anti-inflammatory, and antiviral.

66. The polymer of claim 44, wherein the carbohydrate is mannose or sialic acid.

67. A polymer of claim 44 which comprises a chain which is a polymer or copolymer of a polyester, polyamide, polyether, or polycarbonate of: 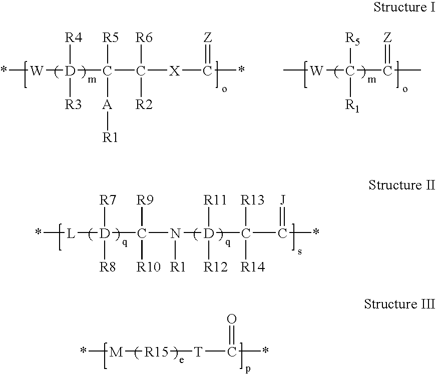
wherein R6-R15, which may be the same or different are —H, —CH₃, —OH, methoxy, carboxylic acids, sulfates, phosphates, aldehydes, amines, amides, thiols, disulfides, straight or branched chain alkanes, straight or branched chain alkenes, straight or branched chain esters, straight or branched chain ethers, straight or branched chain silanes, straight or branched chain urethanes, straight or branched chains, carbonates, straight or branched chain sulfates, straight or branched chain phosphates, straight or branched chain thiol urethanes, straight or branched chain amines, straight or branched chain thiol urea, straight or branched chain thiol ethers, straight or branched chain thiol esters, and
and wherein each of o, s and p is a number between 1 to 10000, and each of m, q, r and e is a number between 1 to 10.

68. A block or random copolymer as in claim 67, which includes at least one terminal photopolymerizable group selected from the group consisting of amines, thiols, amides, phosphates, sulphates, hydroxides, alkenes, and alkynes.

69. The polymer of claim 67, wherein an amino acid is attached to Z, A, R₃, and/or R₄.

70. The polymer of claim 67, wherein a polypeptide is attached to Z, A, R₃, and/or R₄.

71. The polymer of claim 67, wherein an antibody is attached to Z, A, R₃, and/or R₄.

72. The polymer of claim 67, wherein a nucleotide is attached to Z, A, R₃, and/or R₄.

73. The polymer of claim 67, wherein a nucleoside is attached to Z, A, R₃, and/or R₄.

74. The polymer of claim 67, wherein an oligonucleotide is attached to Z, A, R₃, and/or R₄.

75. The polymer of claim 67, wherein a ligand is attached to Z, A, R₃, and/or R₄ that binds to a biological receptor.

76. The polymer of claim 67, wherein a pharmaceutical agent is attached to Z, A, R₃, and/or R₄.

77. The polymer of claim 67, wherein a carbohydrate is attached to Z, A, R₃, and/or R₄.

78. The polymer of claim 67, wherein a PET or MRI contrast agent is attached to Z, A, R₃, and/or R₄.

79. The polymer of claim 67, wherein the contrast agent is Gd(DPTA).

80. The polymer of claim 67, wherein an iodated compound for X-ray imagaging is attached to Z, A, R₃, and/or R₄.

81. The polymer of claim 67, wherein a pharmaceutical agent is attached to Z, A, R₃, and/or R₄ and is at least one selected from the group consisting of antibacterial, anticancer, anti-inflammatory, and antiviral.

82. The polymer of claim 67, wherein the carbohydrate is mannose or sialic acid.

83. A surgical procedure which comprises using a photopolyerizable polymer or copolymer according to claim 1.

84. The surgical procedure as in claim 83, which is at least one selected from the group consisting of ophthalmic procedures, cardiovascular procedures, plastic surgery procedures, orthopedic procedures, gynecological procedures, ENT procedures, brain procedures, plastic surgery and skin procedures.

85. The surgical procedure of claim 83, wherein said photopolymerizable polymer or copolymer is dissolved or suspended in an an aqueous solution wherein the said aqueous solution is selected from water, buffered aqueous media, saline, buffered saline, solutions of amino acids, solutions of sugars, solutions of vitamins, solutions of carbohydrates or combinations of any two or more thereof.

86. The surgical procedure of claim 83 wherein the supramolecular structure of the dendrimer is a liposome or vesicle.

87. The surgical procedure of claim 83, wherein said photopolymerizable polymer or copolymer is dissolved or suspended in an non-aqueous liquid such as soybean oil, mineral oil, corn oil, rapeseed oil, coconut oil, olive oil, saflower oil, cottonseed oil, aliphatic, cycloaliphatic or aromatic hydrocarbons having 4-30 carbon atoms, aliphatic or aromatic alcohols having 1-30 carbon atoms, aliphatic or aromatic esters having 2-30 carbon atoms, alkyl, aryl or cyclic ethers having 2-30 carbon atoms, alkyl or aryl halides having 1-30 carbon atoms and optionally having more than one halogen substituent, ketones having 3-30 carbon atoms, polyalkylene glycol or combinations of any two or more thereof.

88. The surgical procedure of claim 83, wherein the supramolecular structure of the dendrimer is a micelle or emulsion.

89. The dendritic polymer or copolymer according to claim 1 which optionally contains at least one stereochemical center.

90. The dendritic polymer or copolymer of claim 89, wherein the at least one stereochemical center is chiral or achiral.

91. The dendritic polymer or copolymer according to claim 1 which optionally contains at least one site where branching is incomplete.

92. The dendritic polymer or copolymer according to claim 1 made by a convergent or divergent synthesis.
